(19) United States
(12) Patent Application Publication
(10) Pub. No.: US 2018/0112258 A1
(43) Pub. Date: Apr. 26, 2018

(54) MEDICAL USES AND METHODS FOR TREATING CANCER USING MONOPOLAR SPINDLE 1 (MPS1) KINASE INHIBITORS

(71) Applicants: THE INSTITUTE OF CANCER RESEARCH: ROYAL CANCER HOSPITAL, London Greater London (GB); BREAKTHROUGH BREAST CANCER, London Greater London (GB)

(72) Inventors: Mark Gurden, London Greater London (GB); Spyridon Linardopoulos, London Greater London (GB)

(21) Appl. No.: 15/566,015

(22) PCT Filed: Apr. 13, 2016
(86) PCT No.: PCT/EP2016/058121

§ 371 Date: Oct. 12, 2017

(30) Foreign Application Priority Data
Apr. 13, 2015 (GB) 1506248.2

(51) Int. Cl. C12Q 1/6827; A61K 31/4439; A61K 31/522; A61K 45/06; A61P 35/00; A61P 31/00

(52) U.S. Cl. CPC C12Q 1/6827; A61K 31/4439; A61P 31/00; A61K 45/06; A61P 35/00; A61K 31/522

(57) ABSTRACT

Medical uses and methods are provided for treating cancer using monopolar spindle 1 (MPS1) kinase inhibitors. Methods and uses for selecting MPS1 kinase inhibitors for use in treating cancer in a subject are provided, both in the initial selection of MPS1 kinase inhibitors and for addressing the development of acquired drug resistance that occur in the course of treatment.

### FIELD OF THE INVENTION

The present invention relates to medical uses and methods for treating cancer using monopolar spindle 1 (MPS1) kinase inhibitors, and in particular to methods and uses for selecting MPS1 kinase inhibitors for use in treating cancer in a subject, both in the initial selection of MPS1 kinase inhibitors and for addressing the development of acquired drug resistance that occur in the course of treatment.

### BACKGROUND OF THE INVENTION

In order for eukaryotic cells to undergo repetitive cell cycles, it is essential that a cell faithfully duplicates and then equally segregates their genome. The regulation of mitosis is achieved through an evolutionary conserved mechanism termed the spindle assembly checkpoint (SAC); an inhibitory signal that prevents metaphase to anaphase transition until all sister chromatid pairs are attached to mitotic spindle (via kinetochores; KT), in a bipolar orientation(1). MPS1 (monopolar spindle 1; also known as TTK) is a dual specificity serine, threonine and tyrosine kinase(2), which is vital for the recruitment of SAC proteins to unattached KTs, the formation of the mitotic checkpoint complex and therefore, the inhibition of the anaphase promoting complex/cyclosome (APC/C). Furthermore, MPS1 is also required for chromosome alignment and error-correction(3-5). Thus, following the inhibition of MPS1 kinase activity, cells prematurely exit mitosis with mis-attached/unaligned chromosomes, which causes severe chromosome mis-segregation, aneuploidy and cell death(6-10).

MPS1 has been suggested to be dysregulated in cancer cells; specifically, MPS1 mRNA expression is elevated in a number of cancers relative to normal tissue, including thyroid, breast, lung, bladder, and glioblastoma, higher levels correlating with a higher histological grade, aggressiveness and poor patient survival in breast cancer, glioblastoma and pancreatic ductal adenocarcinoma(11-17). Furthermore, PTEN-deficient breast cancer cell lines have been reported to be more sensitive to MPS1 depletion or kinase inhibition(18). As a result, MPS1 has attracted considerable attention as a potential drug target for anti-cancer therapy, with a number of small molecule inhibitors recently identified and under development(6-10, 19), or entering the clinic (BAY-1161909; clinical trial ID NCT02138812).

The selection of the optimum treatment for patients with cancer and the development of acquired resistance are some of the greatest challenges to the effectiveness of targeted therapies in the clinic. A number of different resistant mechanisms have been described, including: the up-regulation/switching to alternative signalling pathways, drug-efflux pumps and drug-resistant mutations. However, these discoveries have taken over 10 years; thus pre-emptively discovering inhibitors to target resistant mutations may have an important impact on overall patient survival. Accordingly, there is an unmet need in the art for approaches that help in the initial selection of MPS1 kinase inhibitors for treating patients with cancer and for addressing the selection of therapies that help address the development of acquired drug resistance that occur in the course of treatment.

### SUMMARY OF THE INVENTION

Broadly, the present invention is based on work carried out to elucidate the potential mechanisms that are capable of rendering cells resistant to MPS1 kinase inhibitors, examples of which are currently undergoing pre-clinical and clinical development. The present invention therefore addresses the problem of selecting MPS1 kinase inhibitors effective for the treatment of cancer in a subject, both in the initial selection of inhibitors and the selection of inhibitors that are capable of overcoming the effects acquired drug resistance that occur when monopolar spindle 1 (MPS1) kinase inhibitors are used to treat a tumour. The latter phenomenon may occur when most of an initial cancer cell population in a tumour contains a wild-type MPS1 kinase gene, so that treatment initially shrinks the tumour as most of the cell population within it is not resistant to the inhibitor. However, this can then leave a population of cells that are resistant to the inhibitor that can then begin to regrow. It would therefore be useful to know when a tumour has acquired resistance to a particular drug, and to understand which mutations are associated with the development of resistance to particular drugs. This in turn makes it possible to switch the drug being used in a therapy protocol to elicit a further response and to overcome the mutation causing the drug resistance.

With several MPS1 kinase inhibitors under pre-clinical development, the present invention aimed to investigate how cancer cells will develop resistance against these inhibitors. These initial experiments employed AZ3146 and NMS-P715, two of the first MPS1-specific inhibitors to be reported, and a recently identified inhibitor CCT251455. These experiments identified and characterized five point mutations in the kinase domain of MPS1 that render it resistant to a variety of MPS1 kinase inhibitors. Significantly, these mutations were pre-existing in all cancer cell lines and tumour samples tested, and even more strikingly, in lymphoblast samples from healthy individuals and normal breast tissues. Without wishing to be bound by any particular theory, the results suggest that these mutations are naturally occurring mutations, which are not introduced into the genome due to higher mutation rates in cancer cells and are only selected for upon inhibitor-treatment.

Structural studies showed that several MPS1 mutants conferred resistance by causing steric hindrance to inhibitor binding. Importantly, we show that these mutations occur in non-treated cancer cell lines and primary tumour samples and also pre-exist in normal lymphoblast and breast tissues. Furthermore, this finding was broadened to show that the most common mutation conferring resistance to gefitinib treatment, the EGFR p.T790M mutation, is also pre-existing in cancer cell lines and normal tissue. The data therefore suggest that mutations conferring resistance to targeted therapy are naturally occurring mutations in normal and cancer cells that are not introduced due to cancer cells being more mutagenic.

MPS1 (monopolar spindle 1; also known as TTK) is a dual specificity serine, threonine and tyrosine kinase(2), which is vital for the recruitment of SAC proteins to unattached KTs, the formation of the mitotic checkpoint complex and therefore, the inhibition of the anaphase promoting complex/cyclosome (APC/C). The HUGO Gene Symbol report for MPS1 can be found at http://www.ncbi.nlm.nih.gov/nuccore/XM_011536100.1 (GeneID:7272), which provides links to the MPS1 nucleic acid and amino acid sequences, as well as reference to the homologous murine and rat proteins. The amino acid sequence of human MPS1 is set out in SEQ ID NO: 1 and the nucleic acid sequence is set out in SEQ ID NO: 2.

The amino acid sequence of human MPS1 (SEQ ID NO:1) is as follows:

MESEDLSGRELTIDSIMNKVRDIKNKFKNEDLTDELSLNKISADTTDNSG
TVNQIMMMANNPEDWLSLLLKLEKNSVPLSDALLNKLIGRYSQAIEALPP
DKYGQNESFARIQVRFAELKAIQEPDDARDYFQMARANCKKFAFVHISFA
QFELSQGNVKKSKQLLQKAVERGAVPLEMLEIALRNLNLQKKQLLSEEEK
KNLSASTVLTAQESFSGSLGHLQNRNNSCDSRGQTTKARFLYGENMPPQD
AEIGYRNSLRQTNKTKQSCPFGRVPVNLLNSPDCDVKTDDSVVPCFMKRQ
TSRSECRDLVVPGSKPSGNDSCELRNLKSVQNSHFKEPLVSDEKSSELII
TDSITLKNKTESSLLAKLEETKEYQEPEVPESNQKQWQSKRKSECINQNP
AASSNHWQIPELARKVNTEKHTTFEQPVFSVSKQSPPISTSKWFDPKSIC
KTPSSNTLDDYMSCFRTPVVKNDFPPACQLSTPYGQPACFQQQQHQILAT
PLQNLQVLASSSANECISVKGRIYSILKQIGSGGSSKVFQVLNEKKQIYA
IKYVNLEEADNQTLDSYRNEIAYLNKLQQHSDKIIRLYDYEITDQYIYMV
MECGNIDLNSWLKKKKSIDPWERKSYWKNMLEAVETIHQHGIVHSDLKPA
NFLIVDGMLKLIDFGIANQMQPDTTSVVKDSQVGTVNYMPPEAIKDMSSS
RENGKSKSKISPKSDVWSLGCILYYMTYGKTPFQQIINQISKLHAIIDPN
HEIEFPDIPEKDLQDVLKCCLKRDPKQRISIPELLAHPYVQIQTHPVNQM
AKGTTEEMKYVLGQLVGLNSPNSILKAAKTLYEHYSGGESHNSSSSKTFE
KKRGKK

The nucleic acid sequence of human MPS1 (SEQ ID NO:2) is as follows:

GAAATGGAATCCGAGGATTTAAGTGGCAGAGAATTGACAATTGATTCCATAATGAACAAAGTGAGAGA
CATTAAAAATAAGTTTAAAAATGAAGACCTTACTGATGAACTAAGCTTGAATAAAATTTCTGCTGATA
CTACAGATAACTCGGGAACTGTTAACCAAATTATGATGATGGCAAACAACCCAGAGGACTGGTTGAGT
TTGTTGCTCAAACTAGAGAAAAACAGTGTTCCGCTAAGTGATGCTCTTTTAAATAAATTGATTGGTCG
TTACAGTCAAGCAATTGAAGCGCTTCCCCCAGATAAATATGGCCAAAATGAGAGTTTTGCTAGAATTC
AAGTGAGATTTGCTGAATTAAAAGCTATTCAAGAGCCAGATGATGCACGTGACTACTTTCAAATGGCC
AGAGCAAACTGCAAGAAATTTGCTTTTGTTCATATATCTTTTGCACAATTTGAACTGTCACAAGGTAA
TGTCAAAAAAAGTAAACAACTTCTTCAAAAAGCTGTAGAACGTGGAGCAGTACCACTAGAAATGCTGG
AAATTGCCCTGCGGAATTTAAACCTCCAAAAAAAGCAGCTGCTTTCAGAGGAGGAAAAGAAGAATTTA
TCAGCATCTACGGTATTAACTGCCCAAGAATCATTTTCCGGTTCACTTGGGCATTTACAGAATAGGAA
CAACAGTTGTGATTCCAGAGGACAGACTACTAAAGCCAGGTTTTTATATGGAGAGAACATGCCACCAC
AAGATGCAGAAATAGGTTACCGGAATTCATTGAGACAAACTAACAAAACTAAACAGTCATGCCCATTT
GGAAGAGTCCCAGTTAACCTTCTAAATAGCCCAGATTGTGATGTGAAGACAGATGATTCAGTTGTACC
TTGTTTTATGAAAAGACAAACCTCTAGATCAGAATGCCGAGATTTGGTTGTGCCTGGATCTAAACCAA
GTGGAAATGATTCCTGTGAATTAAGAAATTTAAAGTCTGTTCAAAATAGTCATTTCAAGGAACCTCTG
GTGTCAGATGAAAAGAGTTCTGAACTTATTATTACTGATTCAATAACCCTGAAGAATAAAACGGAATC
AAGTCTTCTAGCTAAATTAGAAGAAACTAAAGAGTATCAAGAACCAGAGGTTCCAGAGAGTAACCAGA
AACAGTGGCAATCTAAGAGAAAGTCAGAGTGTATTAACCAGAATCCTGCTGCATCTTCAAATCACTGG
CAGATTCCGGAGTTAGCCCGAAAAGTTAATACAGAGAAACATACCACTTTTGAGCAACCTGTCTTTTC
AGTTTCAAAACAGTCACCACCAATATCAACATCTAAATGGTTTGACCCAAAATCTATTTGTAAGACAC
CAAGCAGCAATACCTTGGATGATTACATGAGCTGTTTTAGAACTCCAGTTGTAAAGAATGACTTTCCA
CCTGCTTGTCAGTTGTCAACACCTTATGGCCAACCTGCCTGTTTCCAGCAGCAACAGCATCAAATACT
TGCCACTCCACTTCAAAATTTACAGGTTTTAGCATCTTCTTCAGCAAATGAATGCATTTCGGTTAAAG
GAAGAATTTATTCCATATTAAAGCAGATAGGAAGTGGAGGTTCAAGCAAGGTATTTCAGGTGTTAAAT
GAAAAGAAACAGATATATGCTATAAAATATGTGAACTTAGAAGAAGCAGATAACCAAACTCTTGATAG
TTACCGGAACGAAATAGCTTATTTGAATAAACTACAACAACACAGTGATAAGATCATCCGACTTTATG
ATTATGAAATCACGGACCAGTACATCTACATGGTAATGGAGTGTGGAAATATTGATCTTAATAGTTGG
CTTAAAAAGAAAAAATCCATTGATCCATGGGAACGCAAGAGTTACTGGAAAAATATGTTAGAGGCAGT
TCACACAATCCATCAACATGGCATTGTTCACAGTGATCTTAAACCAGCTAACTTTCTGATAGTTGATG
GAATGCTAAAGCTAATTGATTTTGGGATTGCAAACCAAATGCAACCAGATACAACAAGTGTTGTTAAA
GATTCTCAGGTTGGCACAGTTAATTATATGCCACCAGAAGCAATCAAAGATATGTCTTCCTCCAGAGA
GAATGGGAAATCTAAGTCAAAGATAAGCCCCAAAAGTGATGTTTGGTCCTTAGGATGTATTTTGTACT
ATATGACTTACGGGAAAACACCATTTCAGCAGATAATTAATCAGATTTCTAAATTACATGCCATAATT
GATCCTAATCATGAAATTGAATTTCCCGATATTCCAGAGAAAGATCTTCAAGATGTGTTAAAGTGTTG
TTTAAAAAGGGACCCAAAACAGAGGATATCCATTCCTGAGCTCCTGGCTCATCCATATGTTCAAATTC
AAACTCATCCAGTTAACCAAATGGCCAAGGGAACCACTGAAGAAATGAAATATGTTCTGGGCCAACTT
GTTGGTCTGAATTCTCCTAACTCCATTTTGAAAGCTGCTAAAACTTTATATGAACACTATAGTGGTGG
TGAAAGTCATAATTCTTCATCCTCCAAGACTTTTGAAAAA7AAAGGGGAAAAAAATGATTTGCAGTTA
TTCGTAATGTCAGATACCACCTATAAAATATATTGGACTGTTATACTCTTGAATCCCTGTGGAAATCT
ACATTTGAAGACAACATCACTCTGAAGTGTTATCACCAAAAAAAATTCAGTAGATTATCTTTAAAAGA
AAACTGTAAAAATAGCAACCACTTATGGCACTGTATATATTGTAGACTTGTTTTCTCTGTTTTATGCT
CTTGTGTAATCTACTTGACATCATTTTACTCTTGGAATAGTGGGTGGATAGCAAGTATATTCTAAAAA
ACTTTGTAAATAAAGTTTTGTGGCTAAAATGACACTAACATTT

According, in a first aspect the present invention provides a monopolar spindle 1 kinase (MPS1) inhibitor for use in a method of treating cancer in a human subject, wherein the method comprises:
(a) determining in a sample obtained from the subject whether a population of cells contains a MPS1 gene or a MPS1 protein that comprises one or more naturally occurring mutations as compared to the nucleic acid sequence of SEQ ID NO: 2 or the amino acid sequence of SEQ ID NO: 1, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y568C and p.C604W;
(b) selecting a MPS1 kinase inhibitor effective for use in treating the subject that is not associated with the development of acquired drug resistance that is correlated with the presence of the one or more naturally occurring mutations in the MPS1 gene or the MPS1 protein in step (a); and
(c) treating the subject with a therapy protocol that comprises administering the MPS1 kinase inhibitor selected in step (b).

In a further aspect, the present invention provides a method of treating a human cancer subject with a therapy protocol that comprises administration of a first monopolar spindle 1 kinase (MPS1) kinase inhibitor to the subject, the method comprising:
(a) determining in a sample obtained from the subject whether a population of cells contains a MPS1 gene or a MPS1 protein that comprises one or more naturally occurring mutations, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y568C and p.C604W;
(b) selecting a MPS1 kinase inhibitor effective for use in treating the subject that is not associated with the development of acquired drug resistance that is correlated with the presence of the one or more naturally occurring mutations in the MPS1 gene or the MPS1 protein in step (a); and
(c) treating the subject with the MPS1 kinase inhibitor selected in step (b).

In a further aspect, the present invention provides a method of treating a human cancer subject with a therapy protocol that comprises administration of a first monopolar spindle 1 kinase (MPS1) kinase inhibitor to the subject, the method comprising:
(a) determining in a sample obtained from the subject whether a population of cells contains a MPS1 gene or a MPS1 protein that comprises one or more naturally occurring mutations, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y568C and p.C604W;
   wherein the step of determining whether the MPS1 gene comprises one of more said mutations comprises amplifying by PCR and sequencing the MPS1 gene, or using SNP assays such as droplet digital PCR (ddPCR);
(b) selecting a MPS1 kinase inhibitor effective for use in treating the subject that is not associated with the development of acquired drug resistance that is correlated with the presence of the one or more naturally occurring mutations in the MPS1 gene or the MPS1 protein in step (a); and
(c) treating the subject with the MPS1 kinase inhibitor selected in step (b).

In a further aspect, the present invention provides a method of treating a human cancer subject with a therapy protocol that comprises administration of a first monopolar spindle 1 kinase (MPS1) kinase inhibitor to the subject, the method comprising:
(a) having determined in a sample obtained from the subject whether a population of cells contains a MPS1 gene or a MPS1 protein that comprises one or more naturally occurring mutations, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y568C and p.C604W;
   the step of having determined whether the MPS1 gene comprises one of more said mutations comprised amplifying by PCR and sequencing the MPS1 gene, or using SNP assays such as droplet digital PCR (ddPCR);
(b) selecting a MPS1 kinase inhibitor effective for use in treating the subject that is not associated with the development of acquired drug resistance that is correlated with the presence of the one or more naturally occurring mutations in the MPS1 gene or the MPS1 protein in step (a); and
(c) treating the subject with the MPS1 kinase inhibitor selected in step (b).

In a further aspect, the present invention provides a method of selecting a monopolar spindle 1 kinase (MPS1) kinase inhibitor for use in treating cancer in a human subject, the method comprising:
(a) determining in a sample obtained from the subject whether a population of cells contains a MPS1 gene or a MPS1 protein that comprises one or more naturally occurring mutations, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y568C and p.C604W;
(b) selecting a MPS1 kinase inhibitor effective for use in treating the subject that is not associated with the development of acquired drug resistance that is correlated with the presence of the one or more naturally occurring mutations in the MPS1 gene or the MPS1 protein in step (a); and
(c) treating the subject with a therapy protocol that comprises administering the MPS1 kinase inhibitor selected in step (b).

In some embodiments, the medical uses and method of the present invention are employed for the selection of MPS1 kinase inhibitor which is likely to be effective for the treatment of a subject initially diagnosed with a cancer treatable using MPS1 kinase inhibitors, for example to avoid treatment with an inhibitor to which the cancer is resistant. Alternatively or additionally, the present invention can be used in the course of ongoing treatment of a subject with cancer, for example monitoring the subject during treatment with the MPS1 inhibitor to determine whether cancer cells from the subject have developed acquired drug resistance; and optionally selecting a further MPS1 kinase inhibitor for use in treating the subject, or alternative treatment.

In a further aspect, the present invention provides a method of determining a therapy protocol using a monopolar spindle 1 kinase (MPS1) kinase inhibitor for treating cancer in a human subject, the method comprising:
(a) determining whether the subject has acquired resistance to treatment with a first MPS1 kinase inhibitor;
(b) determining in a sample obtained from the subject whether a population of cells contains a MPS1 gene or a MPS1 protein that comprises one or more naturally occurring mutations, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y5680 and p.C604W and wherein the presence of one or more mutations is indicative of a resistance to the first MPS1 inhibitor;
(c) selecting a further MPS1 kinase inhibitor effective for use in treating the subject that is not associated with the development of acquired drug resistance that is correlated with the presence of the one or more naturally occurring mutations in the MPS1 gene or the MPS1 protein in step (b); and
(d) treating the subject with a revised therapy protocol that comprises administering the further MPS1 kinase inhibitor selected in step (c).

Embodiments of the present invention will now be described by way of example and not limitation with reference to the accompanying figures. However various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### BRIEF DESCRIPTION OF THE FIGURES

FIG. 1: Generation of HCT116 cell lines resistance to AZ3146 and identification of p.S611G and p.I531M mutations in MPS1

(A) The structure of AZ3146. Line graph of cell viability assays of parental (solid line), AzR1 (short and long dashed line) and AzR3 (dashed line) HCT116 cells to AZ3146.

(B) 14-day clonogenic assays showing the viability of HCT116 cell lines to AZ3146.

(C) Sequencing chromatograms of AZ3146-resistant clones AzR1 and AzR3, compared to the parental cell line. Stars indicate the mutated base.

(D) Flow cytometry cell cycle profiles of HCT116 cells (parental, AzR1 and AzR3) treated for 24 hours with AZ3146.

(E) Box-and-whisper plot showing the time HCT116 cells (transfected with Histone H2B-mCherry) spent in mitosis, in the absence and presence of AZ3146. The boxes represent the interquartile ranges and the whisker the full range. The result was analysed by One-way ANOVA with *** indicating p<0.0001 and ns indicating not significant. N=>45 cells per condition.

(F) The structures of ONCOII, SNG12 and NMS-P715. Line graph of cell viability assays of parental (solid line), AzR1 (short and long dashed line) and AzR3 (dashed line) HCT116 cells to the indicated compounds.

(G) Line graph of cell viability assays of tet-inducible DLD1 cells expressing wild-type (WT+tet; thick short dashed line), p.I531M (long thin dashed line), p.S611G (dotted line) and Db1 (short and long dashed line) MPS1 constructs, compared to un-induced wild-type control (WT−tet; solid line).

(H) IP-kinase assays of the indicated Myc-MPS1 constructs transfected into HEK293T cells. The relative activity (RA) compared to wild-type (WT) construct is shown, as calculated by phosphorimager. Similar amounts of proteins were loaded as shown by SimplyBlue staining.

(I) Line graph showing the inhibition of MPS1 T33/S37 auto-phosphorylation for Myc-tagged WT (solid line), p.S611G (short and long dashed line), p.I531M (dotted line) and Db1 (long thin dashed line) constructs in the presence of AZ3146.

All graphs represent the mean of three experiments +/−SD.

FIG. 2: The generation of HCT116 cell lines resistance to NMS-P715 and the identification of p.M600T, p.Y568C and P.C604W mutations in MPS1

(A) Sequencing chromatograms of NMS-P715-resistant clones NvR1, NvR11 and NvR12. Stars indicate the mutated base.

(B) Line graph of cell viability assays of HCT116 clones NvR1 (long thin dashed line), NvR11 (dotted line) and NvR12 (short and long dashed line) to NMS-P715-induced cell death, compared to the parental (solid line) cell line.

(C) A 14-day clonogenic assays showing the viability of HCT116 clones to NMS-P715.

(D) Flow cytometry cell cycle profiles of HCT116 cells (parental, NvR1, NvR11 and NvR12) treated for 24 hours with NMS-P715.

(E) Box-and-whisper plot showing the time HCT116 cells (transfected with Histone H2B-mCherry) spent in mitosis, in the absence and presence of NMS-P715. The boxes represent the interquartile ranges and the whisker the full range. The result was analysed by One-way ANOVA, with *** indicating p<0.0001 and ns indicating not significant. N=>40 cells per condition.

(F) Line graph of cell viability assays of tet-inducible DLD1 cells expressing M600T (solid lines), Y568C (long thin dashed line and short thick dashed line), and 0604W (dotted line and short and long dashed line) GFP-MPS1 constructs, in the absence (circles) and presence (squares) of tetracycline (tet).

(G) IP-kinase assays of the indicated Myc-MPS1 constructs transfected into HEK293T cells. The relative activity (RA) compared to wild-type (WT) MPS1 is shown, as calculated by phosphorimager. Similar amount of proteins were loaded as shown by SimplyBlue staining.

(H) Line graph showing the inhibition of MPS1 T33/S37 auto-phosphorylation for Myc-tagged WT (fine two dots and dash fine line), p.S611G (fine solid line), p.I531M (thick solid line), Db1 (short thick dashed line), p.M600T (long thin dashed line), p.Y568C (dotted line) and p.C604W (thick short and long dashed line) constructs in the presence of NMS-P715.

(I) Line graph of cell viability assays of parental (solid line), NvR1 (dashed line), NvR11 (dotted line) and NvR12 (short and long dashed line) HCT116 cells to the indicated MPS1 inhibitor, in cell viability assay.

All graphs represent the mean of three experiments +/−SD.

FIG. 3: CCT251455 is a specific and potent MPS1 inhibitor.

(A) Line graph of cell viability assays of HCT116 cells to CCT251455 in a 4-day cell viability assay. The structure of CCT251455 is shown.

(B) Line graph showing the inhibition of MPS1 T33/S37 (solid line) and T676 (dotted line) auto-phosphorylation for Myc-MPS1.

(C) Flow cytometry cell cycle profiles of HCT116 cells treated for 24, 48 and 72 hours with CCT251455.

(D) Top: Box-and-whisker plot showing the time HeLa cells (expressing Histone H2B-mCherry) spent in mitosis, in the absence and presence of 0.6 μM CCT251455. The boxes represent the interquartile ranges and the whisker the full range. The result was analysed by Student T test, being highly significantly different (p<0.0001). N=>72 cells per condition. Bottom: Bar graph quantifying mitotic defects. N: normal, Tri: tripolar, Lag: lagging chromosome, DC: division with unaligned chromosomes, ND: no anaphase division.

(E) Line graph of mitotic index, as judged by MPM2 staining and flow cytometry. Noc: nocodazole (squares), Tax: taxol (circles), w/out: washout drug (hatched fill), 455: treatment with 0.6 μM CCT251455 (white fill).

(F) Immunofluorescence images showing the localisation of the indicated kinetochore proteins in HeLa cells, in the absence or presence of 0.6 μM CCT251455. The white boxes are enlarged to highlight kinetochores.

(G) Line graph of cell viability assays of parental (thick short and long dashed line) and the indicated drug resistant HCT116 cell lines to CCT251455.

(H) Line graph showing the inhibition of MPS1 T33/S37 auto-phosphorylation for Myc-tagged WT (thick short and long dashed line) and the indicated mutant MPS1 constructs in the presence of CCT251455.

All graphs represent the mean of three experiments +/−SD.

FIG. 4: The p.S611G mutation has minor affects on the structure of MPS1-KD

(A) Comparison of WT (orange, paler shade) and p.S611G (blue, darker shade) MPS1 with AZ3146.

(B) Comparison of WT (orange, paler shade) and p.S611G (blue, darker shade) MPS1 with ONCOII. Activation loop and P-loop residues have been omitted for clarity.

(C) Structure of compound 1 and the comparison of it bound to WT (orange, paler shade) MPS1 (PDB code 4C4H, shown in green) and p.S611G (blue, darker shade) MPS1.

FIG. 5: The pI531M and p.C604W mutations prevent normal inhibitor binding to MPS1

(A) MPS1 WT structure with ATP (3HMN) showing three modeled rotamers of Met531. The grey surface represents the conformational space available to this residue in the absence of main chain movements. The three Met side chains are the most common rotamers of Met, which would not clash with the ribose group of ATP or the residues surrounding the Met531 side chain (Lys529 and Gln541).

(B) MPS1 WT structure with AZ3146 showing the position of 1531. All of the most common rotamers of Met531 are predicted to clash with the anilino or cyclopentyl groups of AZ3146, or with surrounding protein residues (Gln541, Lys529 or Cys604).

(C) Comparison of WT MPS1 (orange, paler shade) and p.C604W mutant MPS1 (purple, darker shade) with NMS-P715.

FIG. 6: Compound 2 and 3 inhibit the MPS1 p.C604W mutant

(A) Structures of compound 2 and 3.

(B) Line graph of cell viability assays of parental (dots and dashes line) and drug resistant HCT116 cell lines to compound 2 and 3 in a 4-day cell viability assay (the graph represents the mean of three experiments +/−SD).

(C) Flow cytometry profiles of parental, AzR1 and NvR12 HCT116 cells treated for 24 hours with compound 2.

(D) X-ray of WT MPS1 with compound 2. The electron density from an Fo-Fc omit map is shown as a mesh, contoured at 3.0sigma.

(E) Comparison of WT (orange; paler shade) and p.C604W (purple, darker shade) MPS1 with compound 2.

FIG. 7: MPS1 and EGFR drug-resistant mutations are pre-existing in cancer and normal cells

(A) ddPCR dot plots of mutations in parental and drug-resistant

HCT116 cells lines. Each quadrant represents droplets that contain: empty droplets (bottom left), the wild-type base only (bottom right), the mutant base only (top left), or both wild-type and mutant alleles (top right).

(B-C) Bar graphs showing the fractional abundance (FA) of each indicated mutant for (B) the mutant-containing cell lines and (C) the parental HCT116 cell line (the graph represents the mean of three experiments +/−SD).

(D) ddPCR dot plots of p.S611C, p.S611R and p.Y568Stop mutations in HCT116 cells, in the presence or absence of 100 ng DNA of the indicated mutant vectors.

(E-F) Fractional abundances of each mutation in breast tumour samples (e) and lymphoblast samples (F). Values equal to, or below the false positive rates are reported as 0.

(G) ddPCR dot plots for the EGFR p.T790M mutation in HCT116 cells alone (left) or with 100 fg ultramer spike (right).

FIG. 8: Expression of the p.S611G, p.I531M and Db1 MPS1 mutant constructs in DLD1 Flp-In TRex cells recues the spindle assembly checkpoint defect following AZ3146 treatment

(A) Immunoblot showing the induction of GFP-MPS1 constructs with tetracycline (tet) in DLD1 Flp-In TRex cells.

(B) Immunofluorescence images showing kinetochore localisation of GFP-MPS1 constructs. Boxes are enlarged to highlight kinetochores.

(C) Box-and-whisper plot showing the time DLD1 cells spent in mitosis, in the absence and presence of tetracycline (tet) and 2 μM AZD3146. The boxes represent the interquartile ranges and the whisker the full range. *** Signifies highly significantly different (p<0.0001) by one way ANOVA. NS: not significant. N=>118 cells per condition.

(D) Flow cytometry cell cycle profiles of DLD1 Flp-In TRex cells expressing MPS1 mutant constructs in the absence and presence of AZ3146 for 24 hours.

(E) Immunoblot showing override of a nocodazole-induced spindle assembly checkpoint, following AZ3146 treatment for 2 hours, in the absence and presence of tetracycline.

(F) Line graph of cell viability assay of DLD1 Flp-In TRex cells to NMS-P715 following expression of p.I531M, p.S611G and Db1 MPS1 constructs. The graph represents the mean of three experiments +/−SD.

(G) Immunoblot showing the inhibition of auto-phosphorylation of Myc-MPS1 constructs at T33/S37 and T676 following treatment with AZ3146.

(H) Immunoblot of HCT116 cells co-transfected with wild-type and p.S611G MPS1 constructs, showing the inhibition of MYC-MPS1 auto-phosphorylation, but not GFP-MPS1 p.S611G, at T33/S37 following AZ3146 treatment.

FIG. 9: Expression of the p.M600T, p.Y568C and p.C604W MPS1 mutant constructs in DLD1 Flp-In TRex cells recues the spindle assembly checkpoint defect following AZ3146 treatment

(A) Immunoblot showing the induction of GFP-MPS1 constructs with tetracycline (tet) in DLD1 Flp-In TRex cells. Boxes are enlarged to highlight kinetochores.

(B) Immunofluorescence images showing kinetochore localisation of GFP-MPS1 constructs.

(C) Flow cytometry cell cycle profiles of DLD1 Flp-In TRex cells expressing MPS1 mutant constructs in the absence and presence of NMS-P715 for 24 hours.

(D) Box-and-whisper plot showing the time DLD1 cells spent in mitosis, in the absence and presence of tetracycline (tet) and 1 μM NMS-P715. The boxes represent the interquartile ranges and the whisker the full range. *** Signifies highly significantly different (p<0.0001) by one way ANOVA. N=>105 cells per condition.

(E) Immunoblot showing override of a nocodazole-induced spindle assembly checkpoint, following NMS-P715 treatment for 2 hours.

(F) Immunoblot comparing the auto-phosphorylation of MYC-MPS1 constructs immunoprecipitated from nocodozole-arrested HCT116 cells.

FIG. 10: CCT251455 kills cancer cells by inhibiting the kinetochore recruitment of SAC protein

Immunofluorescence of HeLa cells to show the kinetochore localisation of proteins in the absence and presence of CCT251445. Cells were pre-treated for 1 hour with CCT251455 prior to being arrested in mitosis using nocodazole and MG132. The white boxes are enlarged to highlight the kinetochores.

FIG. 11: CCT251455-resistant HCT116 clones

Line graph of cell viability assay of HCT116 clones made resistant to CCT251455. The CCT251455-resistant clones were created being grown for 10 days in 0.16 μM CCT251455, then passaged and grown for a further 3 weeks in 0.5 μM CCT251455. The graph represents the mean of three experiments +/−SD.

FIG. 12: Crystal structures of AZD3146 and ONCOII bound to MPS1-KD

(A) WT MPS1 with AZ3146 shown in orange. The electron density from an Fo-Fc omit map is shown as a mesh, contoured at 3.0sigma.

(B) WT MPS1 with ONCOII shown in orange. The electron density from an Fo-Fc omit map is shown as a mesh, contoured at 3.0sigma.

FIG. 13: ddPCR analysis of drug-resistance mutations shows they are pre-existing in cancer cell lines and quickly introduced into a population of HCT116 cells

(A) Bar graphs to show the fractional abundance of the p.S611G mutation in HCT116 cells with increasing concentrations of gDNA.

(B) Bar graphs to show the fractional abundance of the p.S611G mutant in 100 ng of parental HCT116 gDNA spiked with 0.1-100 ng gDNA from the p.S611G-containing AzR1 cell line.

(C) A bar graph to show the fold increase in fractional abundance of MPS1 mutants in HCT116 cells grown in the presence of 0.8 μM AZ3146 for 5 days.

(D) A bar graph to show the fractional abundance of the MPS1 mutations in HCT116 clones expanded from single cells for 24 days.

(E) Bar graphs to show the fractional abundance of p.S611G (left) and other mutations (right) in AZR1 clones grown for 24 days from single cells.

(F) A bar graph to show the fractional abundance of the MPS1 p.S611G and EGFR p.T790M mutations in 5 normal breast tissue samples.

FIG. 14: Treatment of CAL51 cells with AZ3146 and NMS-P715 selected for the same p.S611G and p.Y568C MPS1 mutations

(A) Line graphs of cell viability assays of parental (dotted line) and p.S611G containing AZ3146-resistant HCT116 cell lines (solid line), treated with AZ3146 (left) and NMS-P715 (right) in a 4-day cell viability assay. The graph represents the mean of three experiments +/−SD.

(B) Line graphs of cell viability assays of parental (dotted line) and p.Y568C containing NMS-P715-resistant HCT116 cell lines (solid line), treated with NMS-P715 (left) and CCT251455 (right) in a 4-day cell viability assay. The graph represents the mean of three experiments +/−SD.

### DETAILED DESCRIPTION

### MPS1 Kinase Inhibitors

As the experiments described herein demonstrate mutations in the MPS1 gene or protein sequences are able to confer resistance against a number of structurally different MPS1 kinase inhibitors, and in particular to MPS1 kinase inhibitors that bind to the hinge region of MPS1 kinase domain. This in turn means that the medical uses and methods described herein are applicable to the general class of MPS1 kinase inhibitors, in addition to the specific compounds used in the examples. Accordingly, further MPS1 kinase inhibitors may be tested in analogous experiments to those described herein to determine whether their use leads to the development of acquired drug resistance characterised by the presence of one or more of the mutations found in the work described in the examples. The medical uses and methods of the present invention then allow the selection of a MPS1 kinase inhibitor for which the cancer cells of the tumour are not resistant.

Examples of MPS1 kinase inhibitors known in the art include:
(a) 8-oxapurines and their derivatives, such as AZ3146.
(b) Pyridine and pyrimidine derivatives, and more specifically diaminopyridines such as ONCOII, that are disclosed in WO 2011/016472.
(c) Triaminopyridine and their derivatives such Shionogi Compound 12.
(d) Pyrazolo-quinazolines, such as NMS-P715.
(e) MPS1 kinase inhibitors disclosed in WO 2012/123745 such as:
Tert-Butyl 6-(2-Chloro-4-(1-methyl-1H-imidazol-5-yl) phenylamino)-2-(1-methyl-1H-pyrazol-4-yl)-1H-pyrrolo[3,2-c]pyridine-1-carboxylate (CCT251455)
Isopropyl 6-(4-(1,2-dimethyl-1H-imidazol-5-yl)-2-phenylamino)-2-(1-methyl-1H-pyrazol-4-yl)-1H-pyrrolo[3,2-c]pyridine-1-carboxylate (Compound 2)
Isopropyl 6-(4-(1,2-dimethyl-1H-imidazol-5-yl)-2-fluorophenylamino)-2-(1-methyl-1H-pyrazol-4-yl)-1H-pyrrolo[3,2-c]pyridine-1-carboxylate (Compound 3)

All of these documents are hereby incorporated by reference or cross referenced with respect to the MPS1 kinase inhibitor compounds they disclose.

Examples of MPS1 kinase inhibitors in the clinic, in clinical trials or in pre-clinical development are set out in the Table below:

### Mps1 Inhibitors

| Name | Class | Structure | Reference |
|---|---|---|---|
| AZ3156 (Astrazeneca) | 8-oxopurine | 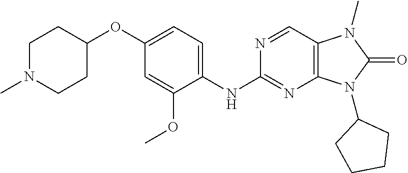 [SMILES: COc1cc(OC2CCN(C)CC2)ccc1Nc1ncc2c(n1)n(C1CCCC1)c(=O)n2C] | (7) |
| NMS-P715 | Pyrazolo- quinazoline | 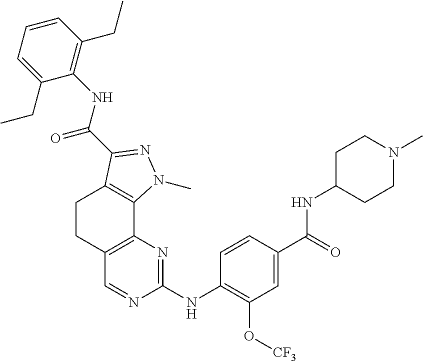 [SMILES: CCc1cccc(CC)c1NC(=O)c1nn(C)c2c1CCc1cnc(Nc3ccc(C(=O)NC4CCN(C)CC4)cc3OC(F)(F)F)nc1-2] | (6) |
| ONCOII (OncoTherapy Science Inc.) | Diaminopyridine | 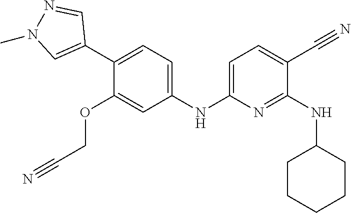 [SMILES: Cn1cc(-c2ccc(Nc3ccc(C#N)c(NC4CCCCC4)n3)cc2OCC#N)cn1] | (50) |
| SNG12 (Shionogi) | Triaminopyridine | 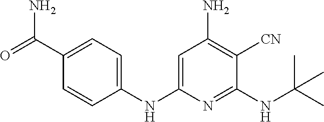 [SMILES: CC(C)(C)Nc1nc(Nc2ccc(C(N)=O)cc2)cc(N)c1C#N] | (33) |
| Mps-BAY1 (Bayer) | Triazolopyridine | 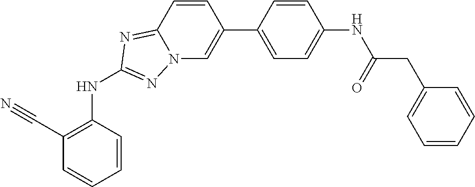 [SMILES: N#Cc1ccccc1Nc1nc2ccc(-c3ccc(NC(=O)Cc4ccccc4)cc3)cn2n1] | (8) |
| Mps-BAY2a/b (Bayer) | Imidazopyrazine | 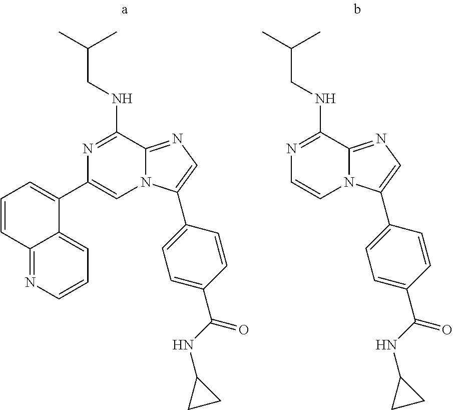 [SMILES: CC(C)CNc1nc(-c2cccc3ncccc23)cn2c(-c3ccc(C(=O)NC4CC4)cc3)cnc12 ; CC(C)CNc1nccn2c(-c3ccc(C(=O)NC4CC4)cc3)cnc12] | (8) |
| SP600125 | Anthrone derivative | 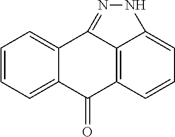 [SMILES: O=C1c2ccccc2-c2n[nH]c3cccc1c23] | (51) |
| Reversine | Substituted purine analogue | 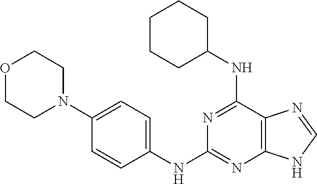 [SMILES: c1nc2c(NC3CCCCC3)nc(Nc3ccc(N4CCOCC4)cc3)nc2[nH]1] | (52) |
| MPI-0479605 (Myrexis, Inc.) | Substituted purine analogue | 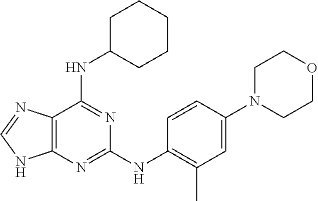 [SMILES: Cc1cc(N2CCOCC2)ccc1Nc1nc(NC2CCCCC2)c2nc[nH]c2n1] | (10) |
| Compound 32 | Indazole-based inhibitor | 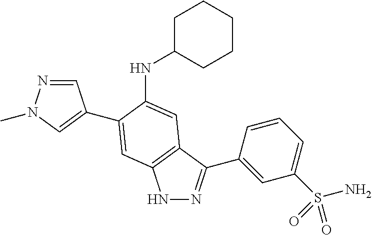 [SMILES: Cn1cc(-c2cc3[nH]nc(-c4cccc(S(N)(=O)=O)c4)c3cc2NC2CCCCC2)cn1] | (53) |
| CCT251455 | Pyrrolopyridine | 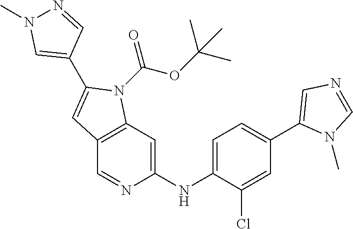 [SMILES: Cn1cc(-c2cc3cnc(Nc4ccc(-c5cncn5C)cc4Cl)cc3n2C(=O)OC(C)(C)C)cn1] | (19) |
| Compound 75 | 3-(4- (heterocyclyl) phenyl)-1H-indazole- 5-carboxamide | 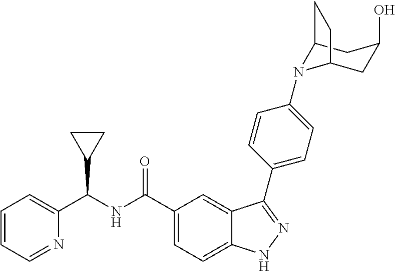 [SMILES: O=C(N[C@@H](c1ccccn1)C1CC1)c1ccc2[nH]nc(-c3ccc(N4C5CCC4CC(O)C5)cc3)c2c1] | (55) |
| Mps1-IN-2 | Aminopyrimidine | 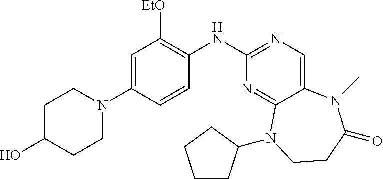 [SMILES: CCOc1cc(N2CCC(O)CC2)ccc1Nc1ncc2c(n1)N(C1CCCC1)CCC(=O)N2C] | (9) |
| GNE-7915 | Aminopyrimidine | 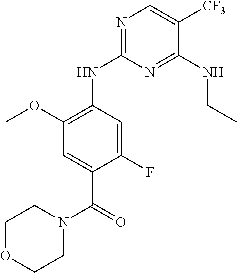 [SMILES: CCNc1nc(Nc2cc(F)c(C(=O)N3CCOCC3)cc2OC)ncc1C(F)(F)F] | (56) |

The present invention identifies and characterises five point mutations in the kinase domain of MPS1 that confer resistance against multiple inhibitors. The mutations are: p.I531M, p.S611G, p.M600T, p.Y568C and p.C604W and the inhibitors tested were AZ3146, ONCO II, SNG12, NMS-P715, CCT251455, Compound 2 and Compound 3. It was found that different inhibitors are effective against distinct mutations, as summarized in the following table:

| | | Mutations | | | | |
|---|---|---|---|---|---|---|
| | | p.I531M | p.S611G | p.M600T | p.Y568C | p.C604W |
| Inhibitors | AZ3146 | R | R | R | R | R |
| | ONCO II | R | R | R | R | R |
| | SNG12 | NR | R | R | R | R |
| | NMS-P715 | R | NR | R | R | R |
| | CCT251455 | R | R | R | NR | R |
| | Compound 2 | R | R | R | R | NR |
| | Compound 3 | R | R | R | R | NR |

R = resistance
NR = no resistance

### Treatment of Cancer

The present invention provides methods and medical uses for the treatment of MPS1 dysregulated cancer. A cancer may be identified as MPS1 dysregulated cancer by testing a sample of cancer cells from an individual, for example to determine whether a MPS1 kinase inhibitor is capable of killing the cancer cells or reducing the size of a tumour. Examples of cancers known to be treatable in accordance with the present invention include breast, ovarian, thyroid, lung, colon, bladder, haematological and pancreatic cancers and glioblastoma. High levels of MPS1 mRNA expression is known to correlate with a higher histological grade, aggressiveness and poor patient survival in breast cancer, glioblastoma and pancreatic ductal adenocarcinoma (11-17). Furthermore, it has been reported that PTEN-deficient breast cancer cell lines are more sensitive to MPS1 depletion or kinase inhibition (18).

### Detection of Mutations

Mutations described herein are labelled according to the Human Genome Variation Society (http://www.hgvs.org/mutnomen/recs.html). A “p.” preceding the change is used to indicate the mutation is at the protein level. Mutated amino acid residues are described using a one letter code, whereby the first letter indicates the original (wild-type) amino acid at the numbered position in the protein and the latter letter specifies the mutated amino acid. For example, the mutation p.I531M indicates that the MPS1 protein contains a substitution at position 531 of the protein from isoleucine (I) to methionine (M). All protein positions are numbered relative to the human MPS1 amino acid sequence described in SEQ ID NO:1 unless otherwise specified. A “c.” preceding the change is used to indicate the mutation is at the complementary DNA (cDNA) level. Nucleotide substitutions are numbered relative to the human MPS1 nucleotide sequence described in SEQ ID NO:2 unless otherwise indicated and substitutions are indicated with a “>”. For example, the mutation c.1593A>G indicates that the MPS1 DNA contains a substitution at nucleotide position 1593 of the nucleotide sequence from adenine (A) to guanine (G).

Several methods have been developed for the detection of mutations in a sample. The sample may be of normal cells from the individual where the individual has a mutation in the MPS1 gene or the sample may be of cancer cells, e.g. where the cells forming a tumour contain one or more MPS1 mutations. Alternatively, the sample may be a DNA, RNA or protein sample directly obtained from the individual.

When cells are used as the sample, the first step is generally to extract DNA or RNA from the sample. In the case of RNA, mutations can be detected by first carrying out reverse transcription-polymerase chain reaction (RT-PCR) to amplify the cDNA sequence of the target gene. RT-PCR methods have previously been used to determine mutations in the BCR/ABL fusion gene that are associated with resistance to imatinib (54).

Methods for detecting the presence of a mutation in a DNA sample preferably include amplifying at least a portion of the DNA obtained from a sample by PCR using a pair of primers. Primer pairs include a first primer that binds upstream of the target DNA sequence (forward (F) primer) and a second primer that binds downstream of the DNA sequence (reverse (R) primer), such that a portion of the target DNA sequence comprising the mutation is amplified. Preferably, the presence of the mutation can be detected in the amplified DNA or cDNA by direct Sanger sequencing. Additional methods to detect the mutation include matrix-assisted laser desorption/ionization time-of-flight (MALDI-TOF) spectrometry, restriction fragment length polymorphism (RFLP), high-resolution melting (HRM) curve analysis, and denaturing high performance liquid Chromatography (DHPLC). Other PCR-based methods for detecting mutations include allele specific oligonucleotide polymerase chain reaction (ASO-PCR) and sequence-specific primer (SSP)-PCR. Alternatively, the DNA sample can be directly sequenced without an amplification step.

Examples of primers used to amplify the mutations exemplified herein are described in the following table.

| Mutation | F/R | Sequence (5′-3′) | SEQ ID |
|---|---|---|---|
| c.1593A>G | F | GCATTTCGGTTAAAGGAAGAATT | SEQ ID NO: 3 |
| (p.I531M) | | TATTCCA | |
| | R | TTCGTAAATATTTTAAGATACTT | SEQ ID NO: 4 |
| | | ACCTTGCTTGA | |
| c.1831A>G | F | CATCTACATGGTAATGGAGTGTG | SEQ ID NO: 5 |
| (p.S611G) | | GAA | |
| | R | CTTGCGTTCCCATGGATCAATG | SEQ ID NO: 6 |
| c.1799T>C | F | GCAGTGAAATCACGGACCAGTA | SEQ ID NO: 7 |
| (p.M600T) | R | GGATCAATGGATTTTTTCTTTTT | SEQ ID NO: 8 |
| | | AAGCCAACT | |
| c.1703A>G | F | GAACTTAGAAGAAGCAGATAACC | SEQ ID NO: 9 |
| (P.Y5680) | | AAACTCT | |
| | R | GGATGATCTTATCACTGTGTTGT | SEQ ID NO: 10 |
| | | TGTAGT | |
| c.1812T>G | F | GAAATCACGGACCAGTACATCTA | SEQ ID NO: 11 |
| (p.C604W) | | CA | |
| | R | CGTTCCCATGGATCAATGGATTT | SEQ ID NO: 12 |
| | | TT | |

Preferably, small nucleotide polymorphism (SNP) assays are used to detect the mutations in the DNA of cDNA sequences. An example of these assays is droplet digital polymerase chain reaction (ddPCR), a new technology that was recently commercialized to enable the precise quantification of target nucleic acids in a sample. ddPCR measures absolute quantities by counting nucleic acid molecules encapsulated in discrete, volumetrically defined, water-in-oil droplet partitions. This novel ddPCR format offers a simple workflow capable of generating highly stable partitioning of DNA molecules.

In some cases the SNP assays involve the use of allele-specific probes. In this method, each of the allele-specific probes is conjugated to a fluorescent dye which are chosen so that the probe specific for the mutated allele is distinguishable from the probe specific for the wild-type allele. Determining the fluorescence using techniques such as ddPCR allows the quantification of wild-type and mutant alleles. Examples of probes used to detect the mutant (m) and wild-type (wt) alleles exemplified herein are described in the following table.

| Mutation | wt/m | Sequence (5′-3′) | SEQ ID |
|---|---|---|---|
| c.1593A>G | wt | CTCCACTTCCTATCTGC | SEQ ID NO: 13 |
| (p.I531M) | m | CCACTTCCCATCTGC | SEQ ID NO: 14 |
| c.1831A>G | wt | TCTTTTTAAGCCAACTATTAAG | SEQ ID NO: 15 |
| (p.S611G) | m | TTTTAAGCCAACCATTAAG | SEQ ID NO: 16 |
| c.1799T>C | wt | ACACTCCATTACCATGTAGAT | SEQ ID NO: 17 |
| (p.M600T) | m | ACTCCATTACCGTGTAGAT | SEQ ID NO: 18 |
| c.1703A>G | wt | CGTTCCGGTAACTATC | SEQ ID NO: 19 |
| (P.Y568C) | m | TTCCGGCAACTATC | SEQ ID NO: 20 |
| c.1812T>G | wt | AGATCAATATTTCCACACTCC | SEQ ID NO: 21 |
| (p.C604W) | m | AAGATCAATATTTCCCCACTCC | SEQ ID NO: 22 |

Next-generation sequencing (NGS) offers the speed and accuracy required to detect somatic mutations in cancer, either through whole-genome sequencing (WGS) or by focusing on specific regions or genes using whole-exome sequencing (WES) or targeted gene sequencing. Examples of NGS techniques include methods employing sequencing by synthesis, sequencing by hybridisation, sequencing by ligation, pyrosequencing, nanopore sequencing, or electrochemical sequencing.

Fluorescent in situ hybridisation (FISH) is a technique used to detect and localise the presence of specific DNA and RNA sequences. FISH uses fluorescent probes to bind to sequences that show a high degree of complementarity. FISH can be used to identify specific genetic aberrations and to detect the presence or absence of specific cancer biomarkers.

Alternatively or additionally, the present invention the determination of whether a patient has a MPS1 mutated cancer can be carried out by determining whether the MPS1 protein contains one or more mutations. The presence or amount of mutated MPS1 protein may be determined directly using a binding agent, such as an antibody, capable of specifically binding to the mutant MPS1 protein, or fragments thereof. The binding agent may be labelled to enable it to be detected or capable of detection following reaction with one or more further species, for example using a secondary antibody that is labelled or capable of producing a detectable result, e.g. in an ELISA type assay. As an alternative a labelled binding agent may be employed in a Western blot to detect mutant MPS1 protein.

Additionally, the activity of the MPS1 protein may be determined by using techniques well known in the art such as Western blot analysis, immunohistology, chromosomal abnormalities, enzymatic or DNA binding assays and plasmid-based assays. Activity may be determined relative to a control, for example in the case of defects in cancer cells, relative to non-cancerous cells, preferably from the same tissue.

Phosphorylation of MPS1 can be measured as a readout of protein activity. Methods to determine protein phosphorylation include mass spectrometry, and using antibodies specific to the phosphorylated proteins for detection by immunohistochemistry (IHC), immunoblots (Western blots) or ELISA based assays. Phosphorylation can be quantified using an in-cell, fluorescence-based kinase assay using Meso Scale Discovery (MSD) electrochemiluminescence technology as previously described (19).

Furthermore, the functionality of MPS1 can be determined by measuring its kinase activity. Kinase activity assays generally involve isolating the kinase by immunoprecipitation and incubating this kinase with an exogenous substrate in the presence of ATP. The ATP can be labelled for example with a radiolabel (e.g. ATP [γ-33P]). Measurement of the phosphorylated substrate by the target kinase can be assessed by several reporter systems, including colormetric, radioactive or fluorometric detection.

The activity of the MPS1 protein can be determined indirectly by assessing whether the spindle assembly checkpoint (SAC) is functioning correctly. MPS1 is known to be essential for recruitment of the SAC proteins and therefore inhibition of MPS1 can cause cells to prematurely exit the cell cycle (6-10). One method of assessing this is by analysing the cell cycle profiles by flow cytometry. This method generally involves treating cells with a fluorescent dye that stains DNA quantitatively, such as propidium iodide. The intensity of the fluorescence correlates with the amount of DNA and therefore can be used to distinguish cells in different phases of the cell cycle. Furthermore, IHC can be used to identify cells that are in specific phases of the cell cycle, e.g. mitosis. Comparing the cell cycle profiles of different cells can reveal whether there are any cell cycle defects and thus whether the SAC is functioning correctly.

Additionally, the presence of a mutation or mutations in a sample that confers resistance to MPS1 inhibitors can be determined by carrying out cell viability assays. Cell viability assays can be performed using routine methods known to those of skill in the art, such as those described previously (19).

### Gene and Protein Expression

The determination of MPS1 gene expression may involve determining the presence or amount of MPS1 mRNA in a sample. Methods for doing this are well known to the skilled person. By way of example, they include determining and quantifying the presence of MPS1 mRNA (i) using a labelled probe that is capable of hybridising to the MPS1 nucleic acid; and/or (ii) using PCR involving one or more primers based on a MPS1 nucleic acid sequence to determine the amount of MPS1 transcript that is present in a sample. The probe may also be immobilised as a sequence included in a microarray. Levels of mRNA expression may be determined relative to a control, for example in the case of expression in cancer cells, relative to non-cancerous cells, preferably from the same tissue.

Preferably, detecting MPS1 mRNA is carried out by extracting RNA from a sample of the tumour and measuring MPS1 expression specifically using quantitative real time RT-PCR. Alternatively or additionally, the expression of MPS1 could be assessed using RNA extracted from a tumour sample using microarray analysis, which measures the levels of mRNA for a group of genes using a plurality of probes immobilised on a substrate to form the array. The determination of whether the cells are express PTEN and hence are PTEN deficient may be done in an analogous manner.

The determination of MPS1 protein expression can be carried out, for example, to examine whether there are increased levels of MPS1 protein. The presence or amount of MPS1 protein may be determined using a binding agent capable of specifically binding to the MPS1 protein, or fragments thereof. A preferred type of MPS1 protein binding agent is an antibody capable of specifically binding the MPS1 protein or fragment thereof. The antibody may be labelled to enable it to be detected or capable of detection following reaction with one or more further species, for example using a secondary antibody that is labelled or capable of producing a detectable result, e.g. in an ELISA type assay. As an alternative a labelled binding agent may be employed in a Western blot to detect MPS1 protein.

Alternatively, or additionally, the method for determining the presence of MPS1 protein may be carried out on tumour samples, for example using IHC analysis. IHC analysis can be carried out using paraffin fixed samples or frozen tissue samples, and generally involves staining the samples to highlight the presence and location of MPS1 protein.

### Pharmaceutical Compositions

The active agents disclosed herein for the treatment of MPS1 dysregulated cancer may be administered alone, but it is generally preferable to provide them in pharmaceutical compositions that additionally comprise with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. Examples of components of pharmaceutical compositions are provided in Remington's Pharmaceutical Sciences, 20th Edition, 2000, pub. Lippincott, Williams & Wilkins.

Examples of small molecule therapeutics useful for treating MPS1 dysregulated cancer via inhibition of other kinases include: BEZ235, Olaparib and GDC0941.

These compounds or derivatives of them may be used in the present invention for the treatment of MPS1 dysregulated cancer. As used herein “derivatives” of the therapeutic agents includes salts, coordination complexes, esters such as in vivo hydrolysable esters, free acids or bases, hydrates, prodrugs or lipids, coupling partners.

Salts of the compounds of the invention are preferably physiologically well tolerated and non toxic. Many examples of salts are known to those skilled in the art. Compounds having acidic groups, such as phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g., amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques well known in the art.

Derivatives which as prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

The term “pharmaceutically acceptable” as used herein includes compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The active agents disclosed herein for the treatment of MPS1 dysregulated cancer according to the present invention are preferably for administration to an individual in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, Lippincott, Williams & Wilkins. A composition may be administered alone or in combination with other treatments, either simultaneously or sequentially, dependent upon the condition to be treated.

The formulations may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The agents disclosed herein for the treatment of MPS1 dysregulated cancer may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Compositions comprising agents disclosed herein for the treatment MPS1 dysregulated cancer may be used in the methods described herein in combination with standard chemotherapeutic regimes or in conjunction with radiotherapy. Examples of other chemotherapeutic agents include Amsacrine (Amsidine), Bleomycin, Busulfan, Capecitabine (Xeloda), Carboplatin, Carmustine (BCNU), Chlorambucil(Leukeran), Cisplatin, Cladribine(Leustat), Clofarabine (Evoltra), Crisantaspase (Erwinase), Cyclophosphamide, Cytarabine (ARA-C), Dacarbazine (DTIC), Dactinomycin (Actinomycin D),Daunorubicin, Docetaxel (Taxotere), Doxorubicin, Epirubicin, Etoposide (Vepesid, VP-16), Fludarabine (Fludara), Fluorouracil (5-FU), Gemcitabine (Gemzar), Hydroxyurea (Hydroxycarbamide, Hydrea),Idarubicin (Zavedos). Ifosfamide (Mitoxana), Irinotecan (CPT-11, Campto), Leucovorin (folinic acid), Liposomal doxorubicin (Caelyx, Myocet), Liposomal daunorubicin (DaunoXome®) Lomustine, Melphalan, Mercaptopurine, Mesna, Methotrexate, Mitomycin, Mitoxantrone, Oxaliplatin (Eloxatin), Paclitaxel (Taxol), Pemetrexed (Alimta), Pentostatin (Nipent), Procarbazine, Raltitrexed (Tomudex®), Streptozocin (Zanosar®), Tegafur-uracil (Uftoral), Temozolomide (Temodal), Teniposide (Vumon), Thiotepa, Tioguanine (6-TG) (Lanvis), Topotecan (Hycamtin), Treosulfan, Vinblastine (Velbe), Vincristine (Oncovin), Vindesine (Eldisine) and Vinorelbine (Navelbine).

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound, and so the actual weight to be used is increased proportionately.

### Experimental Examples

### Methods

### Cell Culture and Molecular Cell Biology

All cells were cultured in DMEM, supplemented with 10% foetal calf serum, 2 mM L-glutamine, 100 U/ml penicillin and 100 μg/ml streptomycin. Stably transfected, tetracycline-inducible DLD1 Flp-In T-Rex cells were created as previously described (32). For cell viability assays, 2000 cells were plated per well and assessed using CellTiterGlo Luminescent Cell Viability Assay after 4 days (Promega). For colony formation assays, 500 cells were plated per well and analysed using Sulforhodamine B colourimetric assay after 14 days (SRB; Sigma). Total mRNA was extracted from cells using RNeasy Mini kit (Qiagen) and MPS1 cDNA amplified using ImProm-II Reverse transcription protocol (Promega). Site directed mutagenesis was performed using QuickChange II (Agilent Technologies). Tetracycline (Sigma) was used at a final concentration of 1 μg/ml, Nocodazole (Sigma) at 200 ng/ml, Paclitaxel (Sigma) at 200 nM and MG132 (Sigma) at 20 μM.

### IP-Kinase Assays

Myc-tagged MPS1 constructs were transfected into HEK293T cells (ATCC), the cells arrested in nocodazole and lysed in lysis buffer (Cell Signaling). Myc-MPS1 was captured using 7 μg of anti-myc antibody (4A6: Millipore, 05-724) coupled to Protein G Dynabeads (Life Technologies), being re-suspended in 18 μl kinase buffer. 15 μl of the IP was then incubated with 10 μg MBP (Sigma), 166 mM ATP (sigma) and 5 μCi ATP [γ-33P] (PerkinElmer) for 30 min at 30° C. The reactions were stopped by addition of SDS loading buffer and boiling at 100° C. for 5 min, then the samples run on NuPAGE Tris-Acetate gels (Life Technologies). The gels were stained with SimplyBlue Safestain (Life Technologies) and radioactivity quantified using a 9410 Typhoon phosphorimager and ImageQuant software (Amersham Biosciences).

### Immunofluorescence and Time-Lapse Microscopy

For analysis by immunofluorescence, cells were fixed in 1% formaldehyde for 5 min at room temperature, quenched in glycine, washed in PBS-Triton X-100 (0.1% PBS-T) and incubated for 1 hour in primary antibodies in PBS-T: MAD2 (Bethyl Laboratories Inc., A300-301A), CDC2020 (Millipore, MAB3775), MPS1 (Millipore, 05-682), MPS1 pT33pS37 (Life Technologies, 44-1325G) and ACA (ImmunoVision, HST-0100). After PBS-T washes, cells were incubated with fluorescent-conjugated secondary antibodies (Life Technologies), stained with DAPI (Life Technologies) and mounted onto slides with Vectashield (Vector Labs). Images were acquired using a Zeiss LSM 710 confocal microscope and processed using Velocity 3D Image analysis software (PerkinElmer). Time-lapse micrscopy was performed in 96-well Ibidi plate (Thistle Scientific) using a Diaphot inverted microscope (Nikon), in a humidified CO₂ chamber at 37° C., using a motorized stage (Prior Scientific), controlled by Simple PCI software (Compix).

### Flow Cytometry

Cells were fixed overnight at −20° C. in 70% ethanol, washed in PBS, then incubated in 10 μg/ml propidium iodide and 0.5% RNase (Sigma) for 30 min and then analysed using LSRII flow cytometer (BD Biosciences). To stain for mitosis, cells were incubated for 1 hour at 4° C. with anti-MPM2 antibodies (Millipore, 05-368), then 1 hour at 4° C. with FITC-conjugated secondary antibodies (Life technology).

### Meso Scale Discovery (MSD) Assay

Cellular IC50 values for MPS1 pS33pT37auto-phosphorylation inhibition were measured as previously described (19).

### Droplet Digital PCR

Droplet digital PCR was carried out utilizing a QX100 droplet digital PCR system (Bio-Rad) and TagMan MGB primer-probes (Applied biosystems, supplementary). DNA was extracted from cell lines using DNeasy blood and tissue kit (Qaigen). All tumour and lymphoblast samples were fresh frozen. PCR reactions were carried out using 10 it Supermix buffer (Bio-Rad) and 1 μl of primer-probes mix (Life Technologies), then an emulsion made using droplet oil in the QX100 droplet-generator (Bio-Rad). PCR reactions were then carried out on a thermal cycler at: 95° C. for 10 min, 40 cycles of 95° C. for 15 s and 57.5-63.5° C. for 1 min, then 10 min at 98° C. Plates were analysed on a Bio-Rad QX100 droplet reader using QuantaSoft software. Fraction abundances (FA %) were calculated as: [a/(a+b)]×100, where a: is the total number of mutant-positive droplets, and b: is the total number of wild-type positive droplets.

### Protein Production and Purification and Crystal Structure Determination

The MPS1-KD wild-type and mutant proteins were produced as previously described (19, 49). For protein expression of full-length MPS1 proteins, Sf9 insect cells were grown at 27° C. in sf-900 II media (Life Technologies) to a cell density of around 2×10⁶ cells/mL and infected with sufficient virus to cause cessation of cell growth within 24 hours, typically 30 μL to 100 μL of virus per 10′ cells. Infected cell cultures were harvested (6,238×g, 4° C., 20 min) 3 days post infection. Cell pellets were resuspended in 3 volumes of Lysis Buffer (50 mM HEPES pH 7.4, 100 mM NaCl, 1 mM MgCl₂ and 10% (v/v) glycerol) containing 1× cOmplete™ EDTA-free protease inhibitors (Roche), 20 mM β-glycerophosphate, 10 mM NaF, 2 mM Na₃VO₄ and 25 U/mL Benzonase® nuclease (Merck Chemicals Ltd) prior to lysis by sonication using a Vibra-Cell™ VCX500 (Sonics & Materials Inc.) with a 13 mm solid probe at 50% amplitude in 5 s bursts. The lysate was clarified by centrifugation (75,600×g, 10° C., 45 min) and the supernatant was purified over 10 mL of Talon® resin (Clontech) using a batch/gravity protocol, washing with 30 column volumes (CV) of Wash Buffer (50 mM HEPES pH 7.0, 300 mM NaCl and 10% (v/v) glycerol) and eluting with 5 CV Talon Elution Buffer (Wash Buffer including 250 mM imidazole and 1× cOmplete™ EDTA-free protease inhibitors). The eluate from the Talon® column was subsequently applied to a 5 mL GSTrap™ FF column (GE Healthcare) equilibrated in Wash Buffer. After washing with 10 CV of Wash Buffer, the protein was eluted with 4 CV GSH elution buffer (75 mM Tris pH 7.5, 300 mM NaCl, 50 mM glutathione, 2 mM DTT, 1 mM EDTA and 0.002% (v/v) Triton™ X-100). Eluted protein was subsequently dialysed overnight against 50 mM Tris pH 7.5, 150 mM NaCl, 1 mM DTT, 0.5 mM EDTA, 0.01% (v/v) Triton™ X-100 and 50% (v/v) glycerol), snap frozen in liquid nitrogen in aliquots, and stored at −80° C.

All crystallisation experiments were performed at 18° C. by the sitting drop vapour diffusion technique. Soaks were also carried out at 18° C. For co-crystallisation experiments, pre-incubations of protein with ligands were performed for 30 minutes on ice prior to setting up crystallisation plates.

### Antibodies

The following antibodies were used for immunoblotting: anti-GFP (Clonetech, 632381), MPS1 (Millipore, 05-682), α-tubulin (Sigma, T9026), MPM2 (Millipore, 05-368), MPS1 pT33pS37 (Life Technologies, 44-1325G), MPS1 pT676(1), and MYC (Millipore, 05-724). The following antibodies were used for immunofluorescence: anti-BUB1 (Abcam, ab54893), BUBR1 (BD Biosciences, 612503), MPS1 pT676(1), MAD1 (Abcam, ab45286), ZW10 (Abcam), ZWINT-1 (Abcam, ab84367), CENP-F (Abcam, ab90), CENP-E (Abcam, ab5093), CENP-A pS7 (New England Biolabs, 21875) and ACA (Immunovision, HST-0100).

### Sequences of Primers and Probes

MPS1 reverse transcription was performed using primers 5′-CGGATCCGAATCCGAGGATTTAAGTGGC-3′ (SEQ ID NO: 23) and 5′-CACGCGGCCGCTCATTTTTTTCCCCTTTTTTTTTC-3′ (SEQ ID NO: 24), to clone into a modified pcDNA5/FRT/TO-GFP and -Myc vectors.

Site directed mutagenesis was performed using primers:

I531M
(SEQ ID NO: 25)
5′-CCATATTAAAGCAGATGGGAAGTGGAGGTTCAAGC
and
(SEQ ID NO: 26)
5′-GCTTGAACCTCCACTTCCCATCTGCTTTAATATGG;
S611G:
(SEQ ID NO: 27)
5′GGAAATATTGATCTTAATGGTTGGCTTAAAAAG
and
(SEQ ID NO: 28)
5′-CTTTTTAAGCCAACCATTAAGATCAATATTTCC;
M600T
(SEQ ID NO: 29)
5′-CGGACCAGTACATCTACACGGTAATGGAGTGTGG
and
(SEQ ID NO: 30)
5′-CCACACTCCATTACCGTGTAGATGTACTGGTCCG;
Y568C
(SEQ ID NO: 31)
5′CCAAACTCTTGATAGTTGCCGGAACGAAATAGC
and
(SEQ ID NO: 32)
5′-GCTATTTCGTTCCGGCAACTATCAAGAGTTTGG;
C604W
(SEQ ID NO: 33)
5′-GGTAATGGAGTGGGGAAATATTGATCTTAATAGTTGGC
and
(SEQ ID NO: 34)
5′-GCCAACTATTAAGATCAATATTTCCCCACTCCATTACC,
for the sense and anti-sense strands respectively.

S611G and C604W mutations were also introduced into the modified pFastBac1 vector bearing the coding sequence for full length MPS1, as described previously (19), as well as a plasmid for expression of the MPS1 kinase domain (residues 519-808), kindly provided by Stephan Knapp (Structural Genomics Consortium, Oxford, UK). Recombinant baculovirus used in the expression of full-length MPS1 were generated according to Bac protocols (Life Technologies). For ddPCR reactions, custom made primer-probes were designed by Life Technologies, assay numbers: AHCS5N3 for MPS1 p.S611G, AHCS7V2 for MPS1 p.I531M, AHFA38F for MPS1 p.M600T, AHD1517 for MPS1 p.Y568C, AHGJ2EN for MPS1 p.C604W, AHQJQA4 for p.S611R, AHRSOHC for S611C, AHN1TY0 for Y568Stop and AHLJOAV for EGFR p.T790M.

### Recombinant MPS1 Kinase Assays

The enzyme activities of recombinant wild-type and mutant MPS1 proteins were assayed with an electrophoretic mobility shift assay as described previously (19) with the following minor modifications. The protein concentrations used were as follows: wild-type MPS1 (6 nM), p.S611G (12.5 nM) and p.C604W (100 nM). For the low ATP concentration assays, the concentration of ATP used was the same as the Kₘ value for the respective MPS1 protein as shown in Table 2 below. For high ATP concentration assays, 1 mM ATP was used. An ECHO® 550 (Labcyte Inc) acoustic dispenser was used to generate duplicate 8 point dilution curves directly into 384-well low-volume polystyrene assay plates (Corning Life Sciences). The reaction was carried out for 90 min at room temperature.

### Preparation of Compound 1,2 and 3

Preparation of compound 1 has been described (19). The synthesis of compound 2 is described in patent WO 2012/123745 A1.

Chemically, compound 3 is named isopropyl 6-(4-(1,2-dimethyl-1H-imidazol-5-yl)-2-fluorophenylamino)-2-(1-methyl-1H-pyrazol-4-yl)-1H-pyrrolo[3,2-c]pyridine-1-carboxylate and has the structure:

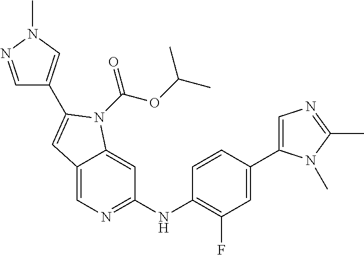 [SMILES: Cc1ncc(-c2ccc(Nc3cc4c(cn3)cc(-c3cnn(C)c3)n4C(=O)OC(C)C)c(F)c2)n1C]

In order to synthesise compound 3, 4-(1,2-Dimethyl-1H-imidazol-5-yl)-2-fluoroaniline was prepared:

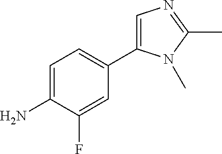

Tetrakis(triphenylphosphine)palladium (48.7 mg, 0.042 mmol) was added to a solution of 2-fluoro-4-(4,4,5,5-tetramethyl-1,3,2-dioxaborolan-2-yl)aniline (100 mg, 0.422 mmol), 5-bromo-1,2-dimethyl-1H-imidazole (81 mg, 0.464 mmol) and cesium fluoride (192 mg, 1.265 mmol) in DME/MeOH 2/1 (2.6 mL). The reaction mixture was heated for 10 min at 150° C. under microwave irradiation. It was then diluted with EtOAc and quenched with water. The layers were separated and the aqueous layer was extracted with EtOAc. The combined organic layers were dried (Na₂SO₄), filtered and concentrated under reduced pressure. The crude mixture was filtered on SCX-2 column and was then purified by Biotage column chromatography (1 to 2% MeOH/aq. NH₃ (10/1) in EtOAc; 12 g column) to afford the title product as a white solid (62 mg, 72%).

¹H NMR (500 MHz, CDCl₃) 2.42 (s, 3H), 3.48 (s, 3H), 3.88 (br s, 2H), 6.81 (dd, J=9.2, 8.1 Hz, 1H), 6.86 (s, 1H), 6.92 (ddd, J=8.1, 1.9, 0.8 Hz, 1H), 6.98 (dd, J=11.8, 1.9 Hz, 1H); LC (Method B) −MS (ESI, m/z) t_{R} 0.57 min, 206 [(M+H⁺), 100%].

For the synthesis of compound 3:

Tris(dibenzylideneacetone)dipalladium(0) (5.0 mg, 5.51 μmol) was added to a mixture of isopropyl 6-bromo-2-(1-methyl-1H-pyrazol-4-yl)-1H-pyrrolo[3,2-c]pyridine-1-carboxylate (19) (0.04 g, 0.110 mmol), cesium carbonate (0.072 g, 0.220 mmol), 4-(1,2-dimethyl-1H-imidazol-5-yl)-2-fluoroaniline (0.025 g, 0.121 mmol) and xantphos (6.4 mg, 0.011 mmol) in DMA (1.2 mL). The reaction mixture was heated at 70° C. for 1.5 h. It was then filtered on SCX-2 column and concentrated under vacuum. The residue was purified by Biotage column chromatography (1 to 5% MeOH/aq. NH₃ (10/1) in EtOAc, 12 g column) to afford the title product as a yellow solid (35 mg, 65%).

¹H NMR (500 MHz, CDCl₃) 1.33 (d, J=6.3 Hz, 6H), 2.46 (s, 3H, CH₃), 3.55 (s, 3H), 3.97 (s, 3H), 5.19 (sept, J=6.3 Hz, 1H), 6.54 (d, J=0.9 Hz, 1H), 6.79 (d, J=3.0 Hz, 1H), 6.95 (s, 1H), 7.10-7.15 (m, 2H), 7.57 (d, J=0.7 Hz, 1H), 7.63 (d, J=0.7 Hz, 1H), 7.66 (t, J=0.9 Hz, 1H), 8.08 (t, J=8.6 Hz, 1H), 8.49 (d, J=0.9 Hz, 1H); LC (Method A) −MS (ESI, m/z) t_{R} 1.62 min, 202 [(M-C₃H₇O₂+2H⁺²), 100%]; ESI-HRMS (Method B) Found 488.2202, calculated for C₂₆H₂₇FN₇O₂ (M+H⁺): 488.2205.

### Results

### The Generation of AZ3146-Resistant Cell Lines

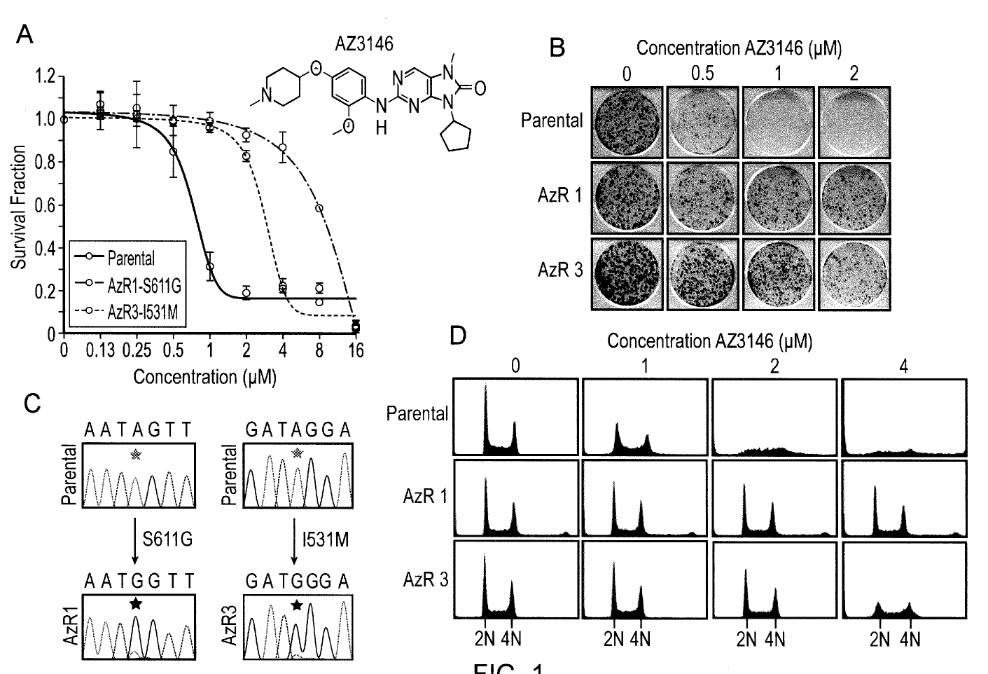

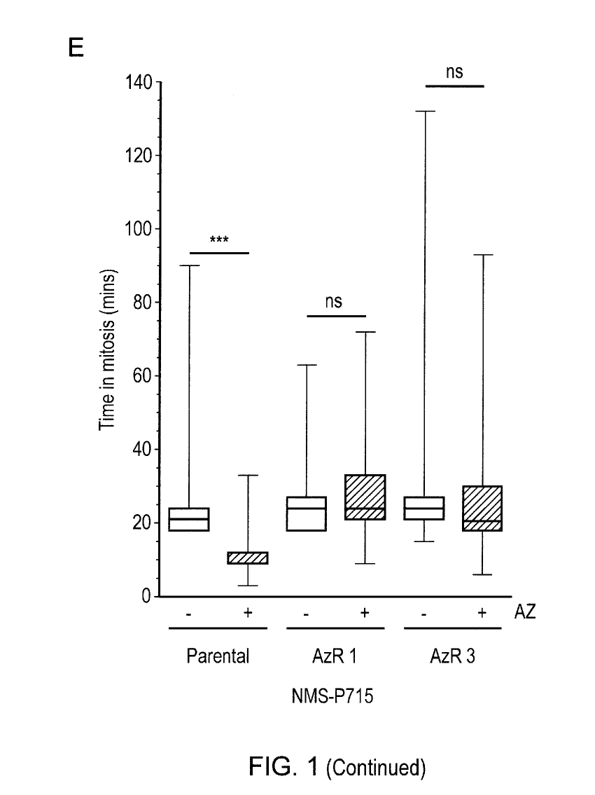

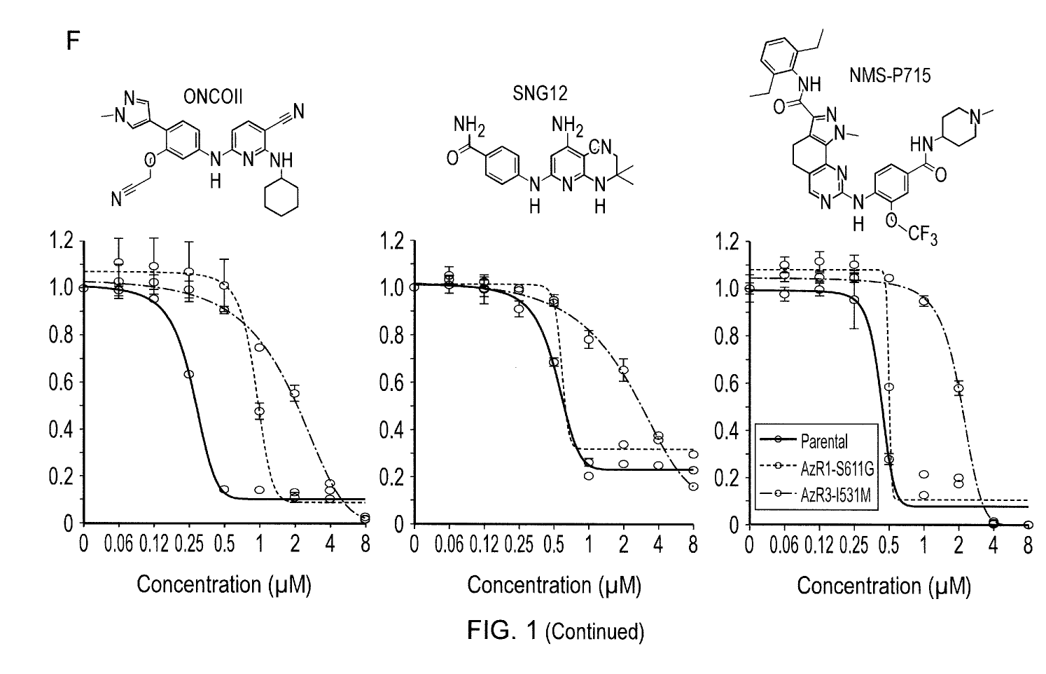

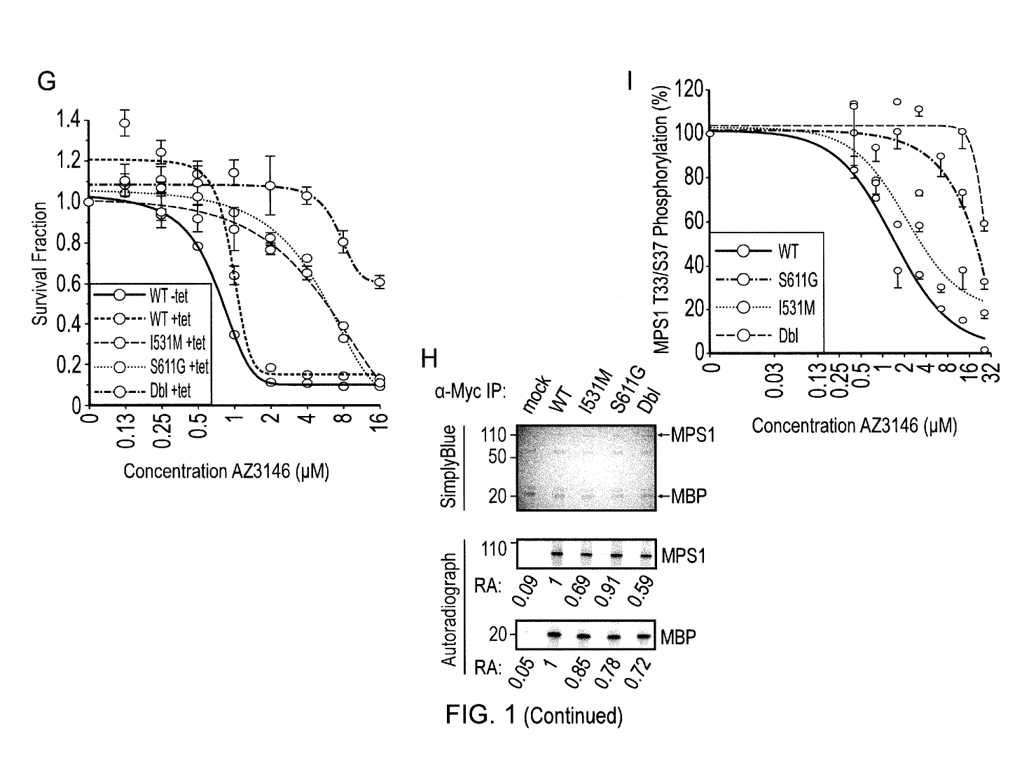

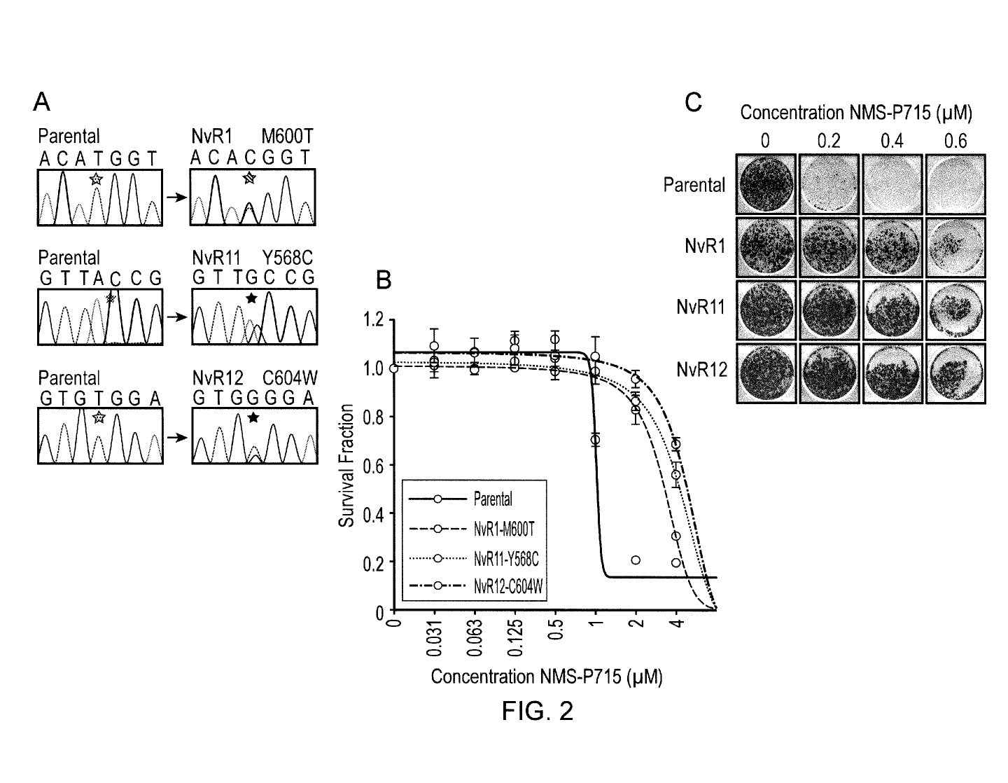

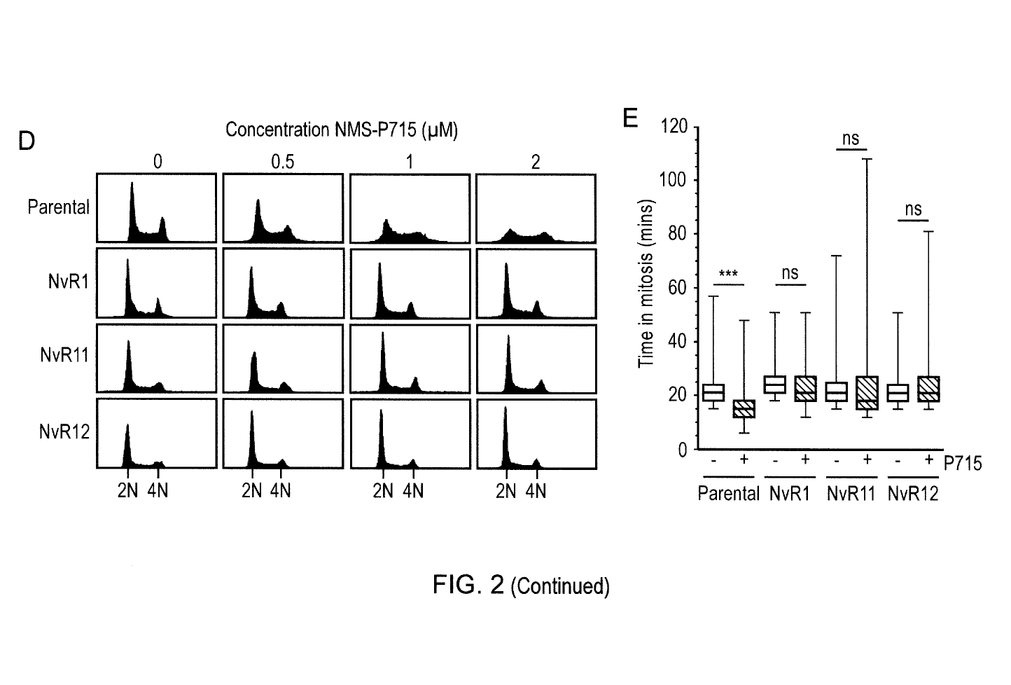

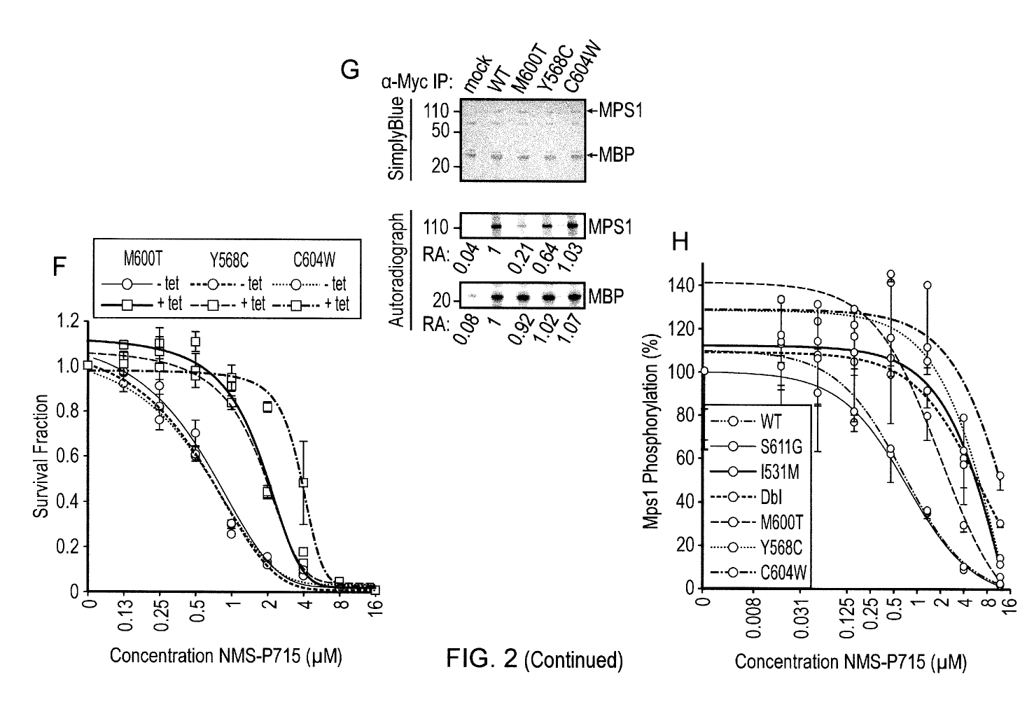

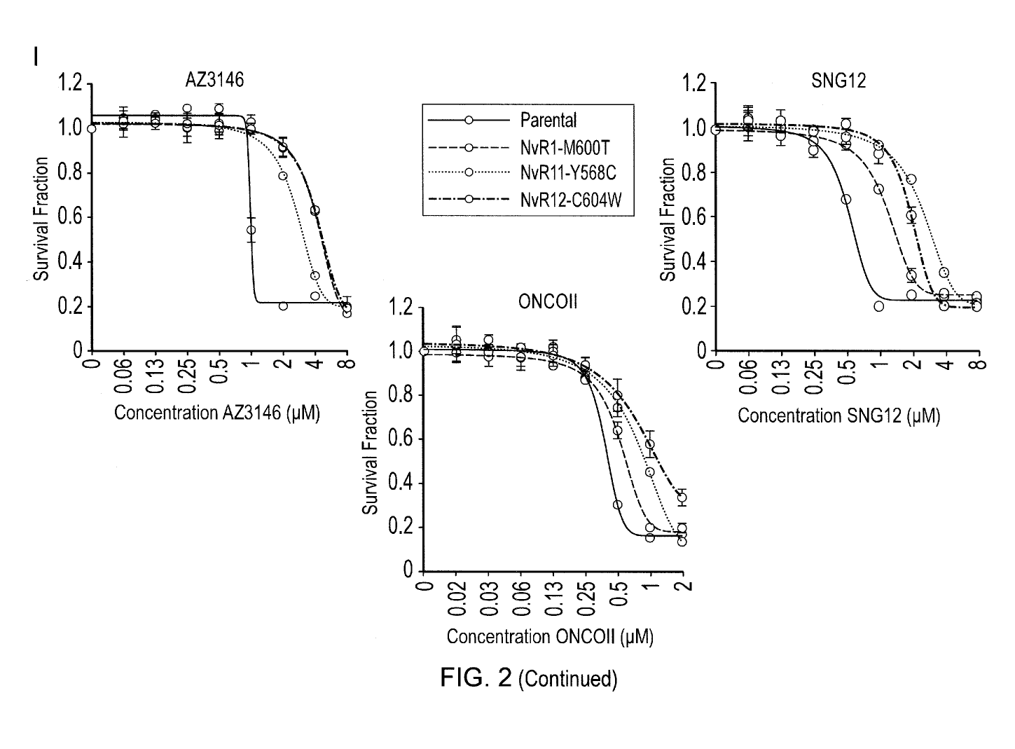

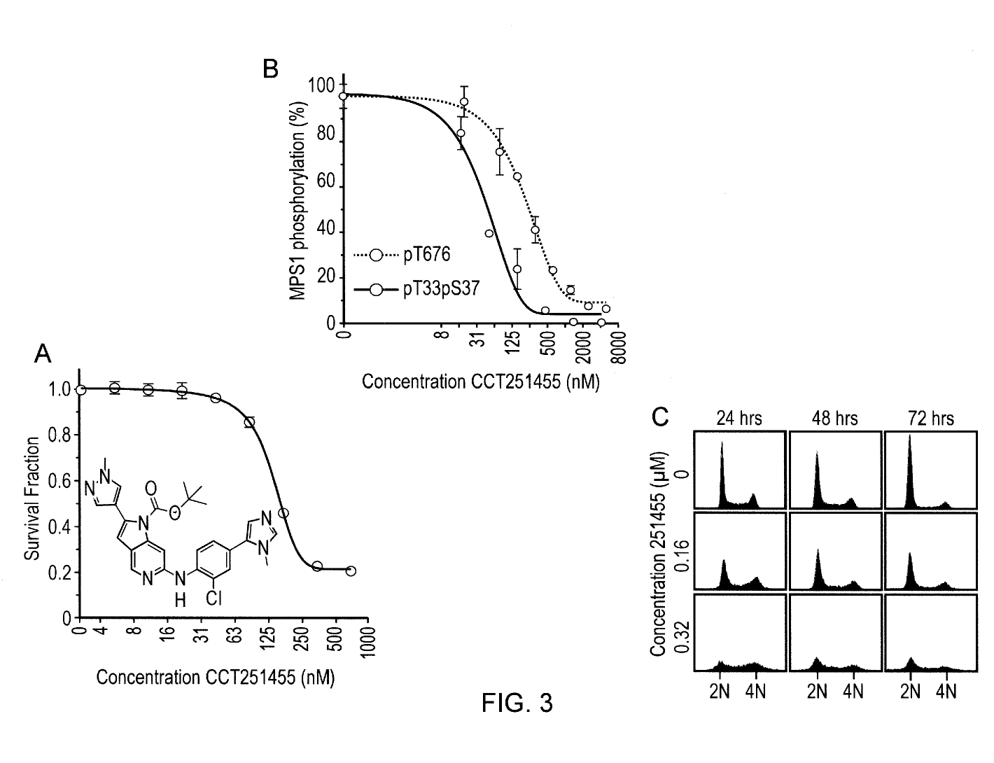

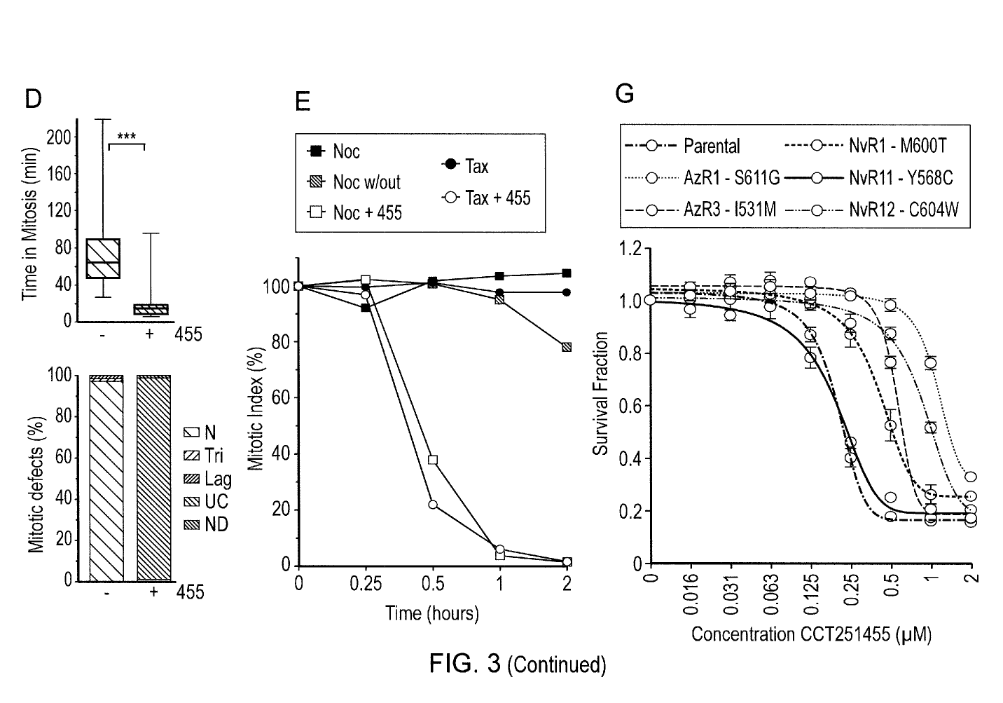

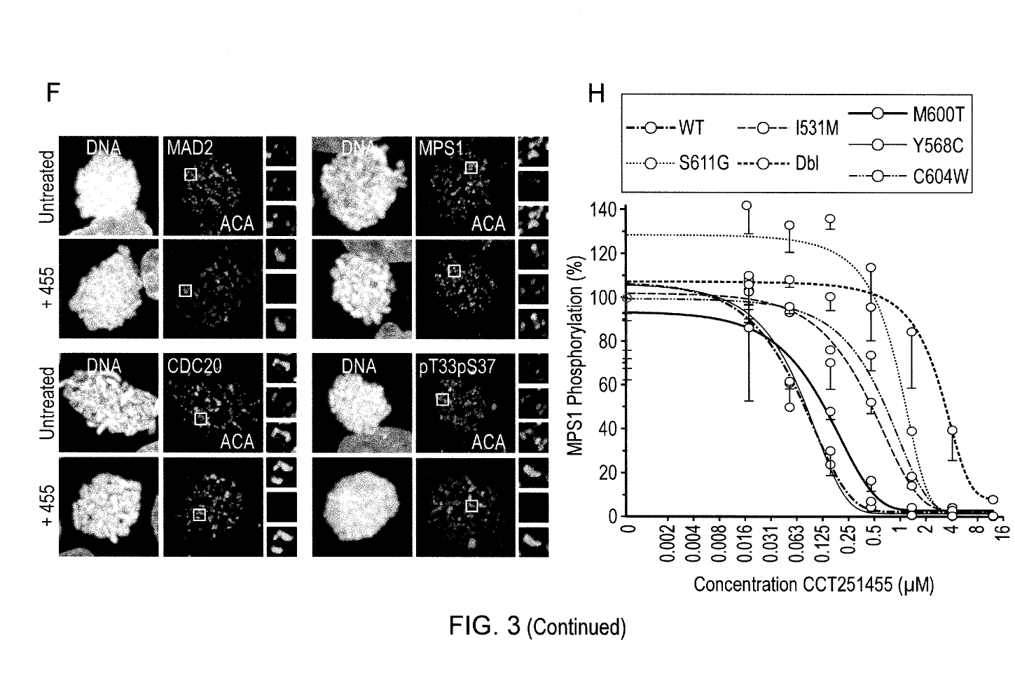

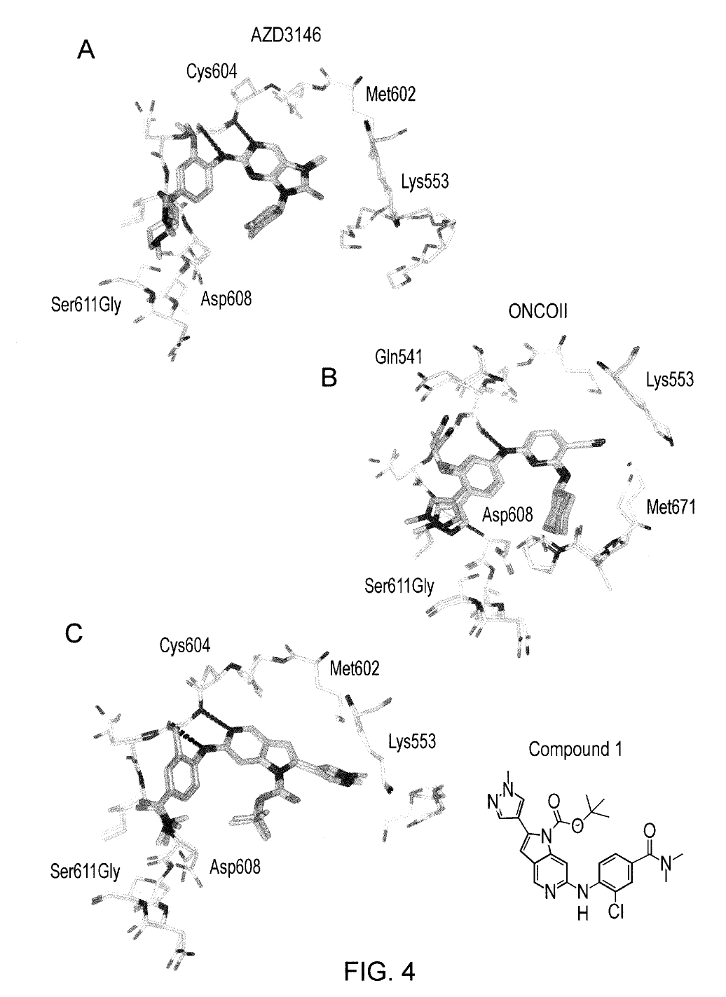

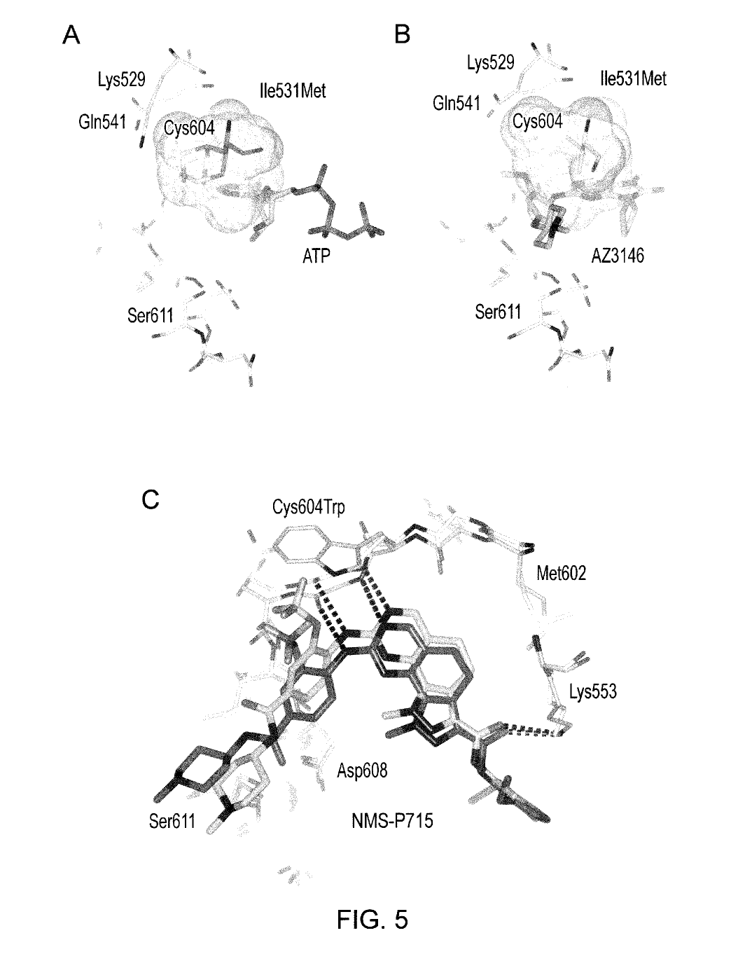

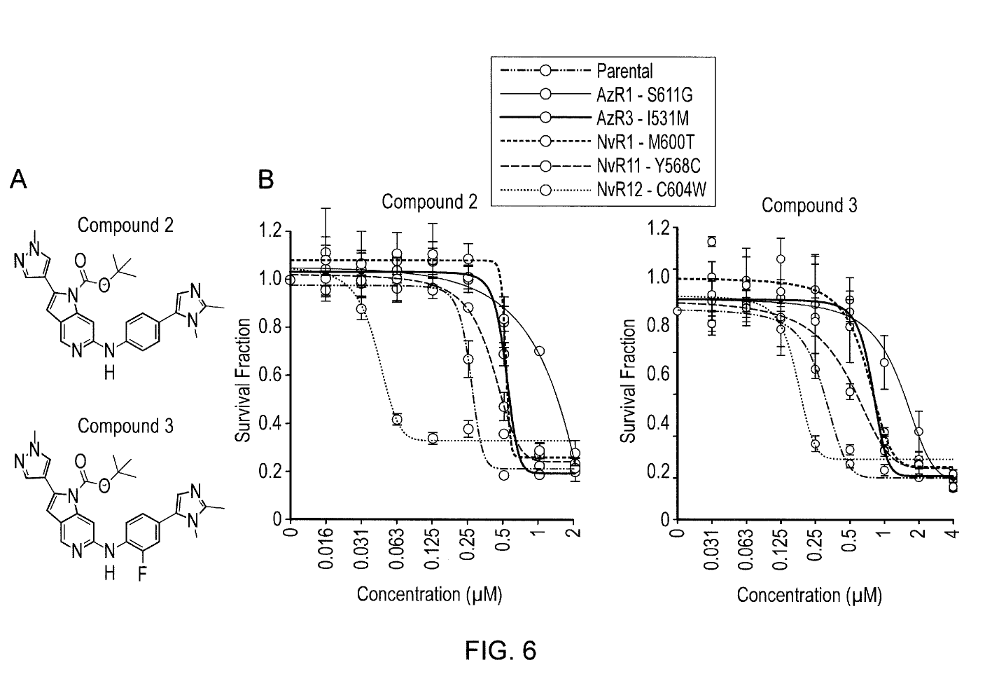

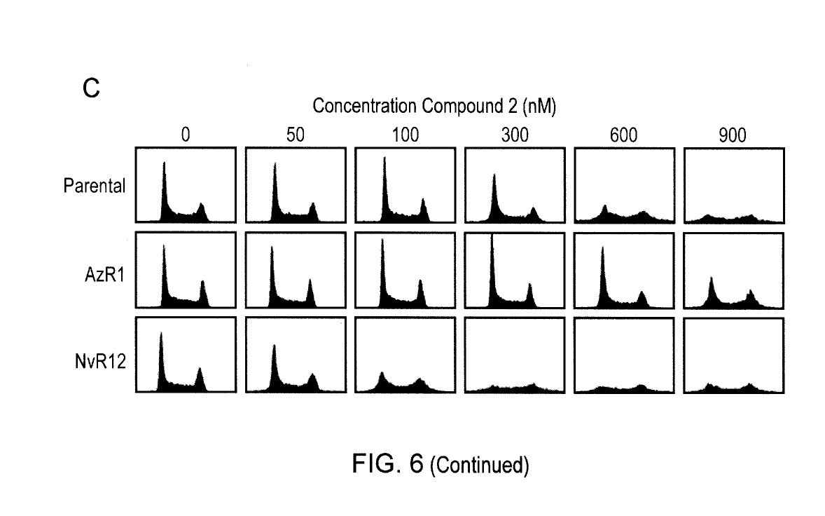

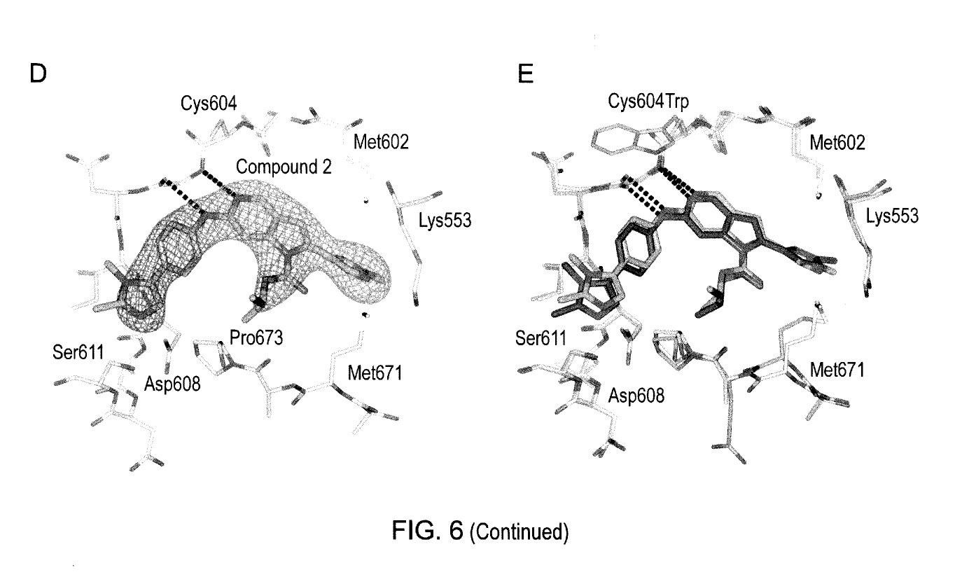

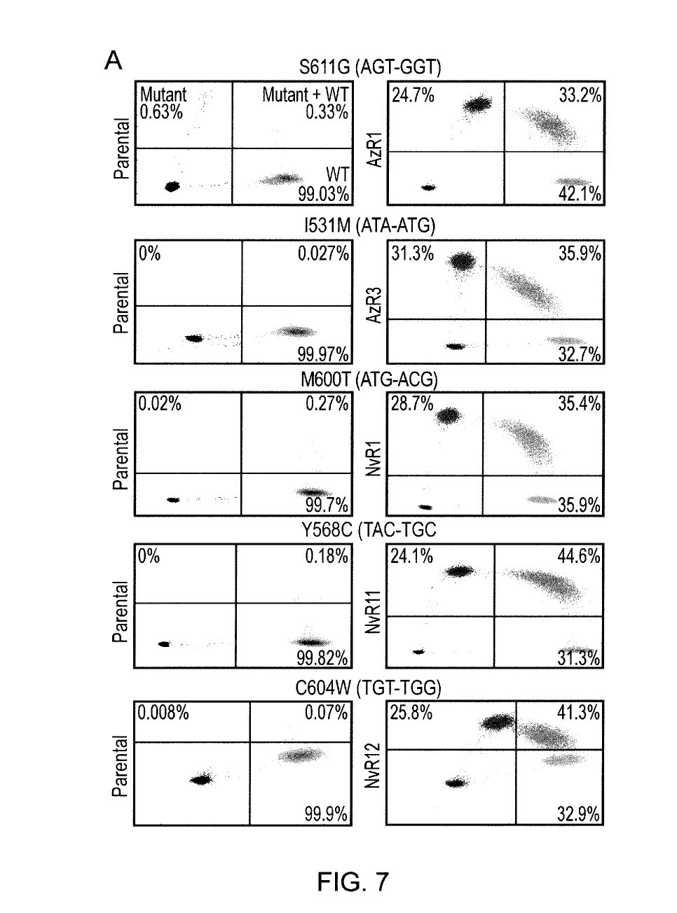

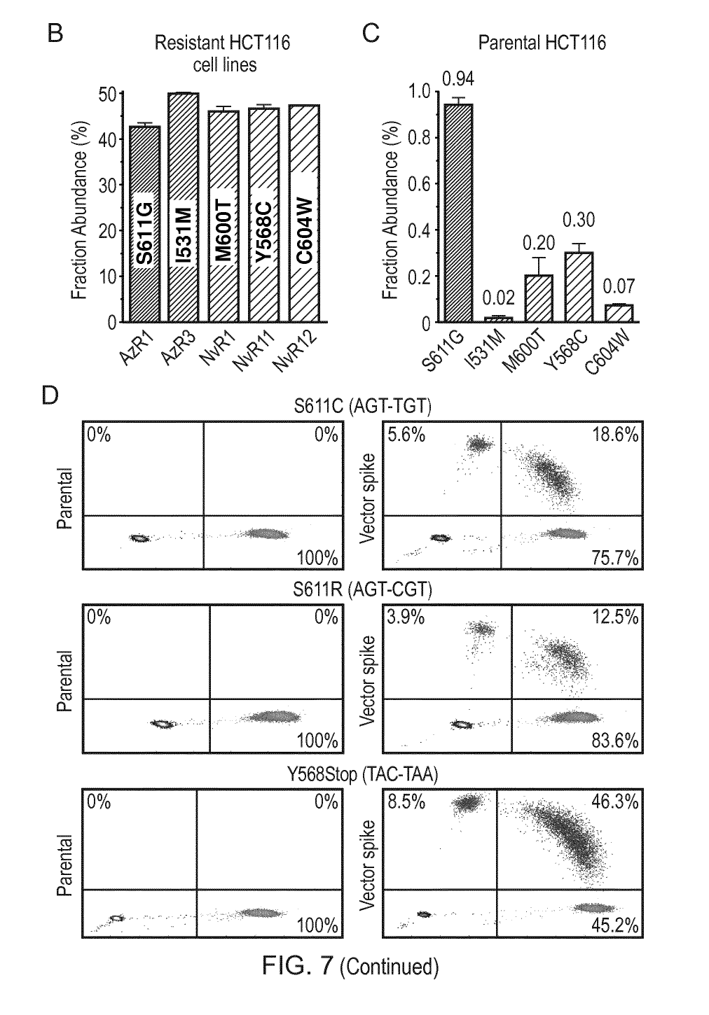

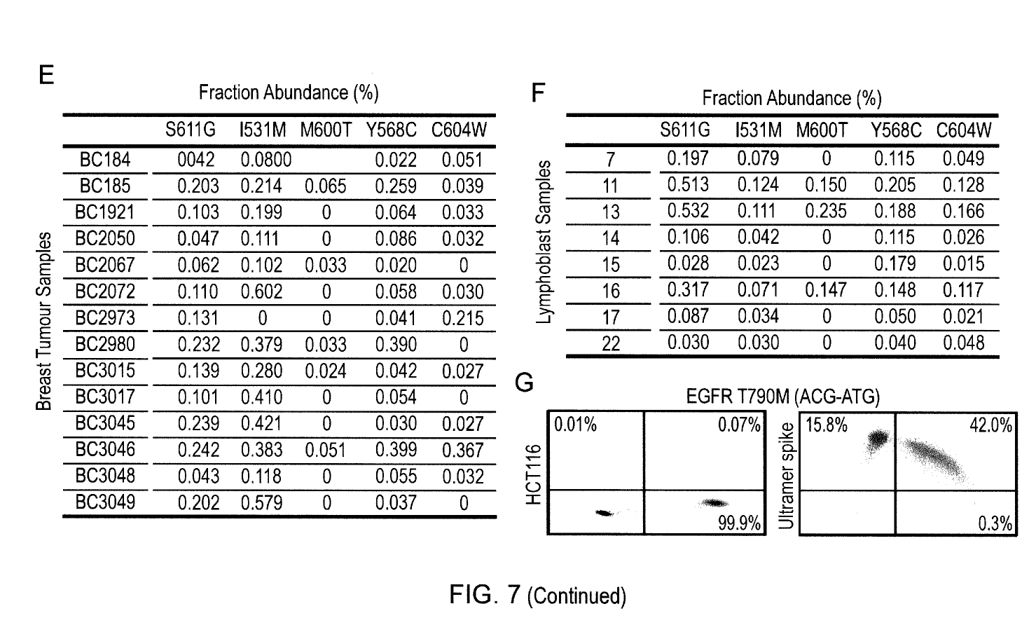

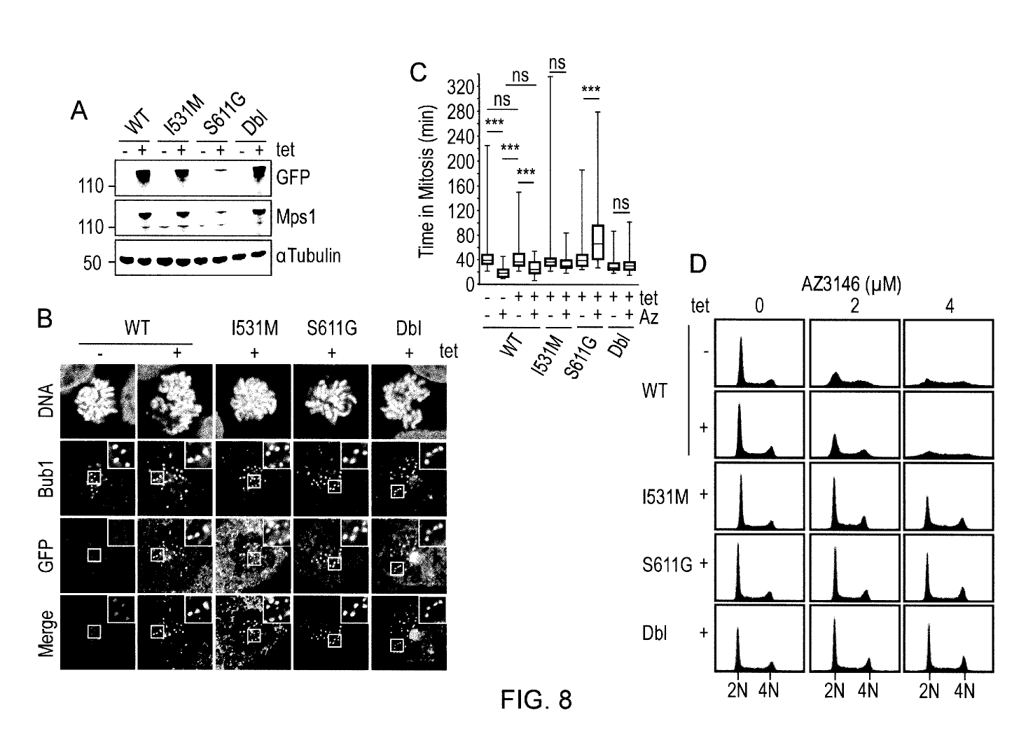

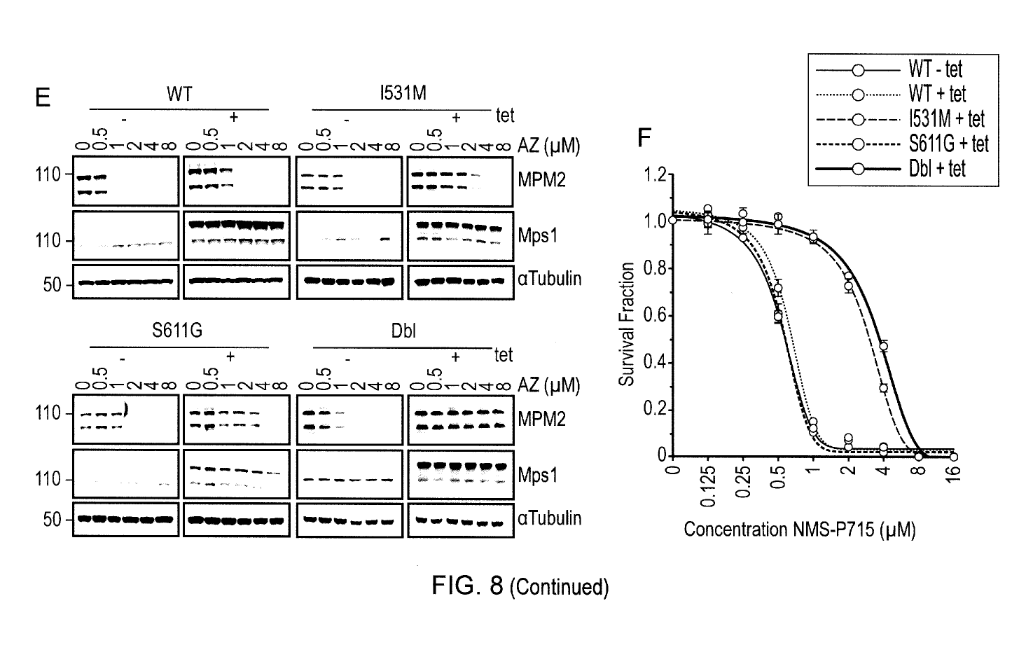

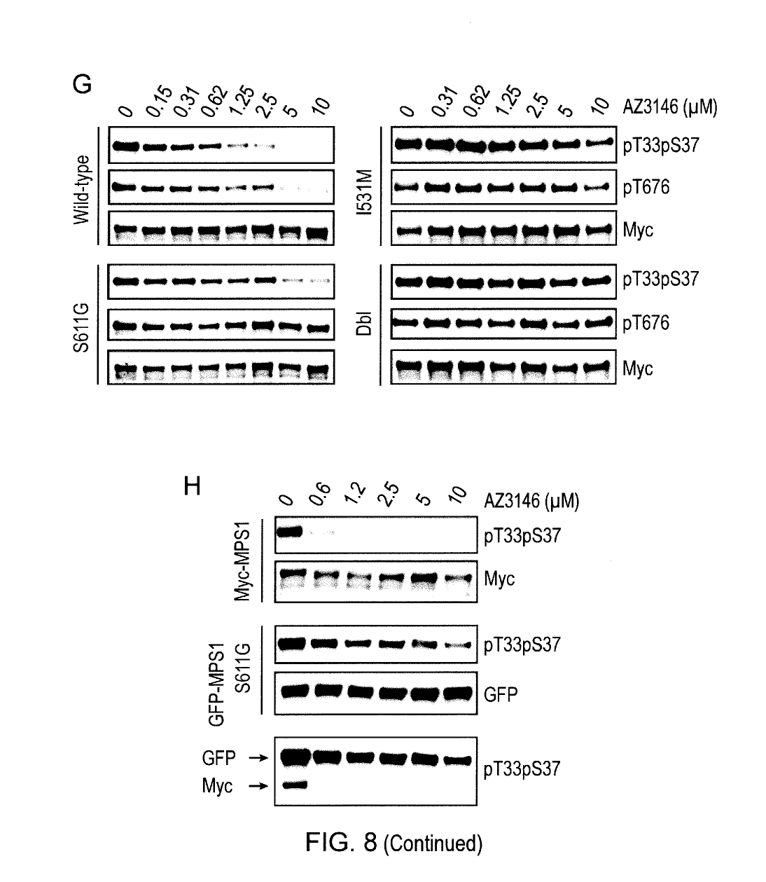

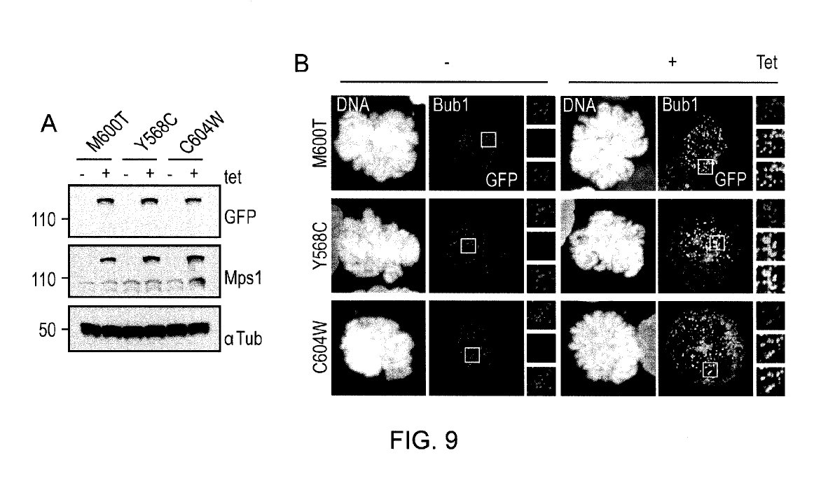

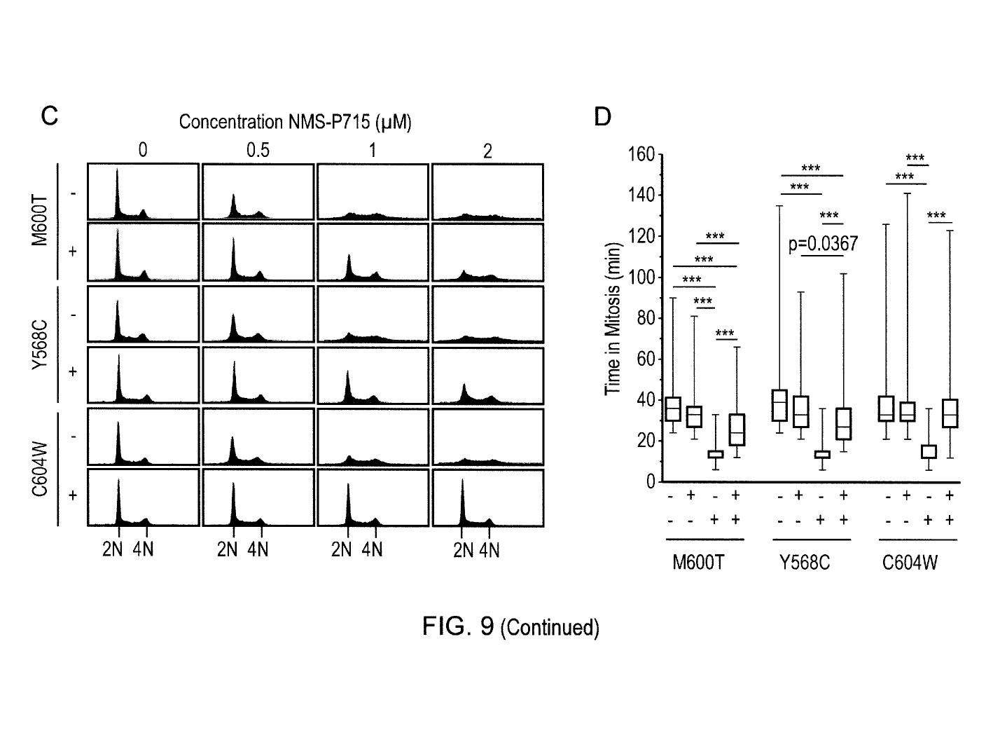

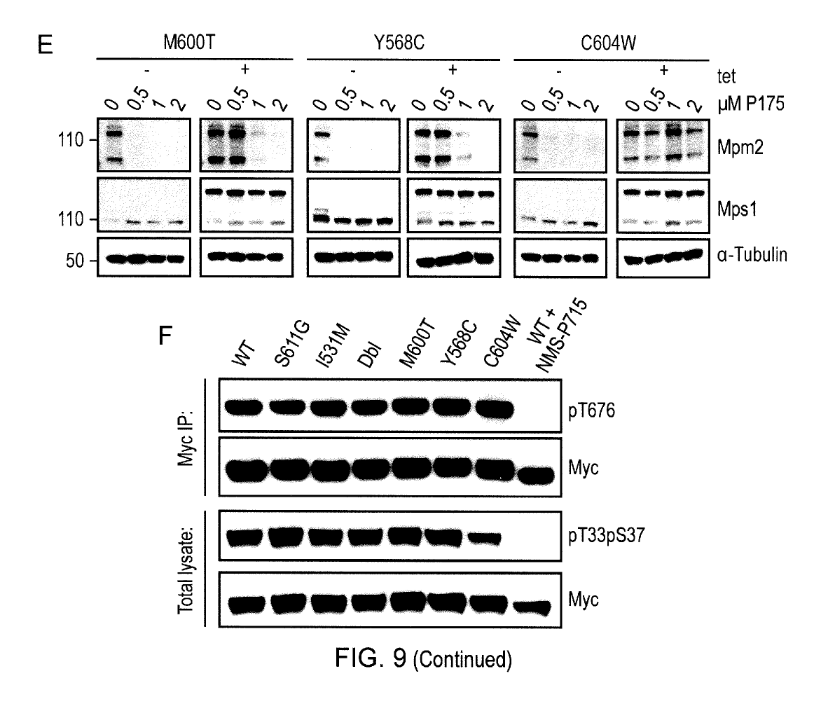

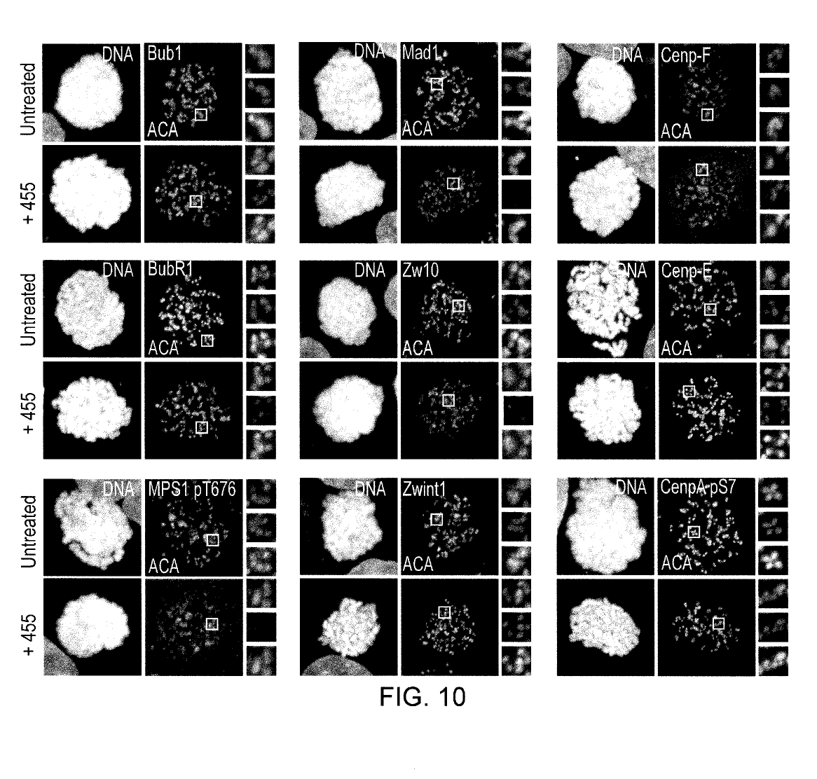

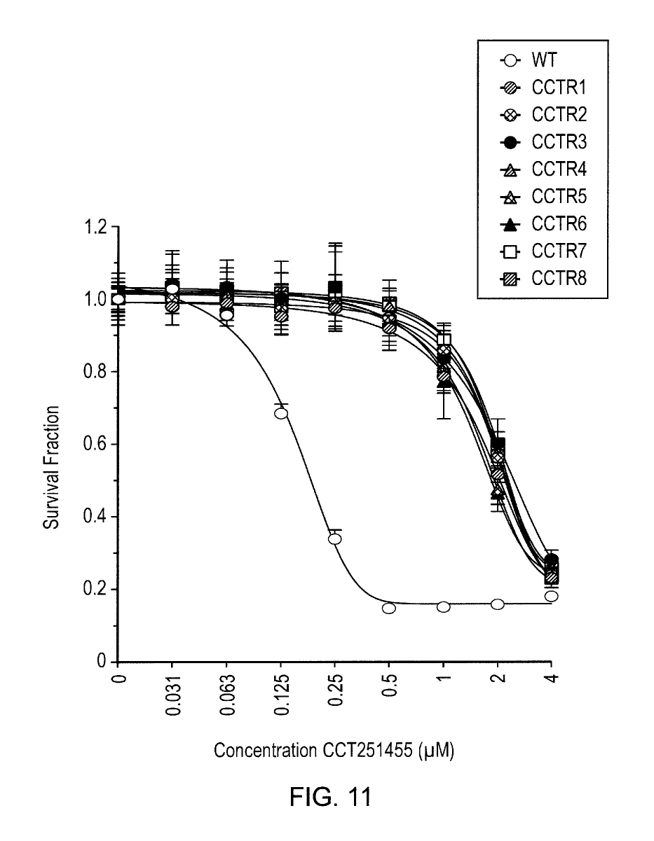

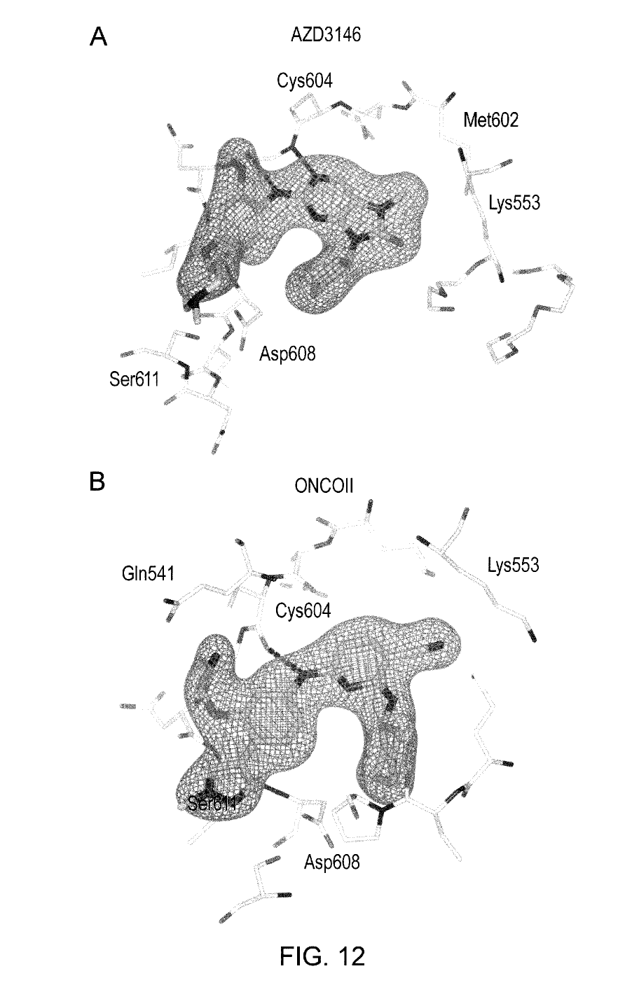

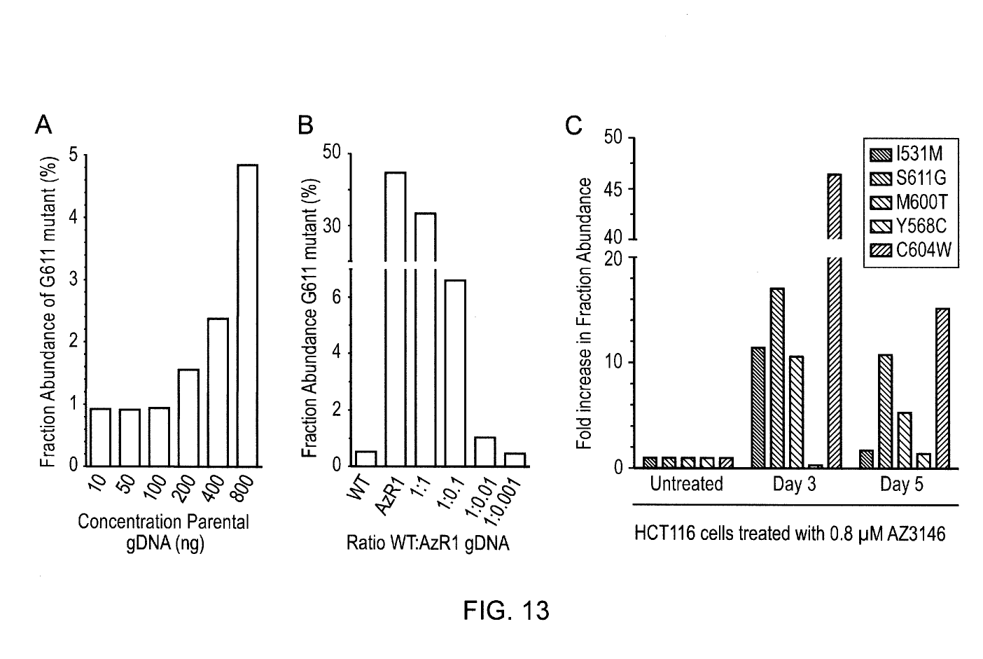

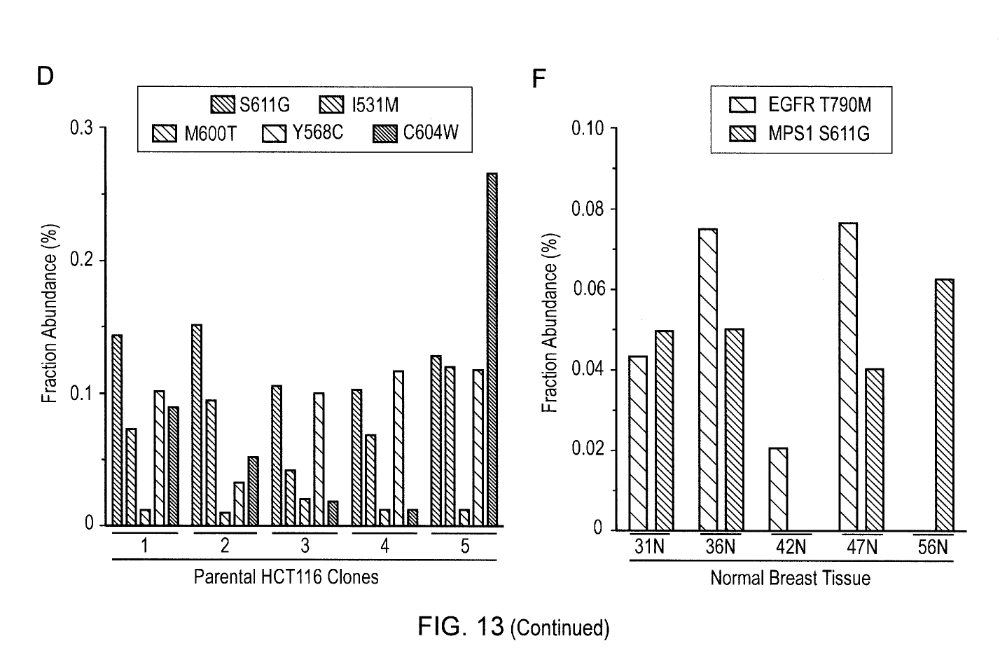

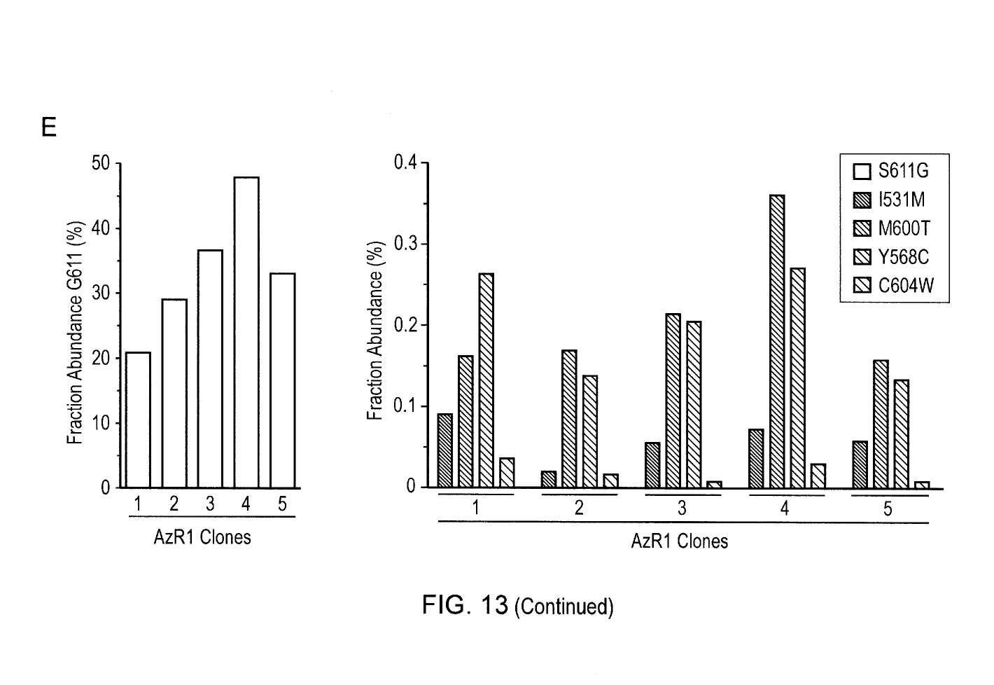

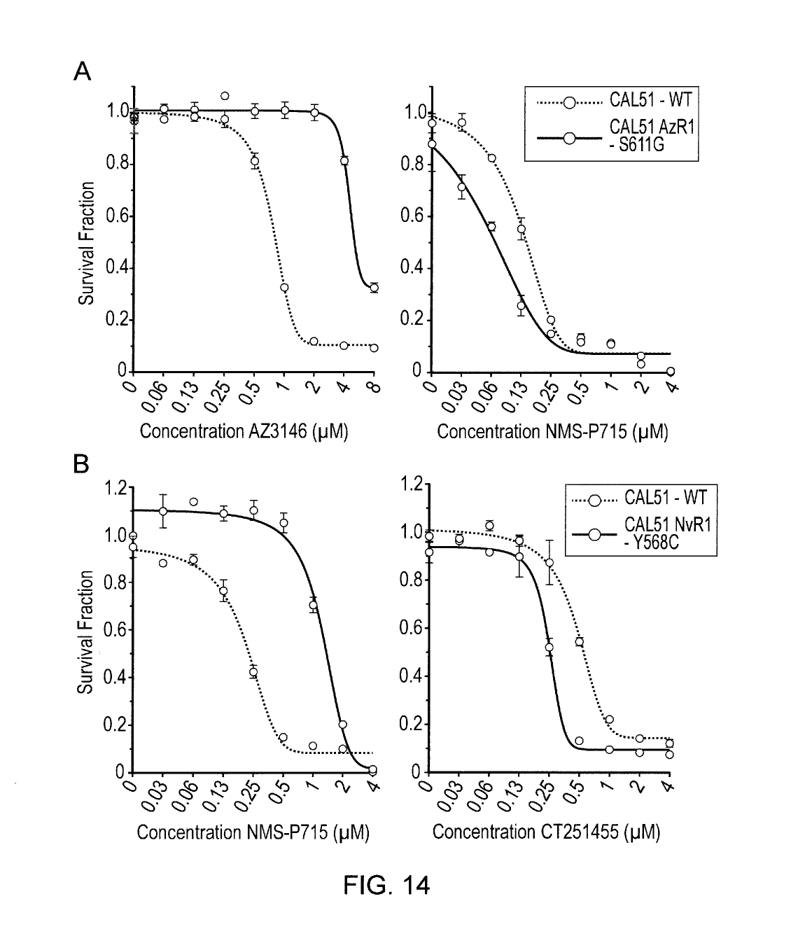

In order to investigate the mechanism through which human cancer cells could develop resistance against MPS1 inhibitors, we modified a previously established assay using HCT116 cells (32). HCT116 cells were cultured for 10 days in 0.8 μM (the GI₅₀) of the MPS1 inhibitor AZ3146 (7), then 2 μM AZ3146 for 3 weeks, a lethal concentration in cell viability assays (FIG. 1A). Under these conditions ˜60 colonies developed, from which 16 clones were isolated and cell lines generated, named AzR1-16. All 16 cell lines were resistant to AZ3146-induced cell death in cell viability assays (FIG. 1A-B, Table 1); AzR3 and 4 had a GI₅₀ of ˜3 μM (4-fold resistance), whilst the remaining 14 cell lines had a GI₅₀ of ˜9 μM (11-fold resistance, FIG. 1A). Since MPS1 is essential for the SAC, we reasoned that a likely cause of drug resistance would be MPS1 point mutations, thus we sequenced the cDNA of MPS1 from all resistant clones. Each cell line contained a single MPS1 point mutation (FIG. 1C); AzR3 and 4 contained a p.I531M mutation (c.1593A>G), whilst all the other clones possessed a p.S611G mutation (c.1831A>G, Table 1). Since all clones containing the same mutation had a similar fold-resistance to AZ3146, we selected one cell line containing each mutation to further characterize. To test if the MPS1-mutated cells still had a functional SAC in the presence of the inhibitor, we analysed their cell cycle profiles by flow cytometry. In the parental cell line, the G1 and G2/M peaks are increasingly abolished following treatment with 1 and 2 μM AZ3146 (FIG. 1D), whilst AzR1 and AzR3 were unaffected up to 4 and 2 μM AZ3146, consistent with a functional SAC. Likewise, when analysing mitosis by time-lapse microscopy (FIG. 1E), although treatment with 2 μM AZ3146 caused the parental cell line to rapidly exited mitosis in 10 min, AzR1 and 3 remained in mitosis for the normal length of time (˜25 mins), with no apparent mitotic defects, confirming a functional SAC.

TABLE 1
GI50 values and the mutations detected in each HCT116
AzR clone selected using AZ3146. Mutations were detected using
Sanger sequencing. The GI₅₀ values represent the mean of three
experiments

| Cell line | Mutation | GI₅₀ |
|---|---|---|
| name | detected | AZ3146 (μM) |
| AzR1 | p.S611G | 9.25 |
| AzR2 | p.S611G | 7.03 |
| AzR3 | p.I531M | 2.98 |
| AzR4 | p.I531M | 3.30 |
| AzR5 | p.S611G | 8.06 |
| AzR6 | p.S611G | 10.41 |
| AzR7 | p.S611G | 8.51 |
| AzR8 | p.S611G | 9.32 |
| AzR9 | p.S611G | 8.57 |
| AzR10 | p.S611G | 7.61 |
| AzR11 | p.S611G | 8.60 |
| AzR12 | p.S611G | 9.22 |
| AzR13 | p.S611G | 8.56 |
| AzR14 | p.S611G | 9.56 |
| AzR15 | p.S611G | 9.13 |
| AzR16 | p.S611G | 8.25 |

Having created cell lines resistant to AZ3146 (an 8-oxopurine), we subsequently investigated whether these cells are resistant to number of different structural classes of MPS1 inhibitors; a diaminopyridine (ONCOII), triaminopyridine (SNG12) and a pyrazoloquinazoline (NMS-P715), suggesting whether the mutations could cause cross-resistance in the clinic (FIG. 1F). Using a cell viability assay, we show that the p.I531M-containing AzR3 cell clone was also resistant to the OncoTherapy compound II (ONCOII, WO 2011/016472A1) and NMS-P715(6) although no resistance was seen to the Shionogi compound 12 (SNG12) (33). The p.S611G-containing AzR1 cells conferred up to 10 fold resistance against the ONCOII and SNG12 inhibitors, however showed no resistance against NMS-P715. These data suggest that the p.I531M and p.S611G mutations are able to confer resistance against a number of structurally different MPS1 inhibitors that bind to the hinge region of MPS1 kinase domain.

In order to confirm that the MPS1 mutations were sufficient to cause resistance to MPS1 inhibitors, we ectopically expressed the p.I531M the p.S611G and the double mutant (termed Db1) in DLD1 Flp-In T-Rex cells (FIG. 8A-B). Viability assays confirmed that while over-expression of wild-type MPS1 was unable to confer resistance to AZ3146, all three mutant constructs did confer resistance, with the Db1 construct being most effective.

Importantly, this drug-resistance was also associated with the rescue of SAC override (FIG. 8C-E). Expression of p.I531M, but not p.S611G, could also rescue cell survival following NMS-P715 treatment (FIG. 8F), confirming that the p.S611G mutation does not confer resistance to NMS-P715. Thus, these data confirm that expression of drug-resistant mutants alone confer resistance to MPS1 inhibitors whilst not adversely affect mitosis.

To determine the potential effect of the mutations on MPS1 kinase activity, we performed immunoprecipitation (IP)-kinase assays of

Myc-tagged MPS1 constructs. All three constructs phosphorylated themselves and myelin basic protein (MBP) to near WT levels, suggesting they have normal activity (FIG. 1H). In addition, we also measured the inhibition of MPS1 activity of the different constructs using an in-cell, fluorescence-based kinase assay using Meso Scale Discovery (MSD) electrochemiluminescence technology (19), quantifying MPS1 T33/S37 auto-phosphorylation as a marker for kinase activity. The wild-type construct had an IC₅₀ of 1.48 μM, the p.I531M 3.4 μM, the p.S611G 19.2 μM, whilst the double mutant had an IC₅₀>25 μM (FIG. 1I). This MPS1 phosphorylation was further confirmed by immunoblotting (FIG. 8G-H). We also generated recombinant full-length MPS1 p.S611G, which was 40-, and 15-fold more resistant to ONCOII and AZ3146 compared to wild-type recombinant protein (Table 2), whilst NMS-P715 was equipotent against the wild-type and p.S611G MPS1 proteins. These data confirm that the mutations prevent the inhibition of MPS1 by the small molecule inhibitor AZ3146.

TABLE 2
in vitro biochemical MPS1 inhibition. The ATP Kₘ values
for WT, S611G and C604W MPS1 protein were 10.7 ± 1.2 μM, 21 ± 8 μM
and 125 ± 7 μM, respectively. IC₅₀ values are expressed as mean ±
standard deviation from quadruplicate measurements.

| | IC₅₀ values at ATP Kₘ (nM) | | | IC₅₀ values at 1 mM ATP (nM) | | |
|---|---|---|---|---|---|---|
| Compound | WT | S611G | C604W | WT | S611G | C604W |
| AZ3146 | 5.5 ± 1.1 | 93 ± 6 | 530 ± 110 | 110 ± 29 | 1700 ± 580 | 1600 ± 280 |
| ONCOII | 10.8 ± 3.7 | 350 ± 98 | 2600 ± 650 | 110 ± 18 | 4400 ± 1600 | 8300 ± 2900 |
| NMS-P175 | 2.5 ± 0.9 | 3.0 ± 0.7 | 510 ± 130 | 8.8 ± 2.3 | 13.8 ± 5.5 | 1300 ± 120 |
| CCT251455 | 1.3 ± 0.4 | 7.8 ± 2.5 | 62 ± 36 | 4.0 ± 0.7 | 60 ± 22 | 140 ± 24 |
| Compound 1 | 3.8 ± 1.3 | 10.8 ± 3.1 | 80 ± 26 | 76 ± 20 | 160 ± 51 | 230 ± 48 |
| Compound 2 | 3.3 ± 1.1 | 22 ± 7 | 13.3 ± 2.9 | 60 ± 20 | 310 ± 77 | 16.3 ± 4.0 |

### The Generation of NMS-P715-Resistant Cell Lines

Having created cell lines resistant to AZ3146, we subsequently investigated whether different mutations would emerge using a structurally different chemical class. Therefore, we generated HCT116 clones resistant to NMS-P715, the only MPS1 inhibitor tested unaffected by the p.S611G mutation. Sequencing of the cDNA from 35 clones (NvR1-35) identified three new mutations: 5 clones contained a p.M600T (c.1799T>C), 9 clones contained a p.Y568C (c.1703A>G) and 20 clones contained a p.C604W (c.1812T>G) mutation (FIG. 2A, Table 3). One clone contained the previously characterized p.I531M mutation. All cell lines were resistant to NMS-P715-induced cell killing in cell viability assays (FIG. 2B-C, Table 3), conferring a similar fold resistance. However, this loss of cell viability was not accompanied by the typical loss of cell cycle profile associated with 24 hours MPS1 inhibition (FIG. 2D), suggesting NMS-P715 causes cell death in an additional off-target manner at 2 μM. To verify that the cell lines contained a function SAC, the mitosis of the HCT116 cell lines were analysed by time-lapse microscopy (FIG. 2E). Upon treatment with 1 μM NMS-P715, only the parental HCT116 cells rapidly exited mitosis, suggesting that the p.M600T, p.Y568C and p.C604W mutations prevent SAC override induced by NMS-P715.

TABLE 3
GI₅₀ values and the mutations detected in each HCT116 NvR
clone selected using NMS-P715. Mutations were detected using
Sanger sequencing. The GI₅₀ values represent the mean of three
experiments

| Cell line | Mutation | GI₅₀ |
|---|---|---|
| name | detected | NMS-P715 (mM) |
| NvR1 | p.M600T | 2.54 |
| NvR2 | p.M600T | 1.85 |
| NvR3 | p.I531M | 1.97 |
| NvR4 | p.M600T | 2.25 |
| NvR5 | p.M600T | 2.48 |
| NvR6 | p.M600T | 2.32 |
| NvR7 | p.Y568C | 3.55 |
| NvR8 | p.Y568C | 3.35 |
| NvR9 | p.C604W | 2.15 |
| NvR10 | p.Y568C | 3.11 |
| NvR11 | p.Y568C | 2.15 |
| NvR12 | p.C604W | 2.43 |
| NvR13 | p.C604W | 4.25 |
| NvR14 | p.C604W | 3.73 |
| NvR15 | p.C604W | 3.82 |
| NvR16 | p.C604W | 3.26 |
| NvR17 | p.C604W | 3.76 |
| NvR18 | p.C604W | 3.76 |
| NvR19 | p.C604W | 3.50 |
| NvR20 | p.C604W | 3.86 |
| NvR21 | p.C604W | 2.12 |
| NvR22 | p.C604W | 2.20 |
| NvR23 | p.C604W | 2.27 |
| NvR24 | p.C604W | 2.24 |
| NvR25 | p.C604W | 4.33 |
| NvR26 | p.C604W | 2.25 |
| NvR27 | p.Y568C | 3.55 |
| NvR28 | p.Y568C | 3.78 |
| NvR29 | p.C604W | 4.08 |
| NvR30 | p.C604W | 1.24 |
| NvR31 | p.Y568C | 3.64 |
| NvR32 | p.Y568C | 1.87 |
| NvR33 | p.Y568C | 1.96 |
| NvR34 | p.C604W | 2.09 |
| NvR35 | p.C604W | 2.44 |

Overexpression of p.M600T, p.Y568C or p.C604W mutant constructs in DLD1 Flp-In T-Rex cells (FIG. 9A-B), showed that each mutant was sufficient to confer resistance against NMS-P715 mediated cell death (FIG. 2F), restore the cell cycle profiles and prevent override of the SAC caused by NMS-P715 (FIG. 9C-E). IP-kinase assays showed that p.Y568C and p.C604W mutants phosphorylated themselves and MBP to wild-type levels, whereas the p.M600T mutant was only auto-phosphorylated to 20% of wild-type levels (FIG. 2G). Despite this, the p.M600T mutant still robustly phosphorylated MBP to 92% of wild-type levels and significantly, all Myc-MPS1 mutants were phosphorylated to the same extent in cells during mitosis, confirming that they are all comparably active (FIG. 9F). In MSD assays, all 3 mutants were resistant to NMS-P175, having an IC₅₀ of: 3.5 μM (4-fold), 8.2 μM (10-fold) and >16 μM (>21-fold) for the p.M600T, p.Y568C and p.C604W mutants, compared to ˜0.77 μM for the wild-type construct (FIG. 2H). Additionally, while p.I531M also conferred resistance in the MSD assay, p.S611G was ineffective (FIG. 2H). We also demonstrated that p.M600T, p.Y568C and p.C604W mutants were all able to confer resistance against AZ3146, ONCOII and SNG12 (FIG. 2I). Furthermore, a recombinant full-length MPS1 p.C604W mutant conferred 15-fold resistance to AZ3146, 75-fold resistance to ONCOII and 148-fold resistance to NMS-P715, compared to the wild-type protein (Table 2). These data together suggest, that NMS-P715 causes cell death through additional off-target effects

CCT251455: A Potent and Selective MPS1 Inhibitor that Overcomes Resistance Caused by the p.Y5680 Mutation

We have recently reported the discovery of a potent and selective MPS1 inhibitor CCT251455 (a pyrrolopyridine) with a GI₅₀ of 0.16 μM in HCT116 cells (FIG. 3A) (19). Using the MSD assay to measure MPS1 auto-phosphorylation at T676 and 133/S37, MPS1 kinase activity was inhibited at 0.22 and 0.04 μM respectively, consistent with the cell viability data (FIG. 3B). To confirm that CCT251455 induces phenotypes associated with MPS1 inhibition, we showed that the cell cycle profiles of HCT116 cells were abolished following 24 hour treatment with 0.32 μM CCT251455 (FIG. 3C). Likewise, HeLa cells rapidly exited mitosis upon CCT251455-treatmented in ˜17 min, 98% of which had unaligned chromosomes (FIG. 3D). CCT251455 was also able to abrogate a previously established taxol or nocodazole-induced SAC; within 1 hour following inhibitor treatment ˜100% of the cells had exited mitosis (FIG. 3E). CCT251455 also severely inhibited the kinetochore recruitment of MAD2, MAD1, ZW10 and CDC20, while BUB1 and BUBR1 were reduced, but still visible by microscopy (FIG. 3F and ig. 10), consistent with previous reports (7, 34). Conversely, MPS1 kinetochore localisation increased in the presence of CCT251455, although the pT33/S37 and pT676 signals were no longer visible, confirming that inactive MPS1 binds to the kinetochore (7, 35). As controls, the kinetochore localisation of ZWINT-1, CENP-E, CENP-F and CENP-A pS7 (a marker of Aurora B activity) remained unaffected by inhibitor-treatment (ig. 10), consistent with the inhibition of MPS1 kinase activity.

To show whether CCT251455-induced cell death is specifically through MPS1 inhibition, we tested CCT251455 in our five drug-resistant HCT116 cell lines (FIG. 3G). Both the p.S611G (AzR1) and p.I531M (AzR3) mutations conferred resistance, with p.S611G being most resistant at ˜10-fold. The p.C604W mutant cells (NvR12) were almost as resistant as p.S611G, while the p.M600T mutant (NvR1) conferred only 2-fold resistance. However, the p.Y568C mutation (NvR11) did not confer any resistance against CCT251455. This resistance was further confirmed using the MSD assay with the Db1 mutant showing most resistance at 3.9 μM (46-fold, FIG. 3H). Consistent with these data, CCT251455 was 15- and 35-fold less effective against the recombinant p.S611G and p.C604W MPS1 proteins compared to the wild-type, respectively (Table 2). Interestingly, when we generated drug-resistant HCT116 cell clones against CCT251455 using the same protocol as for AZ3146 and NMS-P715, we identified the p.S611G mutation in all clones (ig. 11). These data together demonstrate that CCT251455 kills cells specifically through MPS1 inhibition.

Crystal Structures of MPS1 p.S611G in Complex with MPS1 Inhibitors

To provide insight into the structural basis for the observed resistance of MPS1 to the inhibitors, we introduced the p.S611G mutation into an MPS1 kinase domain construct (MPS1-KD, residues 519-808) used for crystallisation experiments (19) and solved the crystal structures of the native (FIG. 12) and p.S611G MPS1-KD enzyme in binary complexes with the 8-oxopurine (AZ3146), the diaminopyridine (ONCOII) and the pyrrolopyridine compound 1, a close structural analogue of CCT251455.

To our surprise the binding of AZ3146 to the wild-type and p.S611G mutant MPS1-KD enzymes is almost identical (FIG. 4A). AZ3146 binds with two hydrogen bonds to the hinge, one between the purine N1 and Gly605NH atoms, the other between the anilino NH and Gly6050 atoms (FIG. 4B). The N7-methyl group of AZ3146 packs against the gatekeeper Met602 residue, and the N9-cyclopentyl group projects into the space occupied by the N-Boc substituent of CCT251455 bound to MPS1. The 2-methoxyanilino moiety projects towards solvent, positioning the piperidine group above the helix-capping Asp608-Ser611 motif (FIG. 4A). The activation loop was not resolved in either of these structures, most likely due to the use of PEG300 in the crystallization conditions, as noted previously (19).

The diaminopyridine (ONCOII) also bound in a very similar manner to the native and mutant enzymes in the crystal structures (FIG. 4B), comparable to the related diaminopyridine inhibitor reported in PDB entry 3VQU. In these crystal structures, the main chain peptide of the gatekeeper+2 residue, Cys604, is flipped relative to other inhibitor-bound MPS1KD structures, and provides the hinge-binding hydrogen bond interaction between the Cys604 carbonyl with the anilino NH of the inhibitor (FIG. 4B). The anilino substituent of ONCOII overlays well with the benzamide of the diaminopyridine inhibitor in 3VQU, but projects further towards Ser611, to a similar extent as the methylimidazole group of CCT251455. The 3-methoxynitrile aniline substituent occupies the selectivity pocket next to the side chains of Cys604 and Gln541 and above the post-hinge residues 605-607, also exploited by other MPS1 inhibitors (6, 19). The pyridine-5-cyano group that points towards the Lys553NZ atom does not appear to be a productive interaction. In the wild-type ONCOII-MPS1-KD structure we also observed the almost complete ordering of the activation loop, similar to that observed for a pyrimidodiazipine inhibitor (9), but this was less well-ordered for the p.S611G MPS1-KD bound to ONCOII with a short ordered segment (residues 671-674) contacting the cyclohexyl group of ONCOII, and residues 669-682 not visible in the electron density. However, this is most likely due to the use of PEG300 in crystallisation conditions (19). Likewise, the structure of p.S611G MPS1-KD in a complex with compound 1 (FIG. 4C) revealed a binding mode nearly identical to the previously reported WT MPS1-KD (PDB 4C4H) (19) and very similar to CCT251455.

In summary, the crystal structures of both WT and p.S611G MPS1-KD bound to three different classes of MPS1 inhibitors show only minor differences in inhibitor binding mode between the wild-type and p.S611G mutant proteins. However, importantly, in all three ligand-bound p.S611G MPS1-KD structures the p.S611G mutation was clearly apparent from the electron density surrounding this residue. Notably, this mutation removes the helix-capping interaction of the Ser side chain with Asp608, and the main chain of the resulting Gly residue is also more flexible; it is therefore likely that the S611G mutation results in greater flexibility of the αD helix. In support of this hypothesis, NMS-P715 is the only inhibitor we have tested with a potency not affected by the S611G mutation and which has a binding mode that is incompatible with the ordering of the activation loop. Therefore, we propose that the conformation of the activation loop residues, which may be affected by the p.S611G mutation, plays an important role in inhibitor resistance.

### p.I531M and p.C604W Mutations Obstruct MPS1 Inhibitor-Binding

Molecular modeling of the p.I531M mutation using the crystal structure of MPS1-KD in complex with ATP (PDB code 3HMN) shows that the p.I531M mutation would not be expected to abrogate MPS1 kinase activity, as a small subset of the commonly observed rotamers of the larger methionine side-chain can still be accommodated next to the bound nucleotide (FIG. 5A). However, modeling the p.I531M mutation in the MPS1-KD structure bound to AZ3146 shows that all methionine rotamers would clash with the anilino moiety or the cyclopentyl group of the inhibitor (FIG. 5B). Therefore, it is likely that the I531M mutation would confer resistance to any inhibitor containing both a large group equivalent to the anilino moiety and a substituent similar to the cyclopentyl group of AZ3146. This hypothesis is supported by the fact that the only inhibitor against which the p.I531M mutation in cells did not confer resistance is the recently reported triaminopyridine inhibitor (SNG12, FIG. 1F), which contains a smaller aniline substituent compared to the other tested MPS1 inhibitors.

In addition to the p.S611G MPS1-KD, we were also able to elucidate the crystal structures of the p.C604W MPS1-KD mutant in complex with the pyrazoloquinazoline NMS-P715. The p.C604W mutation was clearly observed in the electron density after molecular replacement, indicating that the Trp side chain is well-ordered in this structure. In comparison with the crystal structure of wild-type MPS1 bound to NMS-P715 (PDB code 2X9E), the carbonyl group of Gly605 is rotated towards the ligand, due to steric hindrance by the bulky Trp604 side-chain of the mutant protein (FIG. 5C). In both the wild-type and p.C604W mutant structures, the ligand makes two H-bonds to the hinge and an additional H-bond between Lys553 and an amide oxygen atom. The latter H-bond acts as the anchor point for rotation of the ligand away from the hinge region in the C604W MPS1 mutant compared to the wild-type enzyme. This rotation is caused by the bulky Trp604 side chain, which would otherwise sterically clash with the trifluoromethoxy group of the ligand. All of the other inhibitors used in this study contain a substitution comparable to the anilino 2-trifluoromethoxy group of NMS-P715, explaining the resistance conferred by the p.C604W mutation to all of the MPS1 inhibitors described so far. For ONCOII, the Trp mutation has a dual effect through causing both steric hindrance, as well as by loosing a hinge binding interaction with the anilino NH of the inhibitor.

### Discovery of MPS1 Inhibitors Overcoming MPS1 Resistant Mutants

Having shown that the p.C604W mutation caused resistance to all of the MPS1 inhibitors tested, we set out to design a compound to specifically overcome this mutation. Thus, we synthesised two related pyrrolopyridines in which the chlorine atom was replaced with hydrogen or fluorine (compound 2 and 3, FIG. 6A). When these compounds were tested in the drug-resistant cell lines, both were more potent towards NvR12 than the parental HCT116 cells, particularly compound 2, which was 5-fold more effective (FIG. 6B). However, the other drug resistant mutations still conferred resistance to the inhibitors, with p.S611G giving over 5-fold resistance, whilst the resistance conferred by p.Y568C, p.M600T and p.I531M were limited to ˜2-fold (FIG. 6B). We further confirmed this increased effectiveness against p.C604W by flow cytometry; compound 2 caused a loss of cell cycle profiles at 100 nM for NvR12, compared to 600 nM for the parental and >900 nM for AzR1 cells (FIG. 6C). Furthermore, in vitro, compound 2 was also approximately 5-fold more active against the recombinant p.C604W mutant compared to the wild-type protein, consistent with the cellular results (Table 2).

To understand why compound 2 was more active against the p.C604W mutation, we determined the structures of both wild-type and p.C604W MPS1-KD proteins with compound 2 (FIG. 5D-E). The binding of compound 2 is almost identical to CCT251455, with only a small difference in the torsion angles between aniline and imidazole rings of approximately 14°, due to the smaller H atom in the 2-position of the aniline of compound 2. In the compound 2-bound p.C604W mutant structure, the P-loop residues from Ser526 to Ser537 are located further away from the kinase hinge than in the wild-type structure due to the larger Trp604 side chain. This results in a larger hydrophobic surface defined by the side chains of Trp604 and Ile531 against which the aromatic anilino substituent packs. Furthermore, as observed in the p.C604W structure with NMS-P715, due to the larger side chain of Trp604, the carbonyl group of Gly605 is rotated towards the anilino NH of the inhibitor. An important difference between the structures of MPS1 with NMS-P715 and compound 2 is the lack of a 2-anilino substituent in the latter, meaning that compound 2 is not rotated away from the hinge by steric hindrance of the Trp604 side chain. Indeed, the H-bond distance between the anilino NH and the carbonyl oxygen atom of Gly605 is less than 2.8 Å in the p.C604W mutant structure compared with the equivalent distance of more than 3.2 Å in the wild-type structure. Therefore, the greater potency of compound 2 versus p.C604W mutant MPS1 relative to wild-type is most likely due to a combination of improved hinge-binding and more optimal hydrophobic interactions.

### Drug-Resistant Mutations are Pre-Existing in HCT116 Cells

Having discovered a number of mutations that confer drug-resistance against multiple MPS1 inhibitors, we aimed to determine whether these mutations are pre-existing within the cancer cell population. To this end, we optimized Small Nucleotide Polymorphism (SNP) assays using the QX100 Droplet Digital PCR System (ddPCR, Biorad) and Taqman primer-probes. An emulsion was made containing 10,000 gDNA-containing droplets, then following a PCR reaction, the fluorescence of each individual droplet was determined, allowing quantification of the wild-type and mutant alleles. The gate for each population was set according to controls, minimizing any false positives (determined using wild-type vector DNA spiked into Drosophila gDNA). In each drug-resistant HCT116 cell line, 43-50% of the droplets were positive for the corresponding mutant allele, confirming each cell line was heterozygous for the mutation (FIG. 7A-B). In parental HCT116 gDNA, the p.S611G mutation was the most frequent mutation at 0.94%, followed by p.Y568C at 0.3%, whilst the p.I531M and p.C604W mutations were the least frequent at 0.02% and 0.07% (FIG. 7C). The number of mutant-positive droplets increased proportionately with increasing concentrations of gDNA and decreased with dilution, suggesting that these mutant-positive droplets are a “true” signal (FIG. 13A-B). Furthermore, the FA of all mutants, except p.Y568C, increased between 10-50 fold in HCT116 cells after 3 days selection with 0.8 μM AZ3146 (FIG. 13C).

To address how specific these mutations were, we designed primer-probes for p.S611C and p.S611R mutations (A>T and A>C mutations, respectively), and Tyr568 mutated to a stop codon. When we tested HCT116 cells for these alternative mutations we did not detect a single droplet positive for any of the mutations (FIG. 7D), whilst 16-37.4% of the droplets were mutant positive when the gDNA was spiked with mutant vector. This confirms that Ser611 is only found mutated to a glycine residue, whilst Tyr568 is not mutated to a stop codon. Taken together, these data suggest that the drug-resistant mutations are specific and pre-existing within the HCT116 population and not due errors in the technique. In fact, we have seen that within 24 days outgrowth from a single cell, the drug-resistant mutations are detected in HCT116 cells (FIG. 13D). Conversely, using the p.S611G-containing AzR1 cells, the Gly611 allele is mutated back to Ser in up to 30% of the cells (FIG. 13E), suggesting these bases may be frequently mutated.

### Drug-Resistant Mutations can be Detected in Both Cancer Cells and Non-Transformed Cells

Since HCT116 cells contain a mismatch repair defect, we hypothesized that the FA of the mutations may be higher in this cell line compared to other cancer cells, thus we analysed a panel of 17 breast and pancreatic cancer cell lines. However, the drug-resistant mutations were typically identified in every cell line at strikingly similar levels (FIG. 7E and Table 4), suggesting that these mutations are present in all cancer-cell lines at similar frequencies. In agreement with this data, when we created drug-resistant cell lines against AZ3146 and NMS-P715 in CAL51 cells, the two mutations identified were p.S611G for AZ3146-treated cells and p.Y568C for NMS-P715-treated cells (FIG. 14).

TABLE 4
Fractional abundance of MPS1 mutants in breast and
pancreatic cell lines. Values equal to or below the false-
positive rate (calculated using digested MYC-MPS1 vector DNA
spiked into drosophila DNA) are reported as 0.

| | S611G | I531M | M600T | Y568C | C604W |
|---|---|---|---|---|---|
| SUM149PT | 0.679 | 0.053 | 0.358 | 0.414 | 0.370 |
| MDAMB231 | 0.443 | 0.041 | 0.148 | 0.380 | 0.168 |
| MDAMB468 | 0.421 | 0.062 | 0.175 | 0.196 | 0.180 |
| MDAMB453 | 0.278 | 0.043 | 0.174 | 0.409 | 0.095 |
| CAMA1 | 0.478 | 0.121 | 0.263 | 0.314 | 0.241 |
| MDAMB361 | 0.395 | 0 | 0.102 | 0.310 | 0.095 |
| BT549 | 0.308 | 0.233 | 0.262 | 0.156 | 0.133 |
| SUM52 | 0.976 | 0.080 | 0.264 | 0.737 | 0.147 |
| MFM223 | 0.799 | 0.118 | 0.340 | 0.642 | 0.154 |
| CAL51 | 0.787 | 0.044 | 0.255 | 0.702 | 0.243 |
| T47D | 0.865 | 0.123 | 0.377 | 0.780 | 0.243 |
| MCF7 | 0.895 | 0.028 | 0.247 | 0.675 | 0.135 |
| CAPAN-1 | 0.281 | 0.015 | 0.222 | 0.167 | 0.163 |
| BXPC3 | 0.401 | 0.026 | 0.212 | 0.281 | 0.082 |
| CFPCA-1 | 0.648 | 0.044 | 0.181 | 0.393 | 0.123 |
| PANC-1 | 0.281 | 0.039 | 0.124 | 0.125 | 0.078 |
| SUIT2 | 0.294 | 0.045 | 0.116 | 0.023 | 0.041 |

Next we investigated whether these mutations are found pre-existing in patient tumour samples, suggesting they could be selected for in the clinic. We analysed the gDNA of 14 treatment-naïve (BamHI-digested), invasive breast carcinomas of no special type (Table 5) (36). The p.S611G and p.Y568C mutations were detected in every tumour samples, although typically at a lower FA than in the cell lines; <0.2% and <0.09%, respectively (FIG. 7E). The p.I531M mutation was detected in 13 tumours at 0.1-0.4%, the p.C604W in 10 tumours at 0.03-0.37%, while the p.M600T was only detected in 5 samples. The mutations could also be detected in undigested tumour gDNA (Table 6), although in fewer samples and at a lower FA, likely due to a reduced efficiency in the PCR amplification.

TABLE 5
Breast cancer sample subtypes.

| | GRADE | ER | PR | HER2 | SUBTYPE |
|---|---|---|---|---|---|
| B1 | 3 | + | − | − | Luminal |
| B3 | 3 | + | + | − | Luminal |
| B4 | 2 | + | − | + | HER2 |
| B10 | 3 | − | − | + | HER2 |
| B12 | 3 | − | − | + | HER2 |
| B18 | 3 | − | − | − | Basal |
| B19 | ND | ND | ND | ND | ND |
| B20 | 3 | − | − | + | HER2 |
| BC184 | 3 | − | − | − | Basal |
| BC185 | 2 | + | − | + | HER2 |
| BC1921 | 3 | + | + | − | Luminal |
| BC2050 | 2 | + | + | − | Luminal |
| BC2067 | 2 | + | + | − | Luminal |
| BC2072 | 1 | + | + | − | Luminal |
| BC2241 | 1 | + | + | − | Luminal |
| BC2973 | 2 | + | + | − | Luminal |
| BC2974 | 1 | + | + | − | Luminal |
| BC2980 | 2 | + | + | − | Luminal |
| BC3014 | 2 | + | + | − | Luminal |
| BC3015 | 2 | + | + | − | Luminal |
| BC3017 | 2 | + | + | − | Luminal |
| BC3045 | 2 | − | + | − | Basal |
| BC3046 | 2 | + | − | − | Luminal |
| BC3048 | 2 | + | + | − | Luminal |
| BC3049 | ND | ND | ND | ND | ND |

ND = not determined

TABLE 6
Fractional abundance of MPS1 mutants in undigested
breast cancer samples. ND = not determined due to limited DNA.
Signals equal to or below the false positive rate (calculated
using digested MYC-MPS1 vector DNA spiked into drosophila DNA)
are reported as 0.

| | S611G | I531M | M600T | Y568C | C604W |
|---|---|---|---|---|---|
| B1 | 0 | 0 | 0 | 0 | 0.052 |
| B3 | 0.083 | 0 | 0 | 0 | 0.176 |
| B4 | 0.162 | 0 | 0.032 | 0 | 0.018 |
| B10 | 0.042 | 0 | 0 | 0 | 0.107 |
| B12 | 0.083 | 0 | 0 | 0 | 0.044 |
| B18 | 0 | 0 | 0 | 0 | 0.025 |
| B19 | 0.098 | 0 | 0.036 | 0 | 0.132 |
| B20 | 0.041 | 0 | 0 | 0.017 | 0.163 |
| BC184 | 0.217 | 0 | 0.066 | 0.096 | 0.218 |
| BC185 | 0.135 | 0 | 0.131 | 0.080 | 0.035 |
| BC1921 | 0 | 0 | 0.033 | 0.029 | 0.014 |
| BC 2050 | 0 | 0 | 0 | 0.022 | 0.018 |
| BC 2067 | 0 | 0.017 | 0 | 0 | 0.008 |
| BC 2072 | 0 | 0 | 0.103 | 0.208 | 0.015 |
| BC 2241 | 0.058 | 0 | 0.068 | 0 | 0 |
| BC 2973 | 0.235 | 0 | 0.024 | 0 | 0 |
| BC 2974 | 0.085 | ND | ND | 0.029 | 0.021 |
| BC 2980 | 0 | 0.016 | 0 | 0.027 | 0.012 |
| BC 3014 | 0.206 | 0.011 | 0.125 | 0.194 | 0.125 |
| BC 3015 | 0.128 | 0 | 0 | 0.032 | 0.040 |
| BC 3045 | 0.025 | 0 | 0.025 | 0 | 0 |
| BC 3046 | 0.085 | 0 | 0 | 0 | 0.008 |
| BC 3048 | 0.166 | 0 | 0.031 | 0.057 | 0.033 |
| BC 3049 | 0.043 | 0.017 | 0.026 | 0.050 | 0.056 |

In order to determine whether these pre-existing mutations were specific to cancer cells, we then also analysed 8 lymphoblast gDNA samples from healthy individuals. Surprisingly, each mutation was also identified in the majority of lymphoblast samples tested (FIG. 7F), again with the p.S611G mutation typically at the highest FA. We then also analysed 5 normal breast tissue samples for the presence of the p.S611G mutation. p.S611G was identified with a FA of 0.04-0.07% in 4 of the 5 samples (FIG. 12E), again suggesting that this mutation is pre-existing in normal, non-transformed cells.

Finally, to address whether mutations in other genes that confer acquired drug-resistance are also naturally occurring, we optimised a SNP assay to identify the EGFR p.T790M gatekeeper mutation, a major cause of resistance to gefitinib treatment. When we analysed the HCT116 gDNA alone, the p.T790M mutation was detected at a FA of 0.07%, which increased to 99.48% of droplets when spiked with synthetic Ultramer oligos (FIG. 7G). Furthermore, the p.T790M mutant was detected 12 of the 17 breast and pancreatic cancer cell lines tested, at <0.08% (Table 7A), in half the lymphoblast samples (Table 7B), as well as in 4 of normal breast tissue samples (FIG. 13F). These results would suggest that point mutations conferring acquired drug-resistance to MPS1 and EGFR inhibitors are naturally occurring mutations, pre-existing in both normal and cancer cells.

TABLE 7A
Fractional abundance of EGFR p.T790M mutant in breast
and pancreatic cell lines

| Cell lines | T790M |
|---|---|
| SUM149PT | 0.071 |
| MDAMB231 | 0.059 |
| MDAMB468 | 0.046 |
| MDAMB453 | 0.050 |
| CAMA1 | 0.057 |
| MDAMB361 | 0.050 |
| BT549 | 0.037 |
| SUM52 | 0.052 |
| MFM223 | 0 |
| CAL51 | 0 |
| T47D | 0.022 |
| MCF7 | 0 |
| CAPAN-1 | 0.041 |
| BXPC3 | 0 |
| CFPCA-1 | 0.077 |
| PANC-1 | 0.023 |
| SUIT2 | 0 |

TABLE 7B
Fractional abundance of EGFR p.T790M mutant in
lymphoblast samples

| Lymphoblast | EGFR |
|---|---|
| samples | T790M |
| 7 | 0 |
| 11 | 0.057 |
| 13 | 0.031 |
| 14 | 0 |
| 15 | 0.037 |
| 16 | 0.061 |
| 17 | 0 |
| 22 | 0 |

### Discussion

Whilst kinase inhibitors can be very effective in the clinic (37), their success has been limited by the emergence of drug-resistance. The most common and well-documented causes of drug resistance are mutations or amplifications of the drug target itself, or in alternative genes that activate parallel or downstream signaling pathways (20, 21). Here we describe the development of drug resistant HCT116 cell lines, using the MPS1 inhibitors AZ3146, NMS-P715 and CCT251455. Cell culture models have been used previously to successfully identify mechanisms of resistance that also develop in the clinic (38, 39). Each inhibitor resulted in the generation of common and drug-specific MPS1 point mutations, with each mutation conferring resistance against multiple MPS1 inhibitors, the effectiveness depending on the binding mode of the inhibitor. Although we identified 5 mutations contained within the ATP-binding pocket of MPS1, this neither excludes the possibility that other resistant mechanisms may exist, such as drug efflux pumps, nor that additional MPS1 mutations may also cause resistance. For example, BCR-ABL tolerates mutations in over 60 amino acid positions that confer drug resistance (24). Furthermore, ectopic expression of an MPS1 gatekeeper mutant (p.M602Q), can confer resistance to alternative MPS1 inhibitors (9).

In this paper we extensively characterize novel MPS1 mutations, both in function and their frequency in the population, presenting compelling evidence to explain why specific mutations consistently arise following inhibitor treatment. Using ddPCR, we show that each point mutation is pre-existing within every cancer cell line examined at similar frequencies, regardless of their mutational background. Crucially, when we looked for three alternative point mutations, none were detected in the cell population, suggesting that these mutations either do not occur, thus are specific in nature, or they may render MPS1 non-functional, thus are eliminated from the population. We also found that multiple inhibitors led to the selection of the most frequent and resistant p.S611G mutation, suggesting that important factors pertinent to the selection of a particular mutation including: 1) the fold-resistance the mutation confers, as well as 2) its FA in the population. Interestingly, when looking at the Cosmic or cBioPortal databases, a large number of mutations have been reported in the MPS1 gene in tumour samples, including p.G534E, p.D566G, Y599C, p.M600I, p.V601I and p.C604F; residues very close to, or the same as we found mutated in this study (p.I531M, p.Y568C, p.M600T, p.C604W and p.S611G). Whilst all these previously identified mutations are completely uncharacterized with no functional data reported, and in some cases are unverified, together with our data it suggests that the kinase domain of MPS1 may be frequently mutated in cancer cells, thus providing the potential for cells to develop acquired resistance against inhibitors. However, how frequently these other mutations are found in tumours or are pre-existing in normal tissue, whether they affect the function of MPS1, or whether they could confer resistance to MPS1 inhibitors is unknown. Pre-existing mutations being specific in nature would also explain why, despite the introduction of gatekeeper mutations into the BRAF^{V600E} protein conferring drug-resistance in vitro (40), these mutations have never been identified in cell lines or tissue samples. Thus, we speculate that these mutations are not naturally occurring in BRAF, or are much less frequent compared to other resistant mechanisms (41, 42).

A critical question for anti-cancer therapy is what is the origin/cause of acquired resistance. Our data indicate that acquired drug-resistance occurs through the selection of pre-existing genetic differences within the tumour population. Indeed, we show that these mutations are rapidly selected for in cells upon inhibitor treatment; increasing up to 50 fold in only 3-days selection with a GI₅₀ concentration. Mutations conferring resistance to BCR-ABL inhibitors have also been shown to be present in both pre and post-inhibitor treated tumours (24, 43). Likewise, the p.T790M gatekeeper mutation in EGFR, has been detected pre-treatment in non-small cell lung cancer, although this mutation is thought to have some oncogenic properties (44-46). Our data significantly expands upon these previous studies in showing, for the first time, that both MPS1 and EGFR drug-resistant mutations are pre-existing not only in a large number of cancer cell lines and tumours, but are also naturally occurring in healthy, normal lymphoblast and breast tissues. This result is contrary to pre-existing MET amplifications (causing resistance to gefitinib), which is suggested to be cell-line specific (47). This suggests that the origin of mutations causing acquired resistance may not be a result of high mutagenic rates in cancer cells as previously thought, but from naturally occurring mutations in normal tissues. Whilst we cannot rule out some low-level selective advantage for these MPS1 mutations, we believe that their constant low levels in cancer cell lines, as well as their emergence within weeks of expanding clonal populations, suggests that these residues are frequently and specifically mutated.

The knowledge that mutations conferring resistance to kinase-inhibitor therapy are pre-existing in normal cells highlights the need to identify strategies to overcome drug-resistance early during drug development. Whilst the p.S611G mutation typically caused high resistance to all inhibitors tested, NMS-P715 was unaffected, highlighting the potential to synthesize compounds to overcome this common resistant mutation. Likewise, the p.I531M and p.Y568C mutations were not effective at causing resistance to SNG12 and 001251455, respectively. However, of concern, we found that the p.C604W MPS1 mutation conferred resistance to all the inhibitors tested, due to the steric hindrance caused by the bulky Trp residue in the hinge binding region. Nevertheless, based on the crystal structure of CCT251455 bound to MPS1, we were able to design 2 compounds that not only avoid this clash, but which more potently targeted the mutant compared to wild-type kinase. Since all the mutations identified in this study were pre-existing in cancer cells, it would suggest that the development of acquired resistance is an inevitable outcome following inhibitor treatment with a single agent. However, since different inhibitors remain effective against distinct mutations, we would suggest that using a variety of MPS1 inhibitors, either in combination or via cyclical treatment, may be beneficial in combating the development of resistance. Alternatively, by monitoring the development of mutations in a relapsing tumour, it would be possible to then select the appropriate inhibitor to overcome the resistance as a second line treatment.

In conclusion, our data would agree with Diaz and colleagues that resistance is a “fait accompli” (48). However, we demonstrate that the drug-resistant mutations are actually pre-existing in normal, as well as cancer cells, most likely being introduced during continued proliferation. This would explain why acquired resistance is so rapidly encountered in the clinic with targeted therapies and suggests it is imperative to identify and prepare strategies to address this issue early during drug discovery.

### REFERENCES

The documents disclosed herein are all expressly incorporated by reference in their entirety.
1. Lara-Gonzalez P, Westhorpe F G, Taylor S S. The spindle assembly checkpoint. Current biology: CB. 2012; 22(22):R966-80.
2. Lauze E, Stoelcker B, Luca F C, Weiss E, Schutz A R, Winey M. Yeast spindle pole body duplication gene MPS1 encodes an essential dual specificity protein kinase. The EMBO journal. 1995; 14(8):1655-63.
3. Abrieu A, Magnaghi-Jaulin L, Kahana J A, Peter M, Castro A, Vigneron S, et al. Mps1 is a kinetochore-associated kinase essential for the vertebrate mitotic checkpoint. Cell. 2001; 106(1):83-93.
4. Maure J F, Kitamura E, Tanaka TU. Mps1 kinase promotes sister-kinetochore bi-orientation by a tension-dependent mechanism. Current biology: CB. 2007; 17(24):2175-82.
5. Tighe A, Staples O, Taylor S. Mps1 kinase activity restrains anaphase during an unperturbed mitosis and targets Mad2 to kinetochores. The Journal of cell biology. 2008; 181(6):893-901.
6. Colombo R, Caldarelli M, Mennecozzi M, Giorgini M L, Sola F, Cappella P, et al. Targeting the mitotic checkpoint for cancer therapy with NMS-P715, an inhibitor of MPS1 kinase. Cancer research. 2010; 70(24):10255-64.
7. Hewitt L, Tighe A, Santaguida S, White A M, Jones C D, Musacchio A, et al. Sustained Mps1 activity is required in mitosis to recruit 0-Mad2 to the Mad1-C-Mad2 core complex. The Journal of cell biology. 2010; 190(1):25-34.
8. Jemaa M, Galluzzi L, Kepp O, Senovilla L, Brands M, Boemer U, et al. Characterization of novel MPS1 inhibitors with preclinical anticancer activity. Cell death and differentiation. 2013; 20(11):1532-45.
9. Kwiatkowski N, Jelluma N, Filippakopoulos P, Soundararajan M, Manak M S, Kwon M, et al. Small-molecule kinase inhibitors provide insight into Mps1 cell cycle function. Nature chemical biology. 2010; 6(5):359-68.
10. Tardif K D, Rogers A, Cassiano J, Roth B L, Cimbora D M, McKinnon R, et al. Characterization of the cellular and antitumor effects of MPI-0479605, a small-molecule inhibitor of the mitotic kinase Mps1. Molecular cancer therapeutics. 2011; 10(12):2267-75.
11. Daniel J, Coulter J, Woo J H, Wilsbach K, Gabrielson E. High levels of the Mps1 checkpoint protein are protective of aneuploidy in breast cancer cells. Proceedings of the National Academy of Sciences of the United States of America. 2011; 108(13):5384-9.
12. Landi M T, Dracheva T, Rotunno M, Figueroa J D, Liu H, Dasgupta A, et al. Gene expression signature of cigarette smoking and its role in lung adenocarcinoma development and survival. PloS one. 2008; 3(2):e1651.
13. Salvatore G, Nappi T C, Salerno P, Jiang Y, Garbi C, Ugolini C, et al. A cell proliferation and chromosomal instability signature in anaplastic thyroid carcinoma. Cancer research. 2007; 67(21):10148-58.
14. Tannous B A, Kerami M, Van der Stoop P M, Kwiatkowski N, Wang J, Zhou W, et al. Effects of the selective MPS1 inhibitor MPS1-IN-3 on glioblastoma sensitivity to antimitotic drugs. Journal of the National Cancer Institute. 2013; 105(17):1322-31.
15. Thykjaer T, Workman C, Kruhoffer M, Demtroder K, Wolf H, Andersen L D, et al. Identification of gene expression patterns in superficial and invasive human bladder cancer. Cancer research. 2001; 61(6):2492-9.
16. Yuan B, Xu Y, Woo J H, Wang Y, Bae Y K, Yoon D S, et al. Increased expression of mitotic checkpoint genes in breast cancer cells with chromosomal instability. Clinical cancer research: an official journal of the American Association for Cancer Research. 2006; 12(2):405-10.
17. Slee R B, Grimes B R, Bansal R, Gore J, Blackburn C, Brown L, et al. Selective Inhibition of Pancreatic Ductal Adenocarcinoma Cell Growth by the Mitotic MPS1 Kinase Inhibitor NMS-P715. Molecular cancer therapeutics. 2014; 13(2):307-15.
18. Brough R, Frankum J R, Sims D, Mackay A, Mendes-Pereira A M, Bajrami I, et al. Functional viability profiles of breast cancer. Cancer discovery. 2011; 1(3):260-73.
19. Naud S, Westwood I M, Faisal A, Sheldrake P, Bavetsias V, Atrash B, et al. Structure-based design of orally bioavailable 1H-pyrrolo[3,2-c]pyridine inhibitors of mitotic kinase monopolar spindle 1 (MPS1). Journal of medicinal chemistry. 2013; 56(24):10045-65.
20. Niederst M J, Engelman J A. Bypass mechanisms of resistance to receptor tyrosine kinase inhibition in lung cancer. Science signaling. 2013; 6(294):re6.
21. Workman P, Al-Lazikani B, Clarke P A. Genome-based cancer therapeutics: targets, kinase drug resistance and future strategies for precision oncology. Current opinion in pharmacology. 2013; 13(4):486-96.
22. Apperley J F. Part I: mechanisms of resistance to imatinib in chronic myeloid leukaemia. The lancet oncology. 2007; 8(11):1018-29.
23. Shah N P, Nicoll J M, Nagar B, Gorre M E, Paquette R L, Kuriyan J, et al. Multiple BCR-ABL kinase domain mutations confer polyclonal resistance to the tyrosine kinase inhibitor imatinib (STI571) in chronic phase and blast crisis chronic myeloid leukemia. Cancer cell. 2002; 2(2):117-25.
24. Soverini S, Martinelli G, Rosti G, Iacobucci I, Baccarani M. Advances in treatment of chronic myeloid leukemia with tyrosine kinase inhibitors: the evolving role of Bcr-Abl mutations and mutational analysis. Pharmacogenomics. 2012; 13(11):1271-84.
25. Rosti G, Castagnetti F, Gugliotta G, Palandri F, Baccarani M. Second-generation BCR-ABL inhibitors for frontline treatment of chronic myeloid leukemia in chronic phase. Critical reviews in oncology/hematology. 2012; 82(2):159-70.
26. Hughes T, Saglio G, Branford S, Soverini S, Kim D W, Muller M C, et al. Impact of baseline BCR-ABL mutations on response to nilotinib in patients with chronic myeloid leukemia in chronic phase. Journal of clinical oncology: official journal of the American Society of Clinical Oncology. 2009; 27(25):4204-10.
27. Muller M C, Cortes J E, Kim D W, Druker B J, Erben P, Pasquini R, et al. Dasatinib treatment of chronic-phase chronic myeloid leukemia: analysis of responses according to preexisting BCR-ABL mutations. Blood. 2009; 114(24):4944-53.
28. Chan W W, Wise S C, Kaufman M D, Ahn Y M, Ensinger C L, Haack T, et al. Conformational control inhibition of the BCR-ABL1 tyrosine kinase, including the gatekeeper T315I mutant, by the switch-control inhibitor DCC-2036. Cancer cell. 2011; 19(4):556-68.
29. Zhang J, Adrian F J, Jahnke W, Cowan-Jacob S W, Li A G, Iacob R E, et al. Targeting Bcr-Abl by combining allosteric with ATP-binding-site inhibitors. Nature. 2010; 463(7280):501-6.
30. Cortes J E, Kim D W, Pinilla-Ibarz J, le Coutre P, Paquette R, Chuah C, et al. A phase 2 trial of ponatinib in Philadelphia chromosome-positive leukemias. The New England journal of medicine. 2013; 369(19):1783-96.
31. O'Hare T, Shakespeare W C, Zhu X, Fide C A, Rivera V M, Wang F, et al. AP24534, a pan-BCR-ABL inhibitor for chronic myeloid leukemia, potently inhibits the T315I mutant and overcomes mutation-based resistance. Cancer cell. 2009; 16(5):401-12.
32. Girdler F, Sessa F, Patercoli S, Villa F, Musacchio A, Taylor S. Molecular basis of drug resistance in aurora kinases. Chemistry & biology. 2008; 15(6):552-62.
33. Kusakabe K-i, Ide N, Daigo Y, Itoh T, Higashino K, Okano Y, et al. Diaminopyridine-Based Potent and Selective Mps1 Kinase Inhibitors Binding to an Unusual Flipped-Peptide Conformation. ACS Medicinal Chemistry Letters. 2012; 3(7):560-4.
34. Sliedrecht T, Zhang C, Shokat K M, Kops G J. Chemical genetic inhibition of Mps1 in stable human cell lines reveals novel aspects of Mps1 function in mitosis. PloS one. 2010; 5(4):e10251.
35. Jelluma N, Dansen T B, Sliedrecht T, Kwiatkowski N P, Kops G J. Release of Mps1 from kinetochores is crucial for timely anaphase onset. The Journal of cell biology. 2010; 191(2):281-90.
36. Buerger H, Otterbach F, Simon R, Schafer K L, Poremba C, Diallo R, et al. Different genetic pathways in the evolution of invasive breast cancer are associated with distinct morphological subtypes. The Journal of pathology. 1999; 189(4):521-6.
37. Druker B J, Guilhot F, O'Brien S G, Gathmann I, Kantarjian H, Gattermann N, et al. Five-year follow-up of patients receiving imatinib for chronic myeloid leukemia. The New England journal of medicine. 2006; 355(23):2408-17.
38. Engelman J A, Zejnullahu K, Mitsudomi T, Song Y, Hyland C, Park J O, et al. MET amplification leads to gefitinib resistance in lung cancer by activating ERBB3 signaling. Science. 2007; 316(5827):1039-43.
39. Moore A S, Faisal A, Gonzalez de Castro D, Bavetsias V, Sun C, Atrash B, et al. Selective FLT3 inhibition of FLT3-ITD+acute myeloid leukaemia resulting in secondary D835Y mutation: a model for emerging clinical resistance patterns. Leukemia. 2012; 26(7):1462-70.
40. Whittaker S, Kirk R, Hayward R, Zambon A, Viros A, Cantarino N, et al. Gatekeeper mutations mediate resistance to BRAF-targeted therapies. Science translational medicine. 2010; 2(35):35ra41.
41. Emery C M, Vijayendran K G, Zipser M C, Sawyer A M, Niu L, Kim J J, et al. MEK1 mutations confer resistance to MEK and B-RAF inhibition. Proceedings of the National Academy of Sciences of the United States of America. 2009; 106(48):20411-6.
42. Montagut C, Sharma S V, Shioda T, McDermott U, Ulman M, Ulkus L E, et al. Elevated CRAF as a potential mechanism of acquired resistance to BRAF inhibition in melanoma. Cancer research. 2008; 68(12):4853-61.
43. Pfeifer H, Lange T, Wystub S, Wassmann B, Maier J, Binckebanck A, et al. Prevalence and dynamics of bcr-abl kinase domain mutations during imatinib treatment differ in patients with newly diagnosed and recurrent bcr-abl positive acute lymphoblastic leukemia. Leukemia. 2012; 26(7):1475-81.
44. He C, Zheng L, Xu Y, Liu M, Li Y, Xu J. Highly sensitive and noninvasive detection of epidermal growth factor receptor T790M mutation in non-small cell lung cancer. Clinica chimica acta; international journal of clinical chemistry. 2013; 425:119-24.
45. Inukai M, Toyooka S, Ito S, Asano H, Ichihara S, Soh J, et al. Presence of epidermal growth factor receptor gene T790M mutation as a minor clone in non-small cell lung cancer. Cancer research. 2006; 66(16):7854-8.
46. Su K Y, Chen H Y, Li K C, Kuo M L, Yang J C, Chan W K, et al. Pretreatment epidermal growth factor receptor (EGFR) T790M mutation predicts shorter EGFR tyrosine kinase inhibitor response duration in patients with non-small-cell lung cancer. Journal of clinical oncology: official journal of the American Society of Clinical Oncology. 2012; 30(4):433-40.
47. Turke A B, Zejnullahu K, Wu Y L, Song Y, Dias-Santagata D, Lifshits E, et al. Preexistence and clonal selection of MET amplification in EGFR mutant NSCLC. Cancer cell. 2010; 17(1):77-88.
48. Diaz L A, Jr., Williams R T, Wu J, Kinde I, Hecht J R, Berlin J, et al. The molecular evolution of acquired resistance to targeted EGFR blockade in colorectal cancers. Nature. 2012; 486(7404):537-40.
49. Langdon S R, Westwood I M, van Montfort R L, Brown N, Blagg J. Scaffold-focused virtual screening: prospective application to the discovery of TTK inhibitors. J Chem Inf Model. 2013; 53(5):1100-12.
50. PCT Application WO2011016472 A1
51. Schmidt M, Budirahardja Y, Klompmaker R, Medema R H. Ablation of the spindle assembly checkpoint by a compound targeting Mps1. EMBO Rep. 2005, 6, 866-872.
52. Santaguida S, Tighe A, D'Alise A M, Taylor S S, Musacchio A. Dissecting the role of MPS1 in chromosome biorientation and the spindle checkpoint through the small molecule inhibitor reversine. J. Cell Biol. 2010, 190, 73-87.
53. Kusakabe K, Ide N, Daigo Y, Tachibana Y, Itoh T, Yamamoto T, et al. Indazole-based potent and cell-active Mps1 kinase Inhibitors: Rational design from pan-kinase inhibitor Anthrapyrazolone (SP600125). J. Med. Chem. 2013, 56, 4343-4356.
54. Branford S, Rudzki Z, Walsh S, Grigg A, Arthur C, Taylor K, et al. High frequency of point mutations clustered within the adenosine triphosphate-binding region of BCR/ABL in patients with chronic myeloid leukemia or Ph-positive acute lymphoblastic leukemia who develop imatinib (STI571) resistance. Blood. 2002, 99, 3472-3475.
55. Liu Y, Lang Y, Patel N K, Ng G, Laufer R, et al. The Discovery of Orally Bioavailable Tyrosine Threonine Kinase (TTK) Inhibitors: 3-(4-(heterocyclyl)phenyl)-1H-indazole-5-carboxamides as Anticancer Agents. J Med Chem. 2015, Apr. 3 [Epub ahead of print].
56. Estrada A A, Liu X, Baker-Glenn C, Beresford A, Burdick D J, et al. Discovery of highly potent, selective, and brain-penetrable leucine-rich repeat kinase 2 (LRRK2) small molecule inhibitors. J Med Chem. 2012, 55(22), 9416-9433.

1. A monopolar spindle 1 kinase (MPS1) inhibitor for use in a method of treating cancer in a human subject, wherein the method comprises:
(a) determining in a sample obtained from the subject whether a population of cells contains a MPS1 gene or a MPS1 protein that comprises one or more naturally occurring mutations as compared to the nucleic acid sequence of SEQ ID NO: 2 or the amino acid sequence of SEQ ID NO: 1, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y568C and p.C604W;
(b) selecting a MPS1 kinase inhibitor effective for use in treating the subject that is not associated with the development of acquired drug resistance that is correlated with the presence of the one or more naturally occurring mutations in the MPS1 gene or the MPS1 protein in step (a); and
(c) treating the subject with a therapy protocol that comprises administering the MPS1 kinase inhibitor selected in step (b).

2. A method of treating a human cancer subject with a therapy protocol that comprises administration of a first monopolar spindle 1 kinase (MPS1) kinase inhibitor to the subject, the method comprising:
(a) determining in a sample obtained from the subject whether a population of cells contains a MPS1 gene or a MPS1 protein that comprises one or more naturally occurring mutations, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y568C and p.C604W;
(b) selecting a MPS1 kinase inhibitor effective for use in treating the subject that is not associated with the development of acquired drug resistance that is correlated with the presence of the one or more naturally occurring mutations in the MPS1 gene or the MPS1 protein in step (a); and
(c) treating the subject with the MPS1 kinase inhibitor selected in step (b).

3. A method of selecting a monopolar spindle 1 kinase (MPS1) kinase inhibitor for use in treating cancer in a human subject, the method comprising:
(a) determining in a sample obtained from the subject whether a population of cells contains a MPS1 gene or a MPS1 protein that comprises one or more naturally occurring mutations, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y568C and p.C604W;
(b) selecting a MPS1 kinase inhibitor effective for use in treating the subject that is not associated with the development of acquired drug resistance that is correlated with the presence of the one or more naturally occurring mutations in the MPS1 gene or the MPS1 protein in step (a); and
(c) treating the subject with a therapy protocol that comprises administering the MPS1 kinase inhibitor selected in step (b).

4. The monopolar spindle 1 kinase (MPS1) inhibitor for use in a method of treating cancer of claim 1, further comprising monitoring the subject during treatment with the MPS1 inhibitor to determine whether cancer cells from the subject have developed acquired drug resistance; and optionally selecting a further MPS1 kinase inhibitor for use in treating the subject.

5. A method of determining a therapy protocol using a monopolar spindle 1 kinase (MPS1) kinase inhibitor for treating cancer in a human subject, the method comprising:
(a) determining whether the subject has acquired resistance to treatment with a first MPS1 kinase inhibitor;
(b) determining in a sample obtained from the subject whether a population of cells contains a MPS1 gene or a MPS1 protein that comprises one or more naturally occurring mutations, wherein the naturally occurring mutations in the MPS1 gene are selected from c.1593A>G, c.1831A>G, c.1799T>C, c.1703A>G and c.1812T>G and/or the naturally occurring mutations in the MPS1 protein are selected from p.I531M, p.S611G, p.M600T, p.Y568C and p.C604W and wherein the presence of one or more mutations is indicative of a resistance to the first MPS1 inhibitor;
(c) selecting a further MPS1 kinase inhibitor effective for use in treating the subject that is not associated with the development of acquired drug resistance that is correlated with the presence of the one or more naturally occurring mutations in the MPS1 gene or the MPS1 protein in step (b); and
(d) treating the subject with a revised therapy protocol that comprises administering the further MPS1 kinase inhibitor selected in step (c).

6. The monopolar spindle 1 kinase (MPS1) inhibitor for use in a method of treating cancer of claim 1, wherein the MPS1 kinase inhibitor is selected from AZ3156, NMS-P715, OncoTherapy Compound II, SNG12, Mps-BAY1, Mps-BAY2a, MPS-2b, SP600125, Reversine, Mps1-IN-2GNE-7915, N—((R)-Cyclopropyl(pyridin-2-yl)methyl)-3-(4-((endo)-3-hydroxy-8-azabicyclo[3.2.1]octan-8-yl)phenyl)-1H-indazole-5-carboxamide (Compound 75), Tert-Butyl 6-(2-Chloro-4-(1-methyl-1H-imidazol-5-yl) phenylamino)-2-(1-methyl-1H-pyrazol-4-yl)-1H-pyrrolo[3,2-c]pyridine-1-carboxylate (CCT251455), Isopropyl 6-(4-(1,2-dimethyl-1H-imidazol-5-yl)-2-phenylamino)-2-(1-methyl-1H-pyrazol-4-yl)-1H-pyrrolo[3,2-c]pyridine-1-carboxylate (Compound 2), or Isopropyl 6-(4-(1,2-dimethyl-1H-imidazol-5-yl)-2-fluorophenylamino)-2-(1-methyl-1H-pyrazol-4-yl)-1H-pyrrolo[3,2-c]pyridine-1-carboxylate (Compound 3).

7. The monopolar spindle 1 kinase (MPS1) inhibitor for use in a method of treating cancer of claim 1, wherein:
the presence of a c.1593A>G or p.I531M mutation correlates with the acquisition of drug resistance to a MPS1 kinase inhibitor which is an 8-oxapurine such as AZ3146, a diaminopyridine such as ONCOII, a pyrazoloquinazoline such as NMS-P715, a 1H-pyrrolo[3,2-c]pyridine compound such as CCT251455, compound 2 or compound 3; or
the presence of the c.1593A>G or p.I531M mutation selects a MPS1 kinase inhibitor which is a triaminopyridine such as SNG12 for use in treating the subject.

8. The monopolar spindle 1 kinase (MPS1) inhibitor for use in a method of treating cancer of claim 1, wherein:
the presence of c.1831A>G or p.S611G mutation correlates with the acquisition of drug resistance to a MPS1 kinase inhibitor which is an 8-oxapurine such as AZ3146, a diaminopyridine such as ONCOII, a triaminopyridine such as SNG12 and a 1H-pyrrolo[3,2-c]pyridine compound such as CCT251455; or
the presence of the c.1831A>G or p.S611G mutation selects a MPS1 kinase inhibitor which is a pyrazoloquinazoline such as NMS-P715 for use in treating the subject.

9. The monopolar spindle 1 kinase (MPS1) inhibitor for use in a method of treating cancer of claim 1, wherein:
the presence of c.1703A>G or p.Y568C mutation correlates with the acquisition of drug resistance to a MPS1 kinase inhibitor which is an 8-oxapurine such as AZ3146, a diaminopyridine such as ONCOII, a triaminopyridine such as SNG12 and a MPS1 kinase inhibitor which is a pyrazoloquinazoline such as NMS-P715; or
the presence of the c.1703A>G or p.Y568C mutation selects a 1H-pyrrolo[3,2-c]pyridine compound such as CCT251455 for use in treating the subject.

10. The monopolar spindle 1 kinase (MPS1) inhibitor for use in a method of treating cancer of claim 1, wherein:
the presence of c.1812T>G or p.C604W mutation correlates with the acquisition of drug resistance to a MPS1 kinase inhibitor which is an 8-oxapurine such as AZ3146, a diaminopyridine such as ONCOII, a triaminopyridine such as SNG12 and a MPS1 kinase; or inhibitor which is a pyrazoloquinazoline such as NMS-P715; or
the presence of the c.1812T>G or p.C604W mutation selects a 1H-pyrrolo[3,2-c]pyridine compound such as Compound 2 or Compound 3 for use in treating the subject.

11. The monopolar spindle 1 kinase (MPS1) inhibitor for use in a method of treating cancer of claim 1, wherein the cancer is breast cancer, pancreatic cancer, ovarian cancer, lung cancer, colon cancer, bladder cancer, thyroid cancer, pancreatic ductal adenocarcinoma, glioblastoma and a haematological cancer.

12. The monopolar spindle 1 kinase (MPS1) inhibitor for use in a method of treating cancer as claimed in claim 1, wherein the MPS1 kinase has at least 90% amino acid sequence identity with SEQ ID NO: 1 or the MPS1 gene has at least 90% nucleotide sequence identity with SEQ ID NO: 2.

13. The monopolar spindle 1 kinase (MPS1) inhibitor for use in a method of treating cancer as claimed in claim 1, wherein the MPS1 kinase comprises the amino acid sequence of SEQ ID NO: 1 or is encoded by the nucleic acid sequence of SEQ ID NO: 2.

14. The monopolar spindle 1 kinase (MPS1) inhibitor for use in a method of treating cancer as claimed in claim 1, wherein the sample is a cancer cell sample and the method further comprises the step of processing the cancer cell sample to produce a DNA sample or a protein sample.

15. The monopolar spindle 1 kinase (MPS1) inhibitor for use in a method of treating cancer as claimed in claim 1, wherein the sample is a DNA sample or a protein sample.

16. The monopolar spindle 1 kinase (MPS1) inhibitor for use in a method of treating cancer as claimed in claim 1, wherein determining the presence of the naturally occurring mutations in the MPS1 gene comprises using one or more of PCR/sequencing, or single nucleotide polymorphism assays such as droplet digital PCR (ddPCR).

17. The monopolar spindle 1 kinase (MPS1) inhibitor for use in a method of treating cancer as claimed in claim 1, wherein the method comprises the initial step of obtaining a sample from said individual.

18. The monopolar spindle 1 kinase (MPS1) inhibitor for use in a method of treating cancer as claimed in claim 1, wherein treatment with the MPS1 kinase inhibitor is combined with treatment with a further anti-cancer therapy.

19. The monopolar spindle 1 kinase (MPS1) inhibitor for use in a method of treating cancer as claimed in claim 1, wherein treatment with MPS1 kinase inhibitor is used in conjunction with a further chemotherapeutic agent.

20. The monopolar spindle 1 kinase (MPS1) inhibitor for use in a method of treating cancer as claimed in claim 1, wherein the further chemotherapeutic agent is Amsacrine (Amsidine), Bleomycin, Busulfan, Capecitabine (Xeloda), Carboplatin, Carmustine (BCNU), Chlorambucil(Leukeran), Cisplatin, Cladribine(Leustat), Clofarabine (Evoltra), Crisantaspase (Erwinase), Cyclophosphamide, Cytarabine (ARA-C), Dacarbazine (DTIC), Dactinomycin (Actinomycin D),Daunorubicin, Docetaxel (Taxotere), Doxorubicin, Epirubicin, Etoposide (Vepesid, VP-16), Fludarabine (Fludara), Fluorouracil (5-FU), Gemcitabine (Gemzar), Hydroxyurea (Hydroxycarbamide, Hydrea),Idarubicin (Zavedos). Ifosfamide (Mitoxana), Irinotecan (CPT-11, Campto), Leucovorin (folinic acid), Liposomal doxorubicin (Caelyx, Myocet), Liposomal daunorubicin (DaunoXome®) Lomustine, Melphalan, Mercaptopurine, Mesna, Methotrexate, Mitomycin, Mitoxantrone, Oxaliplatin (Eloxatin), Paclitaxel (Taxol), Pemetrexed (Alimta), Pentostatin (Nipent), Procarbazine, Raltitrexed (Tomudex®), Streptozocin (Zanosar®), Tegafur-uracil (Uftoral), Temozolomide (Temodal), Teniposide (Vumon), Thiotepa, Tioguanine (6-TG) (Lanvis), Topotecan (Hycamtin), Treosulfan, Vinblastine (Velbe), Vincristine (Oncovin), Vindesine (Eldisine) or Vinorelbine (Navelbine).

21. The monopolar spindle 1 kinase (MPS1) inhibitor for use in a method of treating cancer as claimed in 2, further comprising monitoring the subject during treatment with the MPS1 inhibitor to determine whether cancer cells from the subject have developed acquired drug resistance; and optionally selecting a further MPS1 kinase inhibitor for use in treating the subject.

22. The monopolar spindle 1 kinase (MPS1) inhibitor for use in a method of treating cancer as claimed in claim 3, further comprising monitoring the subject during treatment with the MPS1 inhibitor to determine whether cancer cells from the subject have developed acquired drug resistance; and optionally selecting a further MPS1 kinase inhibitor for use in treating the subject.
